(19) United States
(12) Patent Application Publication
(10) Pub. No.: US 2020/0276211 A1
(43) Pub. Date: Sep. 3, 2020

(54) COMPOUNDS FOR TREATING NEAR VISION DISORDERS

(71) Applicant: VIEWPOINT THERAPEUTICS, INC., San Francisco, CA (US)

(72) Inventor: Paul Ross FATHEREE, San Francisco, CA (US)

(21) Appl. No.: 16/762,638

(22) PCT Filed: May 28, 2018
(86) PCT No.: PCT/US2018/062877

§ 371 Date: May 8, 2020

Related U.S. Application Data
(60) Provisional application No. 62/591,717, filed on Nov. 28, 2017.

(51) Int. Cl. A61K 31/58; C07J 41/00; C07J 43/00; C07J 9/00; A61K 31/575; A61K 31/385; A61K 9/00; A61P 27/10

(52) U.S. Cl. CPC A61K 31/58; C07J 41/0088; C07J 43/003; C07J 9/00; A61P 27/10; A61K 31/575; A61K 31/385; A61K 9/0048; C07J 41/0055

(57) ABSTRACT

Cataract and presbyopia affect billions of people worldwide. Described herein are new compounds and methods of treating and preventing these diseases.

### CROSS-REFERENCE TO RELATED APPLICATIONS

This application claims the priority of U.S. Provisional Patent Application No. 62/591,717, filed Nov. 28, 2017, the entire contents of which are incorporated herein by reference.

### BACKGROUND OF THE INVENTION

Cataract and presbyopia are types of vision disorders. Cataract is the clouding of the lens in the eye that affects vision. Presbyopia is age-related far-sightedness that generally manifests between the ages of 40 and 50. Presbyopia initially causes blurred vision, difficulty seeing in dim light, and eye strain. In healthy eyes, the lens is able to focus light from objects at different distances by a process called accommodation. During accommodation, muscles surrounding the lens contract, causing the lens to change shape and increasing the focusing power of the eye. With increasing age, the lens becomes stiffer as its structural crystallin proteins become misfolded. The increased lens stiffness limits the eye's ability to focus for reading or other tasks that require clear vision at near distances.

Unfortunately, cataract affects more than 24 million Americans aged 40 years and older and presbyopia affects approximately 112 million Americans. The conventional treatment for cataract is surgical replacement of the lens with an artificial lens. Surgical treatment of cataract is costly and artificial lenses do not have the same optical qualities as a normal lens. Surgical options are also available to treat presbyopia. New treatment options are needed to address cataract and presbyopia.

### SUMMARY OF THE INVENTION

Cataract and presbyopia affect billions of people worldwide. Hence, there is a need for new methods of treating and preventing these diseases. The disclosure provides new compounds, salts, compositions and uses thereof in the treatment of near vision disorders.

In certain aspects, the disclosure provides a compound of Formula (I):

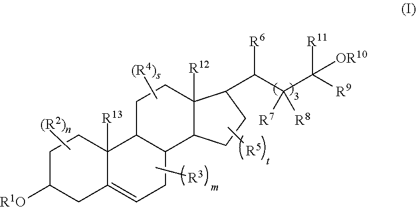

or a salt thereof, wherein:

R¹ and R¹⁰ are independently selected from hydrogen, —C(O)(CR²⁰₂)₁₋₆NR²¹₂, —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)₂, wherein at least one of R¹ and R¹⁰ is not hydrogen;

R², R³, R⁴, and R⁵ are independently selected at each occurrence from halogen, —OR¹⁵, —SR¹⁵, —OSO₃R¹⁵, —OPO₃R¹⁵, —N(R¹⁵)₂, —C(O)R¹⁵, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN;

R⁷ and R⁸ are independently selected at each occurrence from hydrogen, halogen, —OR¹⁵, —SR¹⁵, —OSO₃R¹⁵, —OPO₃R¹⁵, —N(R¹⁵)₂, —C(O)R¹⁵, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN;

n, s, m, and t are independently selected from 0, 1, 2, 3, 4, or 5;

R⁶, R⁹, R¹¹, R¹², and R¹³ are independently selected at each occurrence from C₁-C₆ alkyl optionally substituted with one or more substituents independently selected from halogen, —OR¹⁵, —SR¹⁵, —N(R¹⁵)₂, —NO₂, and —CN;

R¹⁵ is independently selected at each occurrence from hydrogen, and C₁-C₆ alkyl optionally substituted with one or more substituents independently selected from halogen, —CN, —NO₂, —NH₂, —OH, and —OCH₃;

R²⁰ is independently selected at each occurrence from hydrogen, halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —SR²², —S—S—R²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —S—C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

or two R²¹ groups are taken together with the atoms to which they are attached form a heterocycle, optionally substituted with one or more R²⁴;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²³ is selected from:
—C₁₋₆alkylene-(X¹—C₁₋₆alkylene)₁₋₂₄-X¹—C₁₋₆alkyl, wherein X¹ is independently selected at each occurrence from O, S, and NH and wherein R²³ is optionally substituted on a carbon or nitrogen atom with one or more substituents independently selected from R²⁴; and a 3- to 12-membered heterocycle optionally substituted with one or more substituents independently selected from R²⁴;

R²⁴ is independently selected at each occurrence from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and

X² is independently selected at each occurrence from O and NH.

The disclosure provides pharmaceutical compositions of compounds or salts of Formula (I) and a pharmaceutically acceptable carrier. In certain embodiments, the pharmaceutical composition is an eye drop.

The disclosure provides methods of treating or preventing a near vision disorder in a subject in need thereof, comprising administering to the subject a pharmaceutical composition described herein. The near-vision disorder may be selected from cataract and presbyopia. The cataract may be selected from nuclear cataract, cortical cataract, posterior capsular cataract, congenital cataract, early-onset hereditary cataract, metabolic cataract, secondary cataract, blunt traumatic cataract, penetrating traumatic cataract, post-vitrectomy cataract, and radiation-induced cataract. The presbyopia may be incipient presbyopia, functional presbyopia, absolute presbyopia, premature presbyopia and nocturnal presbyopia. In certain embodiments, administration of the pharmaceutical composition involves the topical administration of the pharmaceutical composition, e.g., to the surface of the eye of said subject. In certain embodiments, the method further comprises administering an antioxidant, e.g., alpha-lipoic acid or a prodrug thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 depicts protein thermal stability changes in cryAB with compounds 1.18, 1.21, or 1.25;

FIG. 2 depicts the fraction of compound 1.18, 1.21, or 1.25 bound to cryAB (R120G) as a function of concentration; and

FIG. 3 depicts kinetic disaggregation of Thioflavin T-positive cryAB amyloids in human lens homogenates with compound 1.18, 1.21, 1.25 or control.

### DETAILED DESCRIPTION OF THE INVENTION

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain radical consisting solely of carbon and hydrogen atoms, containing no unsaturation, and preferably having from one to ten carbon atoms (i.e., C₁-C₁₀ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to ten carbon atoms (i.e., C₅-C₁₀ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (isobutyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more substituents such as those substituents described herein.

“Alkenyl” refers to a straight or branched hydrocarbon chain radical group consisting solely of carbon and hydrogen atoms, containing at least one carbon-carbon double bond, and preferably having from two to 10 carbon atoms (i.e., C₂-C₁₀ alkenyl). In certain embodiments, an alkenyl comprises two to eight carbon atoms (i.e., C₂-C₈ alkenyl). In other embodiments, an alkenyl comprises two to six carbon atoms (i.e., C₂-C₆ alkenyl). The alkenyl may be attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to a straight or branched hydrocarbon chain radical group consisting solely of carbon and hydrogen atoms, containing at least one carbon-carbon triple bond, and preferably having from two to ten carbon atoms (i.e., C₂-C₁₀ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl may be attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents such as those substituents described herein.

“Alkylene” refers to straight or branched divalent hydrocarbon chain linking the rest of the molecule to a radical group, consisting solely of carbon and hydrogen, containing no unsaturation, and preferably having from one to six carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain, or in the case of methylene, the methylene carbon is attached through one bond to the rest of the molecule and through one bond to the radical group. In certain embodiments, an alkylene comprises one to six carbon atoms (i.e., C₁-C₆ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises four to six carbon atoms (i.e., C₄-C₆ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

The term “C_{x-y}” when used in conjunction with a chemical moiety, such as alkyl, alkenyl, or alkynyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain. The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond respectively.

“Haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally substituted as described herein.

“Carbocycle” refers to saturated, unsaturated or aromatic rings in which each atom of the ring is carbon. Carbocycle may be monocyclic or polycyclic and may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle is optionally substituted by one or more substituents such as those substituents described herein.

“Heterocycle” refers to a saturated, unsaturated or aromatic ring comprising carbon atoms and one or more heteroatoms in the ring. Exemplary heteroatoms include N, O, Si, P, B, and S atoms. Heterocycle may be monocyclic or polycyclic and may include 3- to 10-membered monocyclic rings, 6- to 12-membered bicyclic rings, and 6- to 12-membered bridged rings. For a polycyclic heterocycle, at least one ring of the polycycle includes a heteroatom in the ring. Each ring of a bicyclic heterocycle may be selected from saturated, unsaturated, and aromatic rings. In some embodiments, the heterocycle is a heteroaryl. In some embodiments, the heterocycle is a heterocycloalkyl. In an exemplary embodiment, a heterocycle, e.g., pyridyl, may be fused to a saturated or unsaturated ring, e.g., cyclohexane, cyclopentane, or cyclohexene. Unless stated otherwise specifically in the specification, a heterocycle is optionally substituted by one or more substituents such as those substituents described herein.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, ptoluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier is “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b-}N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b-}N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### INTRODUCTION

Alpha-crystallin is a maj or structural protein found in the eye and can maintain the refractive index and transparency of the lens. Alpha-crystallin is composed of two homologous subunits: alphaA-crystallin (cryAA) and alphaB-crystallin (cryAB), which belong to a family of small heat shock proteins (sHSPs) that contain a conserved crystalline domain. AlphaA-crystallin is 173 amino acids long and alphaB-crystallin is 175 amino acids long. The two alpha crystallin genes, alphaA- and alphaB-, encode for proteins that share 57% sequence identity. The ratio of alphaA-crystallin to alphaB-crystallin in most vertebrate lenses can be 3:1 but this ratio can vary with species and age. The alphaA-crystallin protein can be found mostly in the lens and only in few other tissues whereas alphaB-crystallin protein can be ubiquitously expressed and can be found in other tissues, such as brain, heart and muscle.

These alpha-crystallin subunits act as molecular chaperones to prevent the cellular aggregation and inactivation of client proteins under a variety of stress conditions. However, the chaperone activity of these alpha-crystallin subunits can be lost or deteriorated during aging or due to certain genetic or environment factors, which can cause aggregation and precipitation of alpha-crystallin and lead to cataracts.

In certain embodiments, the disclosure provides compounds, formulations and methods for treating vision disorders associated with alpha-crystallin protein aggregation in the lens. In particular, the disclosure provides compounds, formulations and methods for treating cataract and presbyopia.

### COMPOUNDS OF THE DISCLOSURE

The present disclosure provides compounds, salts, and formulations thereof, for use in the treatment of ophthalmic diseases. The disclosed compounds and salts can be used, for example, for the treatment or prevention of vision disorders such as near vision impairment. In certain embodiments, the compounds of the disclosure inhibit, reduce, or reverse alpha-crystallin protein aggregation in the lens of an eye. Compounds and salts of the disclosure may be used in the formulations, methods and combination therapies described herein. In certain embodiments, compounds and salts of the disclosure are used in the treatment or prevention of cataract or presbyopia.

In certain embodiments, the compounds and salts of the disclosure are sterols. Some sterols, such as cholesterol, display poor aqueous solubility, e.g., less than 0.5 mg/100 mL of water, making therapeutic administration challenging. In certain aspects, the disclosure provides modifications of sterols to increase aqueous solubility and improve bioavailability of sterol compounds.

In some embodiments, the compound or salt of the disclosure is a prodrug of a sterol. In certain embodiments, the aqueous solubility of the prodrug is greater than the aqueous solubility of the sterol. For example, the prodrug form of a sterol may have an aqueous solubility that is about 10% or more, 20% or more, 30% or more, 40% or more, 50% or more, from 10% to 80%, from 10% to 70%, from 10% to 60%, or from 10% to 50% greater than the aqueous solubility of the sterol.

In some embodiments, the compound or salt of the disclosure is an analog of a sterol, e.g., an analog of a sterol with an increased number of polar or hydrogen bonding groups to improve aqueous solubility. In certain embodiments, the aqueous solubility of the analog is greater than the aqueous solubility of the sterol. For example, the analog of a sterol may have an aqueous solubility that is about 10% or more, 20% or more, 30% or more, 40% or more, 50% or more, from 10% to 80%, from 10% to 70%, from 10% to 60%, or from 10% to 50% greater than the aqueous solubility of the sterol. In certain embodiments, the analog is not a prodrug.

In some embodiments, the compound or salt of the disclosure is an analog or prodrug of a sterol, wherein the cellular uptake of the analog or prodrug is greater than the cellular uptake of the sterol. For example, the analog or prodrug form of a sterol may have a cellular uptake of about 10% or more, 20% or more, 40% or more, 50% or more, from 10% to 80%, from 10% to 70%, from 10% to 60%, or from 10% to 50%, greater than the cellular uptake of the sterol.

In certain embodiments, the compound or salt of the disclosure is an analog or prodrug of a sterol, wherein the corneal diffusion of the analog or prodrug is greater than the corneal diffusion of the sterol. For example, the analog or prodrug form of a sterol may have a corneal diffusion of about 10% or more, 20% or more, 40% or more, 50% or more, from 10% to 80%, from 10% to 70%, from 10% to 60%, or from 10% to 50% greater than the corneal diffusion of the sterol.

In certain embodiments, the disclosure provides a sterol such as cholesterol, ergosterol, 25-hydroxycholesterol, lanosterol or any other naturally or non-naturally occurring sterol described herein or otherwise wherein the sterol is modified with a substituent on the C3 hydroxyl. In certain embodiments, the compound is modified with a polarity or solubility enhancing moiety such as a moiety comprising one or more hydrogen bonding groups is attached to the oxygen at the C3 position of the sterol. In certain embodiments, the hydrogen bonding group is part of an amine or a carboxylate. In certain embodiments, the sterol is modified with a moiety comprising at least one amine, e.g., a primary amine, a secondary amine, a tertiary amine or a quaternary amine, or carboxylate at the C3 hydroxyl. For example, a sterol (25-hydroxycholesterol) modified with a moiety comprising at least one amine or carboxylate includes compounds 1.18, 1.21 and 1.25 described herein. In certain embodiments, the sterol is modified with a moiety comprising at least one amine or carboxylate on a hydroxyl of the sterol other than at the C3 position, e.g., the C25 hydroxyl of 25-hydroxycholesterol. In certain embodiments, a modified sterol of the disclosure is cationic at physiological pH or includes a quaternary nitrogen. In certain embodiments, a modified sterol of the disclosure is anionic at physiological pH.

In certain embodiments, a compound of the disclosure is represented by Formula (I):

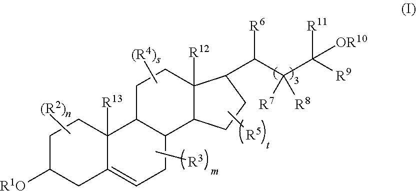

or a salt thereof, wherein:

R¹ and R¹⁰ are independently selected from hydrogen, —C(O)(CR²⁰₂)₁₋₆NR²¹₂, —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)₂, wherein at least one of R¹ and R¹⁰ is not hydrogen;

R², R³, R⁴, and R⁵ are independently selected at each occurrence from halogen, —OR¹⁵, —SR¹⁵, —OSO₃R¹⁵, —OPO₃R¹⁵, —N(R¹⁵)₂, —C(O)R¹⁵, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN;

R⁷ and R⁸ are independently selected at each occurrence from hydrogen, halogen, —OR¹⁵, —SR¹⁵, —OSO₃R¹⁵, —OPO₃R¹⁵, —N(R¹⁵)₂, —C(O)R¹⁵, —C(O)OR¹⁵, —C(O)N(R⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN;

n, s, m, and t are independently selected from 0, 1, 2, 3, 4, or 5;

R⁶, R⁹, R¹¹, R¹², and R¹³ are independently selected from C₁-C₆ alkyl optionally substituted with one or more substituents independently selected from halogen, —OR¹⁵, —SR¹⁵, —N(R¹⁵)₂, —NO₂, and —CN;

R¹⁵ is independently selected at each occurrence from hydrogen, and C₁-C₆ alkyl optionally substituted with one or more substituents independently selected from halogen, —CN, —NO₂, —NH₂, —OH, and —OCH₃;

R²⁰ is independently selected at each occurrence from hydrogen, halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²⁰ is independently optionally substituted with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —SR²², —S—S—R²², —N(R²²)₂, —N(R²²)₃⁺, —NR²²C(═N)N(R²²)₂, —S(═O)R²², —S(═O)₂R²², —S(═)₂N(R²²)₂, —NR²²S(═O)₂R²², —S—C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²¹ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

or two R²¹ groups are taken together with the atoms to which they are attached form a heterocycle, optionally substituted with one or more R²⁴;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²³ is selected from:

—C₁₋₆alkylene-(X¹—C₁₋₆alkylene)₁₋₂₄-X¹—C₁₋₆alkyl, wherein X¹ is independently selected at each occurrence from O, S, and NH and wherein R²³ is optionally substituted on a carbon or nitrogen atom with one or more substituents independently selected from R²⁴; and
a 3- to 12-membered heterocycle optionally substituted with one or more substituents independently selected from R²⁴;

R²⁴ is independently selected at each occurrence from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²⁴ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and

X² is independently selected at each occurrence from O and NH.

In certain embodiments, a compound of Formula (I) is represented by any of the following Formulas (IIA), (IIB), (IIC), (IID), (IIE), (IIF), (IIG), (IIH), (IIJ), (IIK) and (IIL):

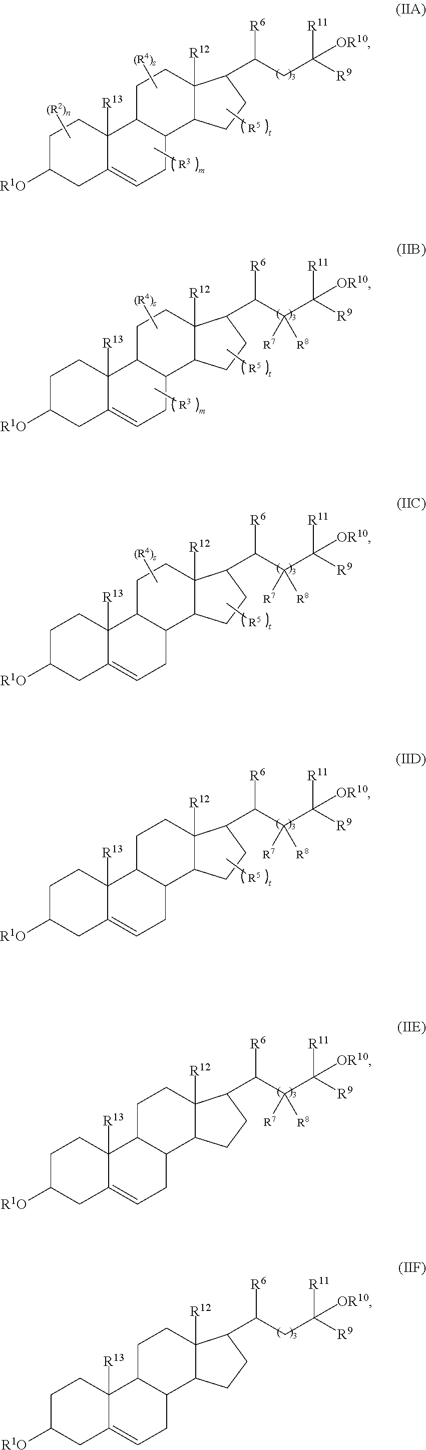

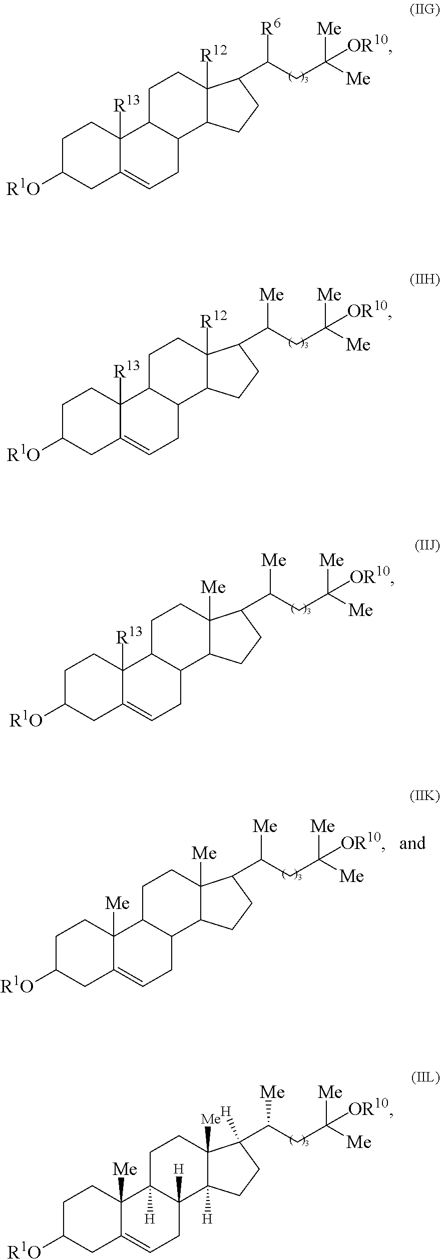

or a salt of any one thereof.

For a compound or salt of Formula (I), (IIA), (IIB), (IIC), (IID), (IIE), (IIF), (IIG), (IIH), (IIJ), (IIK), (IIL), (III), (IV) and (V), R¹ may be selected from —C(O)(CR²⁰₂)₁₋₆NR²¹₂, —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)₂ and R¹⁰ is H.

For a compound or salt of Formula (I), (IIA), (IIB), (IIC), (IID), (IIE), (IIF), (IIG), (IIH), (IIJ), (IIK), (IIL), and (III), R¹ and R¹⁰ may be independently selected from H and —C(O)(CR²⁰₂)₁₋₆NR²¹₂. For a compound or salt of any one of Formulas (I), (IIA), (IIB), (IIC), (IID), (IIE), (IIF), (IIG), (IIH), (IIJ), (IIK), (IIL), and (III), R¹ may be selected from —C(O)(CR²⁰₂)₁₋₆NR²¹₂ and R¹⁰ is H In certain embodiments, for a compound or salt of any one of Formulas (I), (IIA), (IIB), (IIC), (IID), (IIE), (IIF), (IIG), (IIH), (IIJ), (IIK), (IIL), and (III), R¹ is —C(O)(CR²⁰₂)₁₋₆NR²¹₂ which may be further selected from —C(O)CR²⁰₂NR²¹₂, —C(O)(CR²⁰₂)₂NR²¹₂, —C(O)(CR²⁰₂)₃NR²¹₂, —C(O)(CR²⁰₂)₄NR²¹₂, —C(O)(CR²⁰₂)₅NR²¹₂, and —C(O)(CR²⁰₂)₆NR²¹₂ and R¹⁰ is H.

In certain embodiments for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)CR²⁰₂NR²¹₂. In certain embodiments, R¹ and R¹⁰ are independently selected from H and —C(O)(CR²⁰₂)₂NR²¹₂. In certain embodiments, R¹ and R¹⁰ are independently selected from H and —C(O)(CR²⁰₂)₃NR²¹₂. In certain embodiments, R¹ and R¹⁰ are independently selected from H and —C(O)(CR²⁰₂)₄NR²¹₂. In certain embodiments, R¹ and R¹⁰ are independently selected from H and —C(O)(CR²⁰₂)₅NR²¹₂. In certain embodiments, R¹ and R¹⁰ are independently selected from H and —C(O)(CR²⁰₂)₆NR²¹₂.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)CR²⁰₂NR²¹₂, wherein —C(O)CR²⁰₂NR²¹₂ may be further selected from:

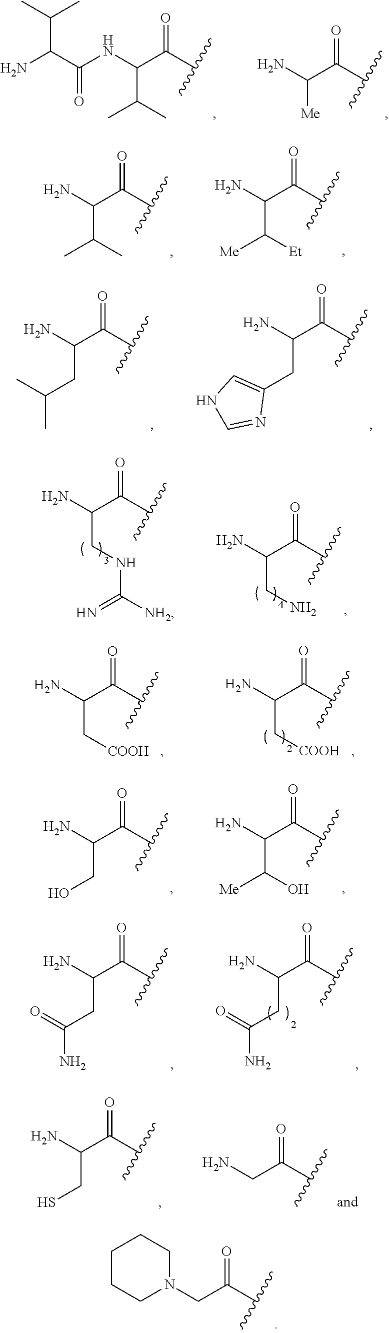

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)CR²⁰₂NR²¹₂, wherein —C(O)CR²⁰₂NR²¹₂ may be further selected from:

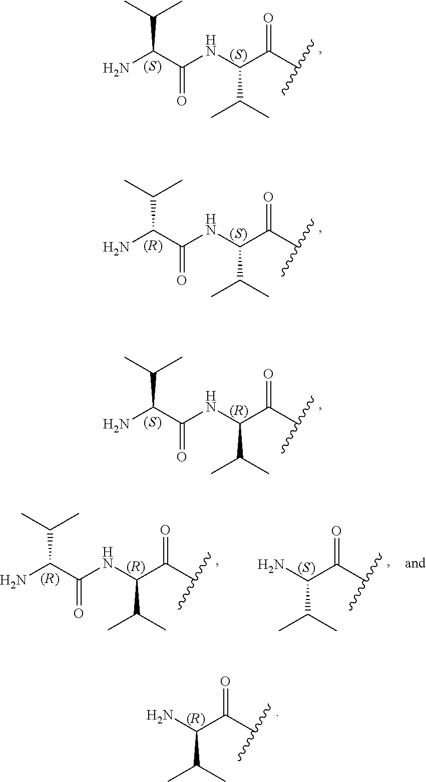

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)(CR²⁰₂)₁₋₆NR²¹₂, wherein —C(O)(CR²⁰₂)₁₋₆NR²¹₂ may be selected from:

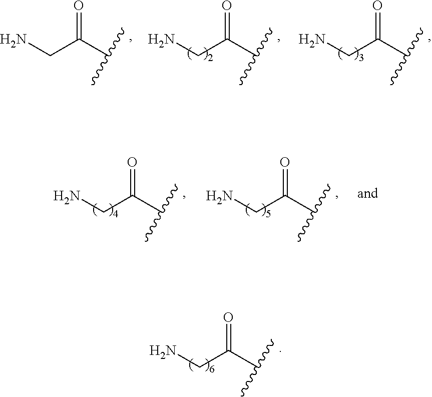

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and

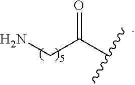

In certain embodiments, for a compound or salt of any one of Formulas (I), (IIA), (IIB), (IIC), (IID), (IIE), (IIF) (IIG), (IIH), (IIJ), (IIK), (IIL), and (III), R¹ and R¹⁰ are independently selected from H and —C(O)R²³. In certain embodiments, for a compound or salt of Formula (I), R¹ and R¹⁰ are independently selected from H and —C(O)R²³. In certain embodiments, each R²³ is independently selected from —C₁₋₆alkylene-(X¹—C₁₋₆alkylene)₁₋₂₄-X¹—C₁₋₆alkyl. In certain embodiments, each R²³ is independently selected from —C₁₋₆alkylene-(X¹—C₁₋₆ alkylene)₁₋₂₄-X¹ C₁₋₆alkyl, —C₂alkylene-(X¹—C₁₋₆alkylene)₁₋₂₄-X¹—C₁₋₆alkyl, —C₃alkylene-(X¹—C₁₋₆ alkylene)₁₋₂₄-X¹—C₁₋₆alkyl, —C₄alkylene-(X¹—C₁₋₆alkylene)₁₋₂₄-X¹—C₁₋₆alkyl, —C₁₋₆alkylene-(X¹—C₁₋₆ alkylene)₁₋₂₄-X¹—C₁₋₆alkyl, and —C₆alkylene-(X¹—C₁₋₆alkylene)₁₋₂₄-X¹—C₁₋₆alkyl. In certain embodiments, each R²³ is independently selected from —C₁₋₆alkylene-(X¹—C₁₋₆alkylene)₁₋₂₄-X—C₁₋₆alkyl. In certain embodiments, each R²³ is independently selected from —C₁₋₆alkylene-(X—C₁₋₆alkylene)₂-X¹—C₁₋₆alkyl, —C₁₋₆alkylene-(X¹—C₁₋₆alkylene)₅-X¹—C₁₋₆alkyl, —C₁₋₆alkylene-(X¹—C₁₋₆ alkylene)₁₁-X¹—C₁₋₆alkyl, and —C₁₋₆alkylene-(X¹—C₁₋₆alkylene)₁₆-X¹—C₁₋₆alkyl.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)R²³ and each R²³ is independently selected from —C₂alkylene-(X—C₁₋₆alkylene)₁₋₂₄-X¹—C₁₋₆alkyl. In certain embodiments, each R²³ is independently selected from —C₂alkylene-(X¹—C₁₋₆alkylene)₂-X¹—C₁₋₆alkyl, —C₂alkylene-(X¹—C₁₋₆alkylene)₅-X¹—C₁₋₆alkyl, —C₂alkylene-(X¹—C₁₋₆alkylene)₁₆-X¹—C₁₋₆alkyl, and —C₂alkylene-(X¹—C₁₋₆alkylene)₁₆-X¹—C₁₋₆alkyl.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)R²³ and each R²³ is independently selected from —C₃alkylene-(X—C₁₋₆alkylene)₁₋₂₄-X¹—C₁₋₆alkyl. In certain embodiments, each R²³ is independently selected from —C₃alkylene-(X—C₁₋₆alkylene)₂-X¹—C₁₋₆alkyl, —C₃alkylene-(X—C₁₋₆alkylene)₅-X¹—C₁₋₆alkyl, —C₃alkylene-(X¹—C₁₋₆alkylene)₁₁-X¹—C₁₋₆alkyl, and —C₃alkylene-(X—C₁₋₆alkylene)₁₆-X¹—C₁₋₆alkyl.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)R²³ and each R²³ is independently selected from —C₄alkylene-(X—C₁₋₆alkylene)₁₋₂₄-X¹—C₁₋₆alkyl. In certain embodiments, R²³ is selected from —C₄alkylene-(X—C₁₋₆alkylene)₂-X¹—C₁₋₆alkyl, —C₄alkylene-(X—C₁₋₆alkylene)₅-X¹—C₁₋₆alkyl, —C₄alkylene-(X—C₁₋₆alkylene)-X¹—C₁₋₆alkyl, and —C₄alkylene-(X—C₁₋₆alkylene)₁₆-X¹—C₁₋₆alkyl.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)R²³ and each R²³ is independently selected from —C₅alkylene-(X¹—C₁₋₆alkylene)₁₋₂₄-X¹—C₁₋₆alkyl. In certain embodiments, each R²³ is independently selected from —C₅alkylene-(X¹—C₁₋₆alkylene)₂-X¹—C₁₋₆alkyl, —C₅alkylene-(X¹—C₁₋₆alkylene)₅-X¹—C₁₋₆alkyl, —C₅alkylene-(X¹—C₁₋₆alkylene)₁₁-X¹—C₁₋₆alkyl, and —C₅alkylene-(X¹—C₁₋₆alkylene)₁₆-X¹—C₁₋₆alkyl.

In certain embodiments, for a compound or salt of any one of Formulas (I), (IIA), (IIB), (IIC), (IID), (IIE), (IF), (IIG), (IIH), (IIJ), (IIK), (IIL), and (III), R¹ and R¹⁰ are independently selected from H and —C(O)R²³ and each R²³ is independently selected from from —C₆alkylene-(X¹—C₁₋₆alkylene)₁₋₂₄-X¹—C₁₋₆alkyl. In certain embodiments, each R²³ is independently selected from —C₆alkylene-(X¹—C₁₋₆alkylene)₂-X¹—C₁₋₆alkyl, —C₆alkylene-(X¹—C₁₋₆alkylene)₅-X¹—C₁₋₆alkyl, —C₆alkylene-(X¹—C₁₋₆alkylene)₁₁-X¹—C₁₋₆alkyl, and —C₆alkylene-(X¹—C₁₋₆alkylene)₁₆-X¹—C₁₋₆alkyl.

In certain embodiments, for a compound or salt of any one of Formulas (I), (IIA), (IIB), (IIC), (IID), (IIE), (IF), (IIG), (IIH), (IIJ), (IIK), (IIL), and (III), R¹ and R¹⁰ are independently selected from H and —C(O)R²³ wherein X¹ at each occurrence in R²³ is 0, wherein X¹ at each occurrence in R²³ is S, or wherein X¹ at each occurrence in R²³ is NH.

In certain embodiments, for a compound or salt of any one of Formulas (I), (IIA), (IIB), (IIC), (IID), (IIE), (IT), (IIG), (IIH), (IIJ), (IIK), (IIL), and (III), R¹ and R¹⁰ are independently selected from H and —C(O)R²³ and each R²³ is independently selected from a 3- to 12-membered heterocycle optionally substituted with one or more substituents independently selected at each occurrence from R²⁴. Each R²³ may be independently selected from a 3-membered heterocycle optionally substituted with one or more substituents independently selected at each occurrence from R²⁴. Each R²³ may be independently selected from a 4-membered heterocycle optionally substituted with one or more substituents independently selected at each occurrence from R²⁴. In certain embodiments, R²³ is selected from a 5-membered heterocycle optionally substituted with one or more substituents independently selected at each occurrence from R²⁴. In certain embodiments, R²³ is selected from a 6-membered heterocycle optionally substituted with one or more substituents independently selected at each occurrence from R²⁴. In certain embodiments, R²³ is selected from a 7-membered heterocycle optionally substituted with one or more substituents independently selected at each occurrence from R²⁴.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)R²³ and each R²³ is selected from aziridinyl, 2H-azirinyl, oxiranyl and thiiranyl, wherein R²³ is optionally substituted with one or more substituents independently selected at each occurrence from R²⁴. In certain embodiments, R²³ is selected from azetidinyl, 2,3-dihydroazetyl, azetyl, 1,3-diazetidinyl, oxetanyl, 2H-oxetyl, thietanyl, and 2H-thietyl, wherein R²³ is optionally substituted with one or more substituents independently selected at each occurrence from R²⁴.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)R²³ and each R²³ is independently selected from pyrrolidinyl, 3-pyrrolinyl, 2-pyrrolinyl, 2H-pyrrolyl, 1H-pyrrolyl, pyrazolidinyl, imidazolidinyl, 2-pyrazolinyl, 2-imidazolinyl, pyrazolyl, imidazolyl, 1,2,4-triazolyl, 1,2,3-triazolyl, tetrazolyl, tetrahydrofuranyl, furanyl, 1,3-dioxolanyl, tetrahydrothiophenyl, thiophenyl, oxazolyl, isoxazolyl, isothiazolyl, thiazolyl, 1,2-oxathiolanyl, 1,3-oxathiolanyl, 1,2,5-oxadiazolyl, 1,2,3-oxadiazolyl, 1,3,4-thiadiazolyl, 1,2,5-thiadiazolyl, sulfolanyl, 2,4-thiazolidinedionyl, succinimidyl, 2-oxazolidonyl, and hydantoinyl, and wherein R²³ is optionally substituted with one or more substituents independently selected at each occurrence from R²⁴. In certain embodiments, R²³ is selected from:

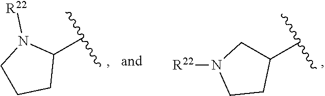

wherein R²³ is optionally substituted with one or more substituents independently selected at each occurrence from R²⁴.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)R²³ and each R²³ is independently selected from piperidinyl, pyridinyl, piperazinyl, pyridazinyl, pyrimidinyl, pyrazinyl, 1,2,4-triazinyl, 1,3,5-triazinyl, tetrahydropyranyl, 2H-pyranyl, 4H-pyranyl, pyryliumyl, 1,4-dioxanyl, 1,4-dioxinyl, thianyl, 2H-thiopyranyl, 4H-thiopyranyl, 1,3-dithianyl, 1,4-dithianyl, 1,3,5-trithianyl, morpholinyl, 4H-1,3-oxazinyl, 2H-1,2-oxazinyl, 6H-1,2-oxazinyl, 4H-1,3-oxazinyl, 2H-1,3-oxazinyl, 6H-1,3-oxazinyl, 4H-1,4-oxazinyl, 2H-1,4-oxazinyl, thiomorpholinyl, 4H-1,4-thiazinyl, 2H-1,2-thiazinyl, 6H-1,2-thiazinyl, and 2H-1,4-thiazinyl, and wherein R²³ is optionally substituted with one or more substituents independently selected at each occurrence from R²⁴. In certain embodiments, R²³ is selected from:

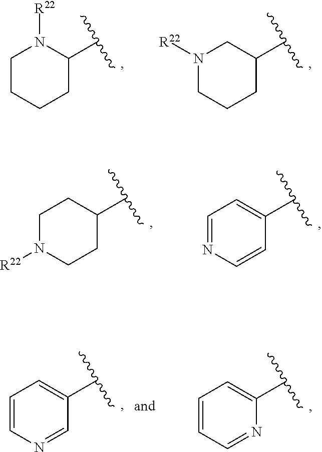

each of which may be optionally substituted with one or more substituents independently selected at each occurrence from R²⁴.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)R²³ and each R²³ is independently selected from 2,3-dihydroazepinyl, 2,5-dihydroazepinyl, 4,5-dihydroazepinyl, azepinyl, 2H-azepinyl, 3H-azepinyl, 4H-azepinyl, 1,2-diazepinyl, 1,3-diazepinyl, 1,4-diazepinyl, oxepanyl, thiepinyl, and 1,4-thiazepinyl, wherein R²³ is optionally substituted with one or more substituents independently selected at each occurrence from R²⁴.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)(C(R²⁰)₂)₁₋₆OC(O)R²⁰. In certain embodiments, R¹ and R¹⁰ are independently selected from H, —C(O)C(R²⁰)₂OC(O)R²⁰, —C(O)(C(R²⁰)₂)₂OC(O)R²⁰, —C(O)(C(R²⁰)₂)₃OC(O)R²⁰, —C(O)(C(R²⁰)₂)₄OC(O)R²⁰, —C(O)(C(R²⁰)₂)₅OC(O)R²⁰, and —C(O)(C(R²⁰)₂)₆OC(O)R²⁰.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)(C(R²⁰)₂)₁₋₆C(O)OR²¹. In certain embodiments, R¹ and R¹⁰ are independently selected from H, —C(O)C(R²⁰)₂C(O)OR²¹, —C(O)(C(R²⁰)₂)₂C(O)OR²¹, —C(O)(C(R²⁰)₂)₃C(O)OR²¹, —C(O)(C(R²⁰)₂)₄C(O)OR²¹, —C(O)(C(R²⁰)₂)₅C(O)OR²¹, and —C(O)(C(R²⁰)₂)₆C(O)OR²¹. In certain embodiments, R¹ and R¹⁰ are independently selected from H, —C(O)C(R²⁰)₂C(O)OR²¹, —C(O)(C(R²⁰)₂)₂C(O)OR²¹, —C(O)(C(R²⁰)₂)₃C(O)OR²¹, —C(O)(C(R²⁰)₂)₄C(O)OR²¹, and —C(O)(C(R²⁰)₂)₅C(O)OR²¹. In certain embodiments, R¹ and R¹⁰ are independently selected from H, —C(O)C(R²⁰)₂C(O)OR²¹, —C(O)(C(R²⁰)₂)₂C(O)OR²¹, —C(O)(C(R²⁰)₂)₃C(O)OR²¹, and —C(O)(C(R²⁰)₂)₄C(O)OR²¹. In certain embodiments, R¹ and R¹⁰ are independently selected from H, —C(O)C(R²⁰)₂C(O)OR²¹, —C(O)(C(R²⁰)₂)₂C(O)OR²¹, and —C(O)(C(R²⁰)₂)₃C(O)OR²¹. In certain embodiments, R¹ and R¹⁰ are independently selected from H, —C(O)(C(R²⁰)₂)₂C(O)OR²¹ and —C(O)(C(R²⁰)₂)₃C(O)OR²¹. In certain embodiments, for any of the preceding embodiments, one of R¹ and R¹⁰ is hydrogen.

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from: H,

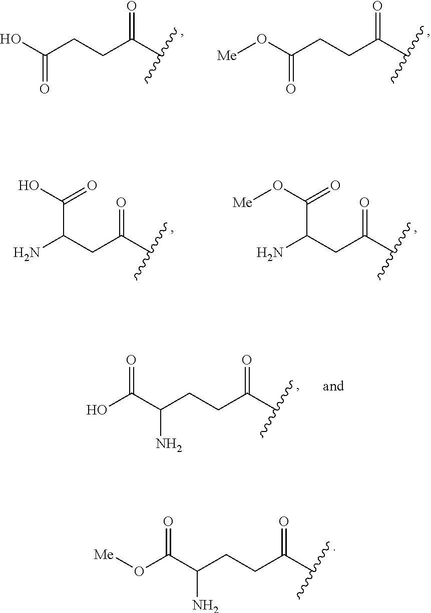 [SMILES: *C(=O)CCC(=O)O ; *C(=O)CCC(=O)OC ; *C(=O)CC(N)C(=O)O ; *C(=O)CC(N)C(=O)OC ; *C(=O)CCC(N)C(=O)O ; *C(=O)CCC(N)C(=O)OC]

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —C(O)OC(R²⁰)₃ and each R²⁰ is independently selected at each occurrence from: H,

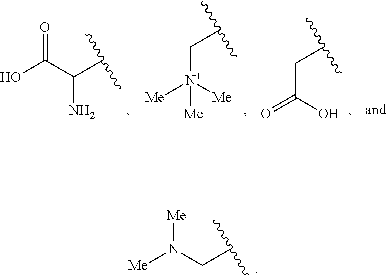 [SMILES: *C(N)C(=O)O ; *C[N+](C)(C)C ; *CC(=O)O ; *CN(C)C]

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —P(═O)(X²R²¹)₂ wherein each X² is O, or wherein each X² is NH. In certain embodiments, each R²¹ is independently selected from hydrogen and C₁₋₆ alkyl. In certain embodiments, R¹ or R¹⁰ is selected from:

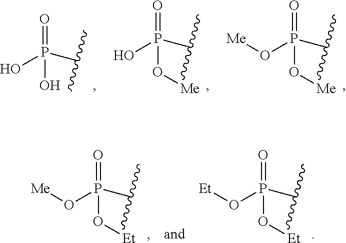 [SMILES: *P(=O)(O)O ; *P(=O)(O)OC ; *P(=O)(OC)OC ; *P(=O)(OC)OCC ; *P(=O)(OCC)OCC]

In certain embodiments, for a compound or salt of any one of Formulas (I), (IIA), (IIB), (IIC), (IID), (IIE), (IIF), (IIG), (IIH), (IIJ), (IIK), (IIL), and (III), R¹ and R¹⁰ are independently selected from H and —CH₂OP(═O)(X²R²¹)₂ wherein each X² is O or each X² is NH. In certain embodiments, each R²¹ is independently selected from hydrogen and C₁₋₆ alkyl. In certain embodiments, R¹ and R¹⁰ are independently selected from: H,

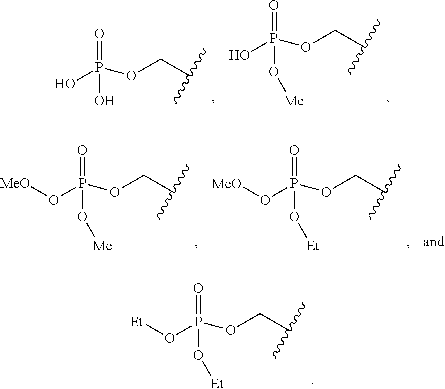 [SMILES: *COP(=O)(O)O ; *COP(=O)(O)OC ; *COP(=O)(OC)OOC ; *COP(=O)(OCC)OOC ; *COP(=O)(OCC)OCC]

In certain embodiments, for a compound or salt of Formula (I) or (IIA), R², R³, R⁴, and R⁵ are independently selected at each occurrence from halogen, —OR¹⁵, —N(R¹⁵)₂, —C(O)R¹⁵, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN. In certain embodiments, R², R³, R⁴, and R⁵ are independently selected at each occurrence from halogen, —OR¹⁵, —N(R¹⁵)₂, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN. In certain embodiments, R², R³, R⁴, and R⁵ are independently selected at each occurrence from halogen, —OR¹⁵, —N(R¹⁵)₂, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN.

In certain embodiments, for a compound or salt of Formula (I) or (IIA), R², R³, R⁴, and R⁵ are independently selected at each occurrence from halogen, —OR¹⁵, —N(R¹⁵)₂, —NO₂, —CN, and C₁-C₆ alkyl optionally substituted with one or more substituents independently selected from halogen, —OR¹⁵, —N(R¹⁵)₂, —NO₂, and —CN. In certain embodiments, R², R³, R⁴, and R⁵ are independently selected at each occurrence from —OR¹⁵, —N(R¹⁵)₂, —NO₂, —CN, and C₁-C₆ alkyl optionally substituted with one or more substituents independently selected from halogen, —OR¹⁵, —N(R¹⁵)₂, —NO₂, and —CN. In certain embodiments, R², R³, R⁴, and R⁵ are independently selected at each occurrence from —N(R¹⁵)₂, —NO₂, —CN, and C₁-C₆ alkyl optionally substituted with one or more substituents independently selected from halogen, —OR¹⁵, —N(R¹⁵)₂, —NO₂, and —CN. In certain embodiments, R², R³, R⁴, and R⁵ are independently selected at each occurrence from —NO₂, —CN, and C₁-C₆ alkyl optionally substituted with one or more substituents independently selected from halogen, —OR¹⁵, —N(R¹⁵)₂, —NO₂, and —CN. In certain embodiments, R², R³, R⁴, and R⁵ are independently selected at each occurrence from —CN, and C₁-C₆ alkyl optionally substituted with one or more substituents independently selected from halogen, —OR¹⁵, —N(R¹⁵)₂, —NO₂, and —CN. In certain embodiments, R², R³, R⁴, and R⁵ are each C₁-C₆ alkyl optionally substituted with one or more substituents independently selected from halogen, —OR¹⁵, —N(R¹⁵)₂, —NO₂, and —CN. In certain embodiments, R², R³, R⁴, and R⁵ are independently selected at each occurrence from halogen, —OR¹⁵, and C₁-C₆ alkyl optionally substituted with one or more substituents independently selected from halogen, —OR¹⁵, and —CN.

In certain embodiments, for a compound or salt selected from any one of Formulas (I), (IIB), (IIC), (IID), and (IIE), R⁷ and R⁸ are independently selected at each occurrence from hydrogen, halogen, —OR¹⁵, —N(R¹⁵)₂, —C(O)R¹⁵, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN. In certain embodiments, R⁷ and R⁸ are independently selected at each occurrence from hydrogen, halogen, —OR¹⁵, —N(R¹⁵)₂, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN. In certain embodiments, R⁷ and R⁸ are independently selected at each occurrence from hydrogen, halogen, —OR¹⁵, —N(R¹⁵)₂, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN.

In certain embodiments, for a compound or salt selected from any one of Formulas (I), (IIB), (IIC), (IID), and (IIE), R⁷ and R⁸ are independently selected at each occurrence from hydrogen, halogen, and C₁-C₆ alkyl optionally substituted with one or more substituents independently selected from halogen, —OR¹⁵, —N(R¹⁵)₂, —NO₂, and —CN. In certain embodiments, R⁷ and R⁸ are independently selected at each occurrence from hydrogen and halogen. In certain embodiments, R⁷ and R⁸ are C₁-C₆ alkyl optionally substituted with one or more substituents independently selected from halogen, —OR¹⁵, —N(R¹⁵)₂, —NO₂, and —CN. In certain embodiments, for a compound or salt selected from any one of Formulas (I), (IIB), (IIC), (ID), and (IIE), each R⁷ and each R⁸ is hydrogen.

In certain embodiments, for a compound or salt of Formula (I) or (IIA), n is independently selected from 0, 1, 2, 3, and 4. In certain embodiments, n is independently selected from 0, 1, 2, and 3. In certain embodiments, n is independently selected from 0, 1, and 2. In certain embodiments, n is independently selected from 0 and 1. In certain embodiments, n is 0.

In certain embodiments, for a compound or salt of any one of Formulas (I), (IIA), (IIB), and (IIC), s is selected from 0, 1, 2, 3, and 4. In certain embodiments, s is selected from 0, 1, 2, and 3. In certain embodiments, s is selected from 0, 1, and 2. In certain embodiments, s is selected from 0 and 1. In certain embodiments, s is 0.

In certain embodiments, for a compound or salt of any one of Formulas (I), (IIA), and (IIB), m is selected from 0, 1, 2, 3, and 4. In certain embodiments, m is selected from 0, 1, 2, and 3. In certain embodiments, m is selected from 0, 1, and 2. In certain embodiments, m is selected from 0 and 1. In certain embodiments, m is 0.

In certain embodiments, for a compound or salt of any one of Formulas (I), (IIA), (IIB), (IIC), and (IID), t is independently selected from 0, 1, 2, 3, and 4. In certain embodiments, t is selected from 0, 1, 2, and 3. In certain embodiments, t is selected from 0, 1, and 2. In certain embodiments, t is selected from 0 and 1. In certain embodiments, t is 0.

In certain embodiments, for a compound or salt of any one of Formulas (I), (IIA), (IIB), (IIC), (IID), (IIE), and (IIF), R⁶, R⁹, R¹¹, R¹², and R¹³ are independently selected from C₁-C₃ alkyl optionally substituted with one or more substituents independently selected from halogen, —OR¹⁵, —SR¹⁵, —N(R¹⁵)₂, —NO₂, and —CN. In certain embodiments, R⁶, R⁹, R¹, R¹², and R¹³ are each C₁ alkyl optionally substituted with one or more substituents independently selected from halogen, —OR¹⁵, —SR¹⁵, —N(R¹⁵)₂, —NO₂, and —CN. In certain embodiments, R⁶, R⁹, R¹, R¹² and R¹³ are each C₂ alkyl optionally substituted with one or more substituents independently selected from halogen, —OR¹⁵, —SR¹⁵, —N(R¹⁵)₂, —NO₂, and —CN. In certain embodiments, R⁶, R⁹, R¹¹, R¹², and R¹³ are each C₃ alkyl optionally substituted with one or more substituents independently selected from halogen, —OR¹⁵, —SR¹⁵, —N(R¹⁵)₂, —NO₂, and —CN.

In certain embodiments, for a compound or salt of any one of Formulas (I), (IIA), (IIB), (IIC), (IID), (IIE), and (IIF), R⁶, R⁹, R¹¹, R¹², and R¹³ are independently selected from methyl optionally substituted with one or more substituents independently selected from halogen, —OR¹⁵, —N(R¹⁵)₂, —NO₂, and —CN. In certain embodiments, R⁶, R⁹, R¹¹, R¹², and R¹³ are independently selected at each occurrence from methyl optionally substituted with one or more substituents independently selected from halogen, —N(R¹⁵)₂, —NO₂, and —CN. In certain embodiments, R⁶, R⁹, R¹¹, R¹², and R¹³ are independently selected at each occurrence from methyl optionally substituted with one or more substituents independently selected from halogen, —NO₂, and —CN. In certain embodiments, R⁶, R⁹, R¹¹, R¹², and R¹³ are independently selected at each occurrence from methyl optionally substituted with one or more substituents independently selected from halogen, and —CN. In certain embodiments, R⁶, R⁹, R¹¹, R¹², and R¹³ are each methyl.

In certain embodiments, for a compound or salt of Formula (I):

R¹ is selected from —C(O)(CR²⁰₂)₁₋₆NR¹², —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)₂;

R¹⁰ is hydrogen;

R², R³, R⁴, and R⁵ are independently selected at each occurrence from halogen, —OR¹⁵, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;

R⁷ and R⁸ are independently selected at each occurrence from hydrogen, halogen, —OR¹⁵, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;

n, s, m, and t are each 0, 1, or 2;

R⁶, R⁹, R¹¹, R¹², and R¹³ are independently selected at each occurrence from C₁-C₆ alkyl optionally substituted with one or more substituents independently selected from halogen, —OR¹⁵, —SR¹⁵, —N(R¹⁵)₂, —NO₂, and —CN;

R¹⁵ is independently selected at each occurrence from hydrogen, and C₁-C₃ alkyl optionally substituted with one or more substituents independently selected from halogen, —CN, —NO₂, —NH₂, —OH, and —OCH₃;

R²⁰ is independently selected at each occurrence from hydrogen, halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —SR²², —S—S—R²², —N(R²²)₂, —N(R²²)₃⁺, —NR²²C(═N)N(R²²)₂, —S(═O)R²², —S(═O)₂R²², —S(═)₂N(R²²)₂, —NR²²S(═O)₂R²², —S—C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

or two R²¹ groups are taken together with the atoms to which they are attached form a heterocycle, optionally substituted with one or more R²⁴;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²³ is selected from:

—C₁₋₆alkylene-(X¹—C₁₋₆alkylene)₁₋₂₄-X¹—C₁₋₆alkyl, wherein X¹ is independently selected at each occurrence from O, S, and NH and wherein R²³ is optionally substituted on a carbon or nitrogen atom with one or more substituents independently selected from R²⁴; and

a 3- to 12-membered heterocycle optionally substituted with one or more substituents independently selected from R²⁴;

R²⁴ is independently selected at each occurrence from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(R²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and

X² is independently selected at each occurrence from O and NH.

In certain embodiments, for a compound or salt of Formula (I):

R¹ is selected from —C(O)(C(R²⁰)₂)₁₋₆C(O)OR²¹ and —C(O)OC(R²⁰)₃;

R¹⁰ is hydrogen;

R², R³, R⁴, and R⁵ are independently selected at each occurrence from halogen, —OR¹⁵, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;

R⁷ and R⁸ are each hydrogen;

n, s, m, and t are each 0, 1, or 2;

R⁶, R⁹, R¹, R¹², and R¹³ are independently selected at each occurrence from C₁-C₃ alkyl optionally substituted with one or more substituents independently selected from halogen, —OR¹⁵, —SR¹⁵, —N(R¹⁵)₂, —NO₂, and —CN;

R¹⁵ is independently selected at each occurrence from hydrogen, and C₁-C₃ alkyl optionally substituted with one or more substituents independently selected from halogen, —CN, —NO₂, —NH₂, —OH, and —OCH₃;

R²⁰ is independently selected at each occurrence from hydrogen, halogen, —NO₂, —CN, —OR²², —SR²², —N(R²²)₂, —P(O)(OR²²)₂, —OP(O)(OR²²)₂; C₁₋₆ alkyl optionally substituted with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle;

R²¹ is selected from hydrogen; and C₁₋₆ alkyl optionally substituted at each occurrence with one or more substituents selected from halogen, —NO₂, —CN, —OR²², —SR²², —N(R²²)₂, and —N(R²²)₃⁺; and

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃.

In certain embodiments, for a compound or salt of Formula (I):

R¹ is selected from —C(O)(CR²⁰₂)₁₋₆NR²¹₂;

R¹⁰ is hydrogen;

R², R³, R⁴, and R⁵ are independently selected at each occurrence from halogen, —OR¹⁵, and C₁-C₆ alkyl optionally substituted with one or more substituents independently selected from halogen, —OR¹⁵, and —CN.;

R⁷ and R⁸ are independently selected at each occurrence from hydrogen, halogen, and C₁-C₆ alkyl optionally substituted with one or more substituents independently selected from halogen, —OR¹⁵, —N(R¹⁵)₂, —NO₂, and —CN;

n, s, m, and t are independently selected from 0 and 1;

R⁶, R⁹, R¹¹, R¹², and R¹³ are independently selected at each occurrence from C₁-C₃ alkyl optionally substituted with one or more substituents independently selected from halogen, —NO₂, and —CN;

R¹⁵ is independently selected at each occurrence from hydrogen, and C₁-C₃ alkyl optionally substituted with one or more substituents independently selected from halogen, —CN, —NH₂, —OH, and —OCH₃;

R²⁰ is independently selected at each occurrence from hydrogen, halogen, —NO₂, —CN, —OR²², —N(R²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂, C₃₋₁₂ carbocycle and 3- to 12-membered heterocycle; wherein each C₃₋₁₂ carbocycle and 3- to 12-membered heterocycle in R²⁰ is independently optionally substituted at each occurrence with one or more substituents selected from halogen, —NO₂, —CN, —OR²², —N(R²²)₂, C₁₋₆ alkyl, C₁₋₆ haloalkyl, C₂₋₆ alkenyl, and C₂₋₆ alkynyl;

R²¹ is selected from hydrogen, —C(O)R²²; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂, C₃₋₁₂ carbocycle and 3- to 12-membered heterocycle; wherein each C₃₋₁₂ carbocycle and 3- to 12-membered heterocycle in R²¹ is independently optionally substituted at each occurrence with one or more substituents selected from halogen, —NO₂, —CN, —OR²², —N(R²²)₂, C₁₋₆ alkyl, C₁₋₆ haloalkyl, C₂₋₆ alkenyl, and C₂₋₆ alkynyl;

or two R²¹ groups are taken together with the atoms to which they are attached form a heterocycle, optionally substituted with one or more R²⁴;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²⁴ is independently selected at each occurrence from halogen, —NO₂, —CN; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂; and C₃₋₁₂ carbocycle and 3- to 12-membered heterocycle, wherein each C₃₋₁₂ carbocycle and 3- to 12-membered heterocycle in R²⁴ is independently optionally substituted at each occurrence with one or more substituents selected from halogen, —NO₂, —CN, C₁₋₆ alkyl, C₁₋₆ haloalkyl, C₂₋₆ alkenyl, and C₂₋₆ alkynyl.

In certain embodiments, for a compound or salt of Formula (I):

R¹ is —C(O)R²³;

R¹⁰ is hydrogen;

R², R³, R⁴, and R⁵ are independently selected at each occurrence from halogen, —OR¹⁵, and C₁-C₆ alkyl optionally substituted with one or more substituents independently selected from halogen, —OR¹⁵, and —CN;

R⁷ and R⁸ are independently selected at each occurrence from hydrogen, halogen, and C₁-C₆ alkyl optionally substituted with one or more substituents independently selected from halogen, —OR¹⁵, —N(R¹⁵)₂, —NO₂, and —CN;

n, s, m, and t are independently selected from 0 and 1;

R⁶, R⁹, R¹¹, R¹², and R¹³ are independently selected at each occurrence from C₁-C₃ alkyl optionally substituted with one or more substituents independently selected from halogen, —NO₂, and —CN;

R¹⁵ is independently selected at each occurrence from hydrogen, and C₁-C₃ alkyl optionally substituted with one or more substituents independently selected from halogen, —CN, —NH₂, —OH, and —OCH₃;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²³ is selected from —C₁₋₆alkylene-(X¹—C₁₋₆alkylene)₁₋₂₄-X¹—C₁₋₆alkyl, wherein X¹ is independently selected at each occurrence from O, S, and NH and wherein R²³ is optionally substituted on a carbon or nitrogen atom with one or more substituents independently selected from R²⁴;

R²⁴ is independently selected at each occurrence from halogen, —NO₂, —CN, —OR²², —N(R²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —N(R²²)₂, —N(R²²)₃, —NR²²C(═N)N(R²²)₂; and C₃₋₁₂ carbocycle and 3- to 12-membered heterocycle, wherein each C₃₋₁₂ carbocycle and 3- to 12-membered heterocycle in R²⁴ is independently optionally substituted at each occurrence with one or more substituents selected from halogen, —NO₂, —CN, —OR²², —N(R²²)₂, C₁₋₆ alkyl, C₁₋₆ haloalkyl, C₂₋₆ alkenyl, and C₂₋₆ alkynyl.

In certain embodiments, for a compound or salt of Formula (I):

R¹ is —C(O)R²³;

R¹⁰ is hydrogen;

R², R³, R⁴, and R⁵ are independently selected at each occurrence from halogen, —OR¹⁵, and C₁-C₆ alkyl optionally substituted with one or more substituents independently selected from halogen, —OR¹⁵, and —CN;

R⁷ and R⁸ are independently selected at each occurrence from hydrogen, halogen, and C₁-C₆ alkyl optionally substituted with one or more substituents independently selected from halogen, —OR¹⁵, —N(R¹⁵)₂, —NO₂, and —CN;

n, s, m, and t are independently selected from 0 and 1;

R⁶, R⁹, R¹¹, R¹², and R¹³ are independently selected at each occurrence from C₁-C₃ alkyl optionally substituted with one or more substituents independently selected from halogen, —NO₂, and —CN;

R¹⁵ is independently selected at each occurrence from hydrogen, and C₁-C₃ alkyl optionally substituted with one or more substituents independently selected from halogen, —CN, —NH₂, —OH, and —OCH₃;

R²² is independently selected at each occurrence from hydrogen and C₁₋₆ alkyl, wherein C₁₋₆ alkyl may be optionally substituted at each occurrence by halogen, —CN, —NO₂, —OH, —NH₂, and —OCH₃;

R²³ is selected from a 3- to 12-membered heterocycle optionally substituted with one or more substituents independently selected from R²⁴;

R²⁴ is independently selected at each occurrence from halogen, —NO₂, —CN, —OR²²; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂.

In certain embodiments, for a compound or salt of Formula (I):

R¹ is selected from —C(O)(C(R²⁰)₂)₁₋₆C(O)OR²¹;

R¹⁰ is hydrogen;

R², R³, R⁴, and R⁵ are independently selected at each occurrence from halogen, —OR¹⁵, and C₁-C₆ alkyl optionally substituted with one or more substituents independently selected from halogen, —OR¹⁵, and —CN;

R⁷ and R⁸ are independently selected at each occurrence from hydrogen, halogen, and C₁-C₆ alkyl optionally substituted with one or more substituents independently selected from halogen, —OR¹⁵, —N(R¹⁵)₂, —NO₂, and —CN;

n, s, m, and t are independently selected from 0 and 1;

R⁶, R⁹, R¹¹, R¹², and R¹³ are independently selected at each occurrence from C₁-C₃ alkyl optionally substituted with one or more substituents independently selected from halogen, —NO₂, and —CN;

R¹⁵ is independently selected at each occurrence from hydrogen, and C₁-C₃ alkyl optionally substituted with one or more substituents independently selected from halogen, —CN, —NH₂, —OH, and —OCH₃;

R²⁰ is independently selected at each occurrence from hydrogen, halogen, —NO₂, —CN, —OR²², —N(R²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —N(R²²)₂, —N(R²²)₃, —NR²²C(═N)N(R²²)₂;

R²¹ is selected from hydrogen; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂; and C₃₋₁₂ carbocycle and 3- to 12-membered heterocycle, wherein each C₃₋₁₂ carbocycle and 3- to 12-membered heterocycle in R²¹ is independently optionally substituted at each occurrence with one or more substituents selected from halogen, —NO₂, —CN, —OR²², —N(R²²)₂, C₁₋₆ alkyl, C₁₋₆ haloalkyl, C₂₋₆ alkenyl, and C₂₋₆ alkynyl; and

R²² is independently selected at each occurrence from hydrogen; and C₁₋₆ alkyl, C₃₋₁₂ carbocycle and 3- to 12-membered heterocycle, each of which may be optionally substituted at each occurrence by halogen, —CN, —NO₂, —OH, —NH₂, and —OCH₃;

In certain embodiments, for a compound or salt of Formula (I):

R¹ is —C(O)(C(R²⁰)₂)₁₋₆C(O)OR²¹;

R¹⁰ is hydrogen;

R², R³, R⁴, and R⁵ are independently selected at each occurrence from halogen, —OR¹⁵, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;

R⁷ and R⁸ are each hydrogen;

n, s, m, and t are each 0, 1, or 2, such as each of n, s, m, and t are 0;

R⁶, R⁹, R¹¹, R¹², and R¹³ are independently selected at each occurrence from C₁-C₃ alkyl optionally substituted with one or more substituents independently selected from halogen, —OR¹⁵, —SR¹⁵, —N(R¹⁵)₂, —NO₂, and —CN, such as each of R⁶, R⁹, R¹¹, R¹², and R¹³ are CH₃;

R¹⁵ is independently selected at each occurrence from hydrogen, and C₁-C₃ alkyl optionally substituted with one or more substituents independently selected from halogen, —CN, —NO₂, —NH₂, —OH, and —OCH₃;

R²⁰ is independently selected at each occurrence from hydrogen, halogen, —NO₂, —CN, —OR²², —SR²², —N(R²²)₂, —P(O)(OR²²)₂, —OP(O)(OR²²)₂; C₁₋₆ alkyl optionally substituted with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle, such as each R²⁰ is selected from hydrogen and —N(R²²)₂;

R²¹ is selected from hydrogen; and C₁₋₆ alkyl optionally substituted at each occurrence with one or more substituents selected from halogen, —NO₂, —CN, —OR²², —SR²², —N(R²²)₂, and —N(R²²)₃⁺ such as R²¹ is methyl or hydrogen; and

R²² is independently selected at each occurrence from hydrogen; and C₁₋₆ alkyl, C₃₋₁₂ carbocycle and 3- to 12-membered heterocycle, each of which may be optionally substituted at each occurrence by halogen, —CN, —NO₂, —OH, —NH₂, and —OCH₃.

In certain embodiments, the compound is

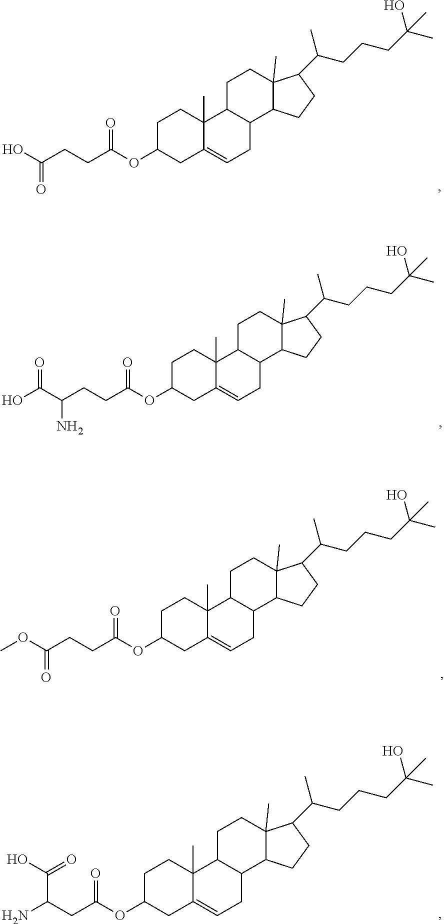

or a salt of any one thereof.

In certain embodiments, the compound is

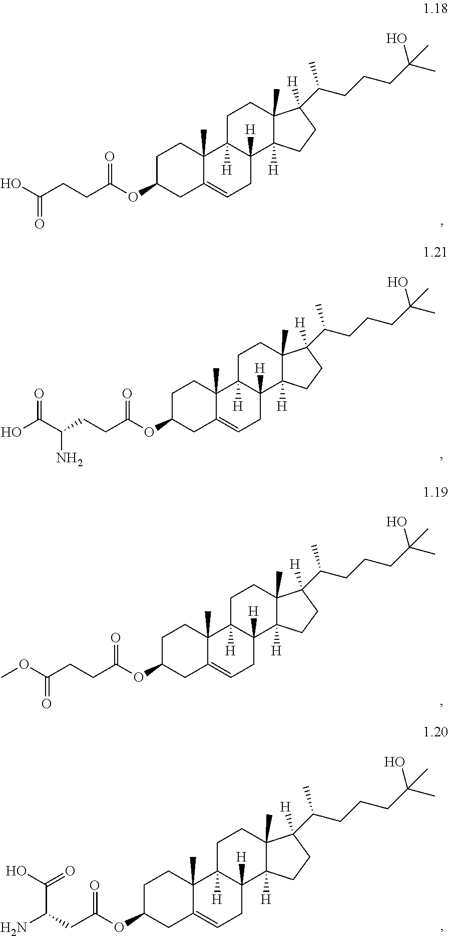

or a salt of any one thereof.

In certain embodiments, the compound is

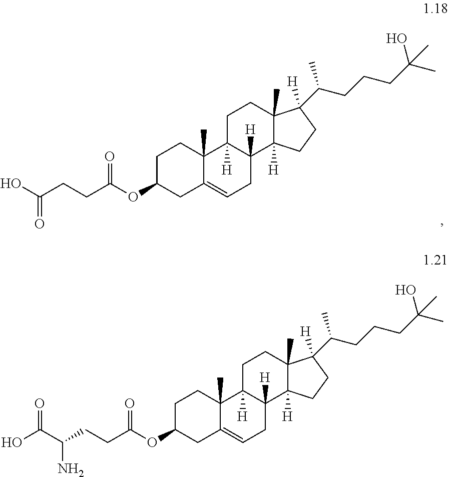

or a salt of any one thereof.

In certain embodiments, for a compound or salt of Formula (I):

R¹ is selected —C(O)OC(R²⁰)₃;

R¹⁰ is hydrogen;

R², R³, R⁴, and R⁵ are independently selected at each occurrence from halogen, —OR¹⁵, and C₁-C₆ alkyl optionally substituted with one or more substituents independently selected from halogen, —OR¹⁵, and —CN;

R⁷ and R⁸ are independently selected at each occurrence from hydrogen, halogen, and C₁-C₆ alkyl optionally substituted with one or more substituents independently selected from halogen, —OR¹⁵, —N(R¹⁵)₂, —NO₂, and —CN;

n, s, m, and t are independently selected from 0 and 1;

R⁶, R⁹, R¹¹, R¹², and R¹³ are independently selected at each occurrence from C₁-C₃ alkyl optionally substituted with one or more substituents independently selected from halogen, —NO₂, and —CN;

R¹⁵ is independently selected at each occurrence from hydrogen, and C₁-C₃ alkyl optionally substituted with one or more substituents independently selected from halogen, —CN, —NH₂, —OH, and —OCH₃;

R²⁰ is independently selected at each occurrence from hydrogen, halogen, —NO₂, —CN, —OR²², —N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²); C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²); and C₃₋₁₂ carbocycle and 3- to 12-membered heterocycle, wherein each C₃₋₁₂ carbocycle and 3- to 12-membered heterocycle in R²⁰ is independently optionally substituted at each occurrence with one or more substituents selected from halogen, —NO₂, —CN, —OR²², —N(R²²)₂; and

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃.

In certain embodiments, for a compound or salt of Formula (I):

R¹ is selected from —C(O)OC(R²⁰)₃;

R¹⁰ is hydrogen;

R², R³, R⁴, and R⁵ are independently selected at each occurrence from halogen, —OR¹⁵, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;

R⁷ and R⁸ are each hydrogen;

n, s, m, and t are each 0, 1, or 2, such as each of n, s, m, and t are 0;

R⁶, R⁹, R¹¹, R¹², and R¹³ are independently selected at each occurrence from C₁-C₃ alkyl optionally substituted with one or more substituents independently selected from halogen, —OR¹⁵, —SR¹⁵, —N(R¹⁵)₂, —NO₂, and —CN, such as each of R⁶, R⁹, R¹¹, R¹², and R¹³ are methyl;

R¹⁵ is independently selected at each occurrence from hydrogen, and C₁-C₃ alkyl optionally substituted with one or more substituents independently selected from halogen, —CN, —NO₂, —NH₂, —OH, and —OCH₃;

R²⁰ is independently selected at each occurrence from hydrogen, halogen, —NO₂, —CN, —OR²², —SR²², —N(R²²)₂, —P(O)(OR²²)₂, —OP(O)(OR²²)₂; C₁₋₆ alkyl optionally substituted with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle, such as each R²⁰ is independently hydrogen or C₁₋₆ alkyl optionally substituted with one or more substituents selected from halogen, —NO₂, —CN, —OR²², —SR²², —N(R²²)₂, —N(R²²)₃⁺; and

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃; such as each R²² is methyl.

In certain embodiments, the compound is

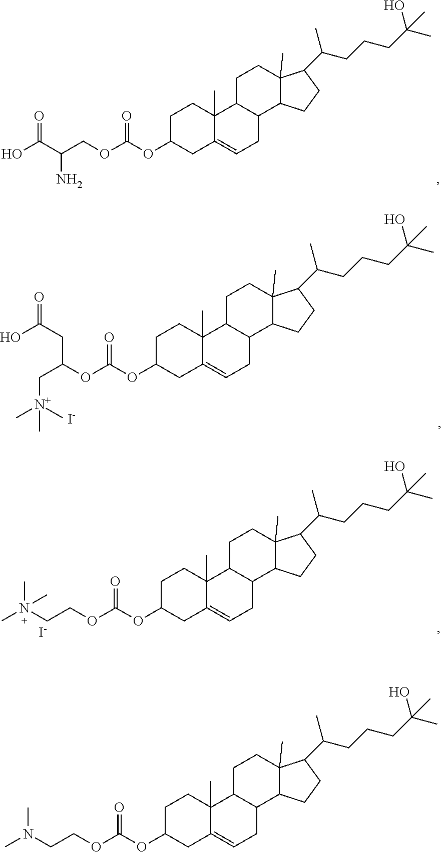

or a salt, or alternative salt in the case of the quaternary amine, of any one thereof.

In certain embodiments, the compound is

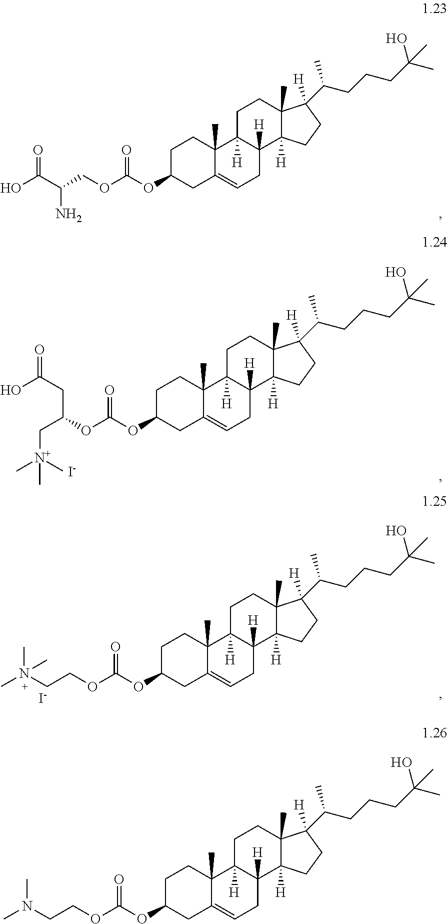

or a salt, or alternative salt in the case of the quaternary amine, of any one thereof.

In certain embodiments, the compound is

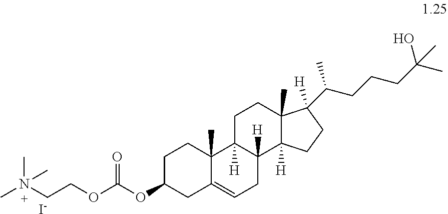

or another salt thereof, e.g., a chloride salt, or bromide salt.

In certain embodiments, for a compound or salt of Formula (I):

R¹ is —P(═O)(X²R²¹)₂;

R¹⁰ is hydrogen;

R², R³, R⁴, and R⁵ are independently selected at each occurrence from halogen, —OR¹⁵, and C₁-C₆ alkyl optionally substituted with one or more substituents independently selected from halogen, —OR¹⁵, and —CN;

R⁷ and R⁸ are independently selected at each occurrence from hydrogen, halogen, and C₁-C₆ alkyl optionally substituted with one or more substituents independently selected from halogen, —OR¹⁵, —N(R¹⁵)₂, —NO₂, and —CN;

n, s, m, and t are independently selected from 0 and 1;

R⁶, R⁹, R¹¹, R¹², and R¹³ are independently selected at each occurrence from C₁-C₃ alkyl optionally substituted with one or more substituents independently selected from halogen, —NO₂, and —CN;

R¹⁵ is independently selected at each occurrence from hydrogen, and C₁-C₃ alkyl optionally substituted with one or more substituents independently selected from halogen, —CN, —NH₂, —OH, and —OCH₃;

R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂, C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂; and C₃₋₁₂ carbocycle and 3- to 12-membered heterocycle, wherein each C₃₋₁₂ carbocycle and 3- to 12-membered heterocycle in R²¹ is independently optionally substituted at each occurrence with one or more substituents selected from halogen, —NO₂, —CN, —OR²², —N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

or two R²¹ groups are taken together with the atoms to which they are attached form a heterocycle, optionally substituted with one or more R²⁴;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²⁴ is independently selected at each occurrence from halogen, —NO₂, —CN, —OR²², —N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂; and C₃₋₁₂ carbocycle and 3- to 12-membered heterocycle, wherein each C₃₋₁₂ carbocycle and 3- to 12-membered heterocycle in R²⁴ is independently optionally substituted at each occurrence with one or more substituents selected from halogen, —NO₂, —CN, —OR²², —N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and

X² is independently selected at each occurrence from O and NH.

In certain embodiments, for a compound or salt of Formula (I):

R¹ is —CH₂OP(═O)(X²R²¹)₂;

R¹⁰ is hydrogen;

R², R³, R⁴, and R⁵ are independently selected at each occurrence from halogen, —OR¹⁵, and C₁-C₆ alkyl optionally substituted with one or more substituents independently selected from halogen, —OR¹⁵, and —CN;

R⁷ and R⁸ are independently selected at each occurrence from hydrogen, halogen, and C₁-C₆ alkyl optionally substituted with one or more substituents independently selected from halogen, —OR¹⁵, —N(R¹⁵)₂, —NO₂, and —CN;

n, s, m, and t are independently selected from 0 and 1;

R⁶, R⁹, R¹¹, R¹², and R¹³ are independently selected at each occurrence from C₁-C₃ alkyl optionally substituted with one or more substituents independently selected from halogen, —NO₂, and —CN;

R¹⁵ is independently selected at each occurrence from hydrogen, and C₁-C₃ alkyl optionally substituted with one or more substituents independently selected from halogen, —CN, —NH₂, —OH, and —OCH₃;

R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂, C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂; and C₃₋₁₂ carbocycle and 3- to 12-membered heterocycle, wherein each C₃₋₁₂ carbocycle and 3- to 12-membered heterocycle in R²¹ is independently optionally substituted at each occurrence with one or more substituents selected from halogen, —NO₂, —CN, —OR²², —N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

or two R²¹ groups are taken together with the atoms to which they are attached form a heterocycle, optionally substituted with one or more R²⁴;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²⁴ is independently selected at each occurrence from halogen, —NO₂, —CN, —OR²², —N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —N(R²²)₂, —N(R²²)₃⁺, —NR²²C(═N)N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂; and C₃₋₁₂ carbocycle and 3- to 12-membered heterocycle, wherein each C₃₋₁₂ carbocycle and 3- to 12-membered heterocycle in R²⁴ is independently optionally substituted at each occurrence with one or more substituents selected from halogen, —NO₂, —CN, —OR²², —N(R²²)₂, —C(O)R²², —C(O)OR²², —OC(O)R²², —OC(O)OR²², —OC(O)N(R²²)₂, —NR²²C(O)R²², —C(O)N(R²²)₂, ═O, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and

X² is independently selected at each occurrence from O and NH.

In certain embodiments, a compound of the disclosure, such as a compound or salt of any of Formulas (I), (IIA), (IIB), (IIC), (IID), (IIE), (IIF), (IIG), (IIH), (IIJ), (IIK), (IIL), (III), (IV) and (V), has a chemical stability of 350 min or greater, 400 minutes or greater, 450 minutes or greater, 500 minutes or greater, 550 min or greater, 600 minutes or greater or even 650 minutes or greater as evaluated using the chemical stability procedure in EXAMPLE 21 in the Examples section herein. In certain embodiments, a compound of the disclosure, such as a compound or salt of any of Formulas (I), (IIA), (IIB), (IIC), (IID), (IIE), (IIF), (IIG), (IIH), (IIJ), (IIK), (IIL), (III), (IV) and (V), has a plasma stability of 350 min or great, 400 minutes or greater, 450 minutes or greater, 500 minutes or greater, 550 min or greater, 600 minutes or greater or even 650 minutes or greater as evaluated using the plasma stability procedure in EXAMPLE 21 in the Examples section herein.

Any combination of the groups described above for the various variables is contemplated herein. Throughout the specification, groups and substituents thereof can be chosen to provide stable moieties and compounds.

In certain embodiments, the compound or salt according to Formula (I) is selected from:

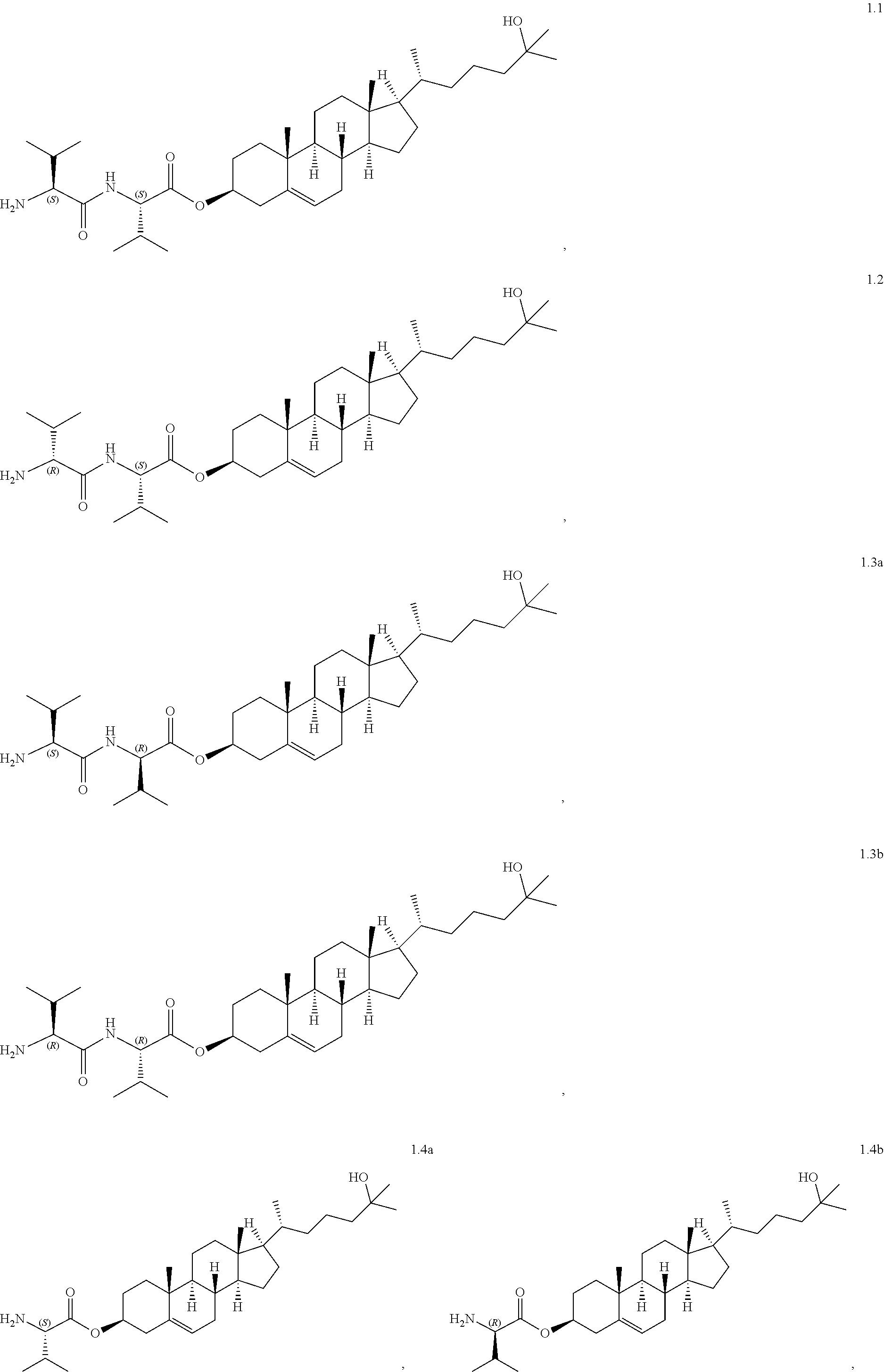

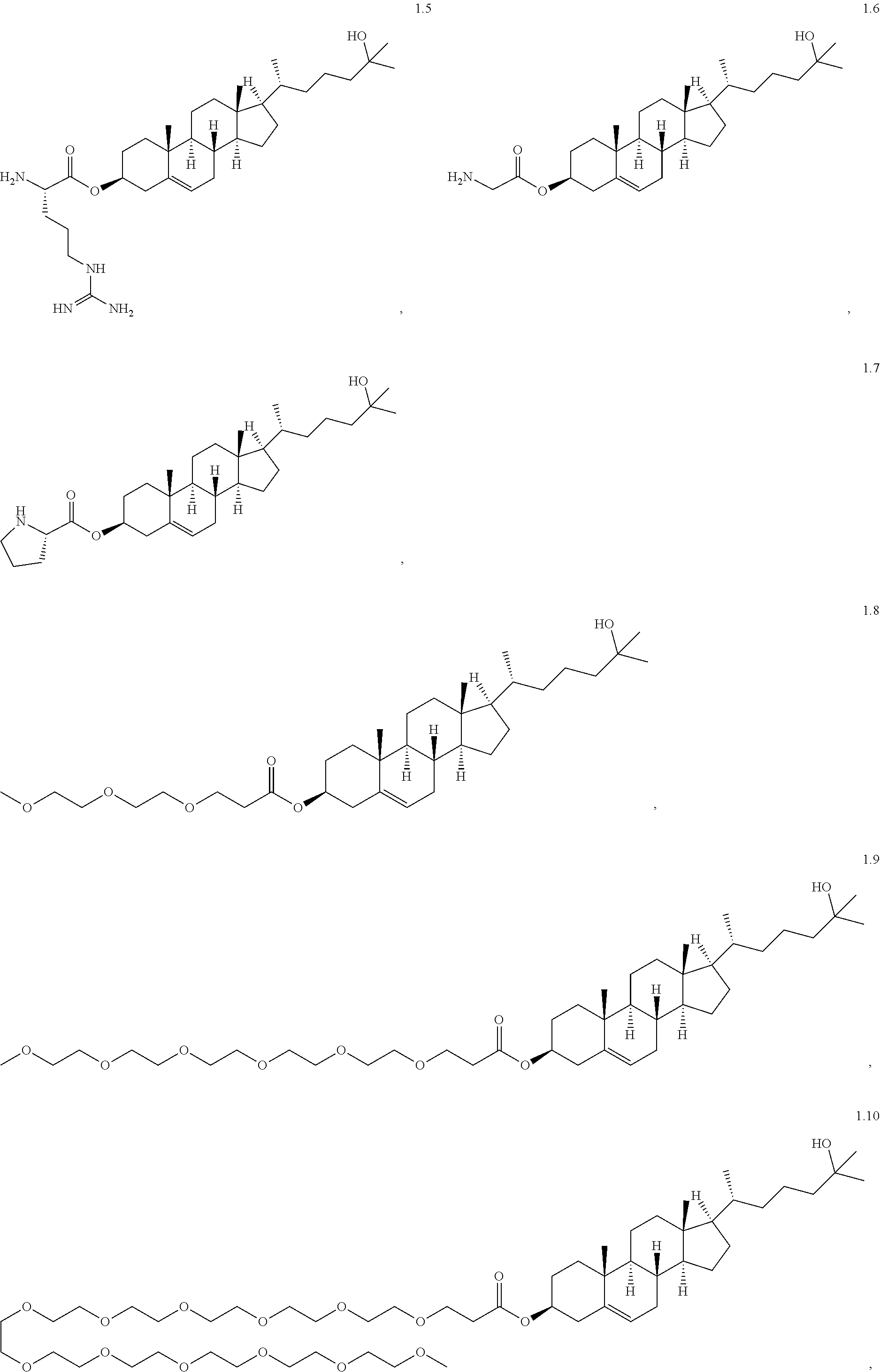

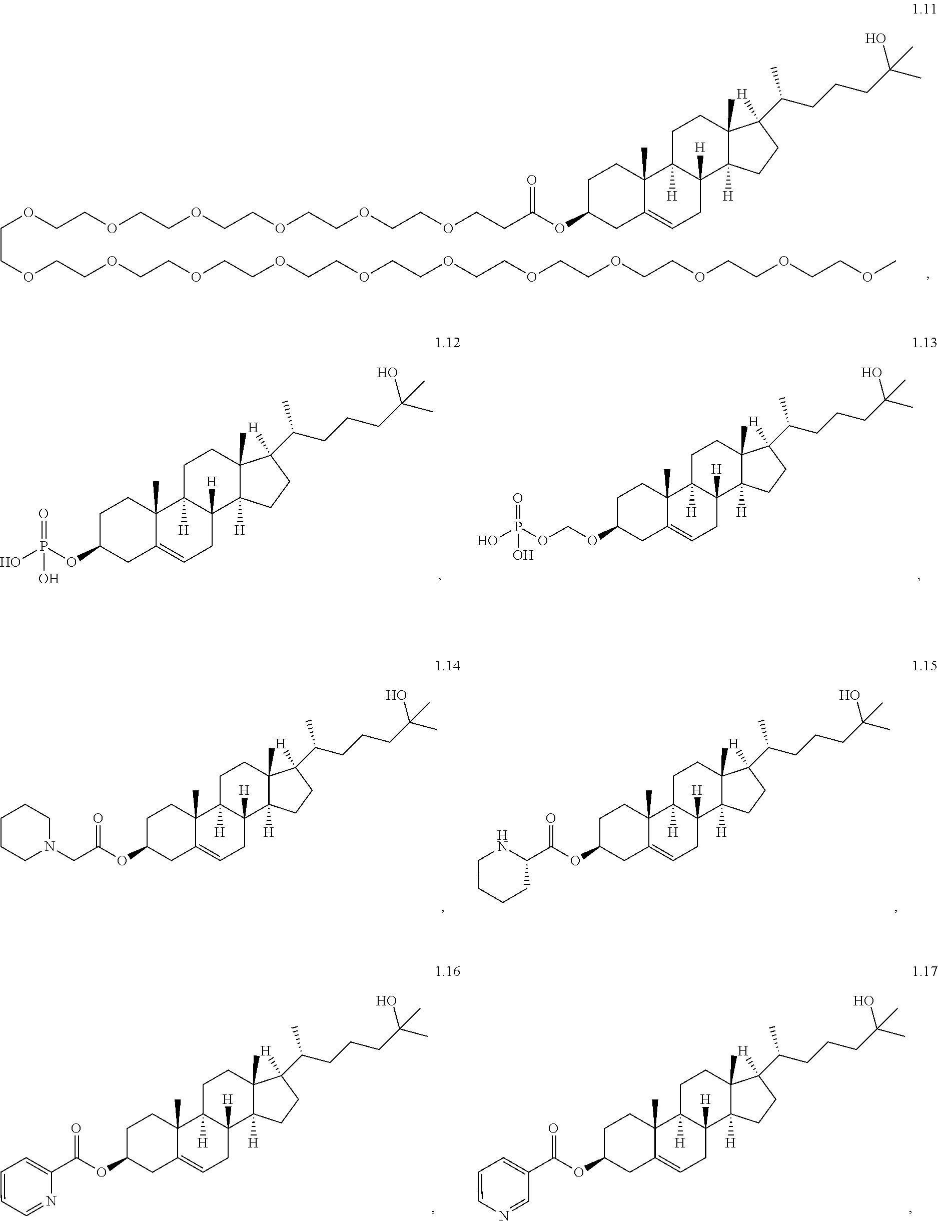

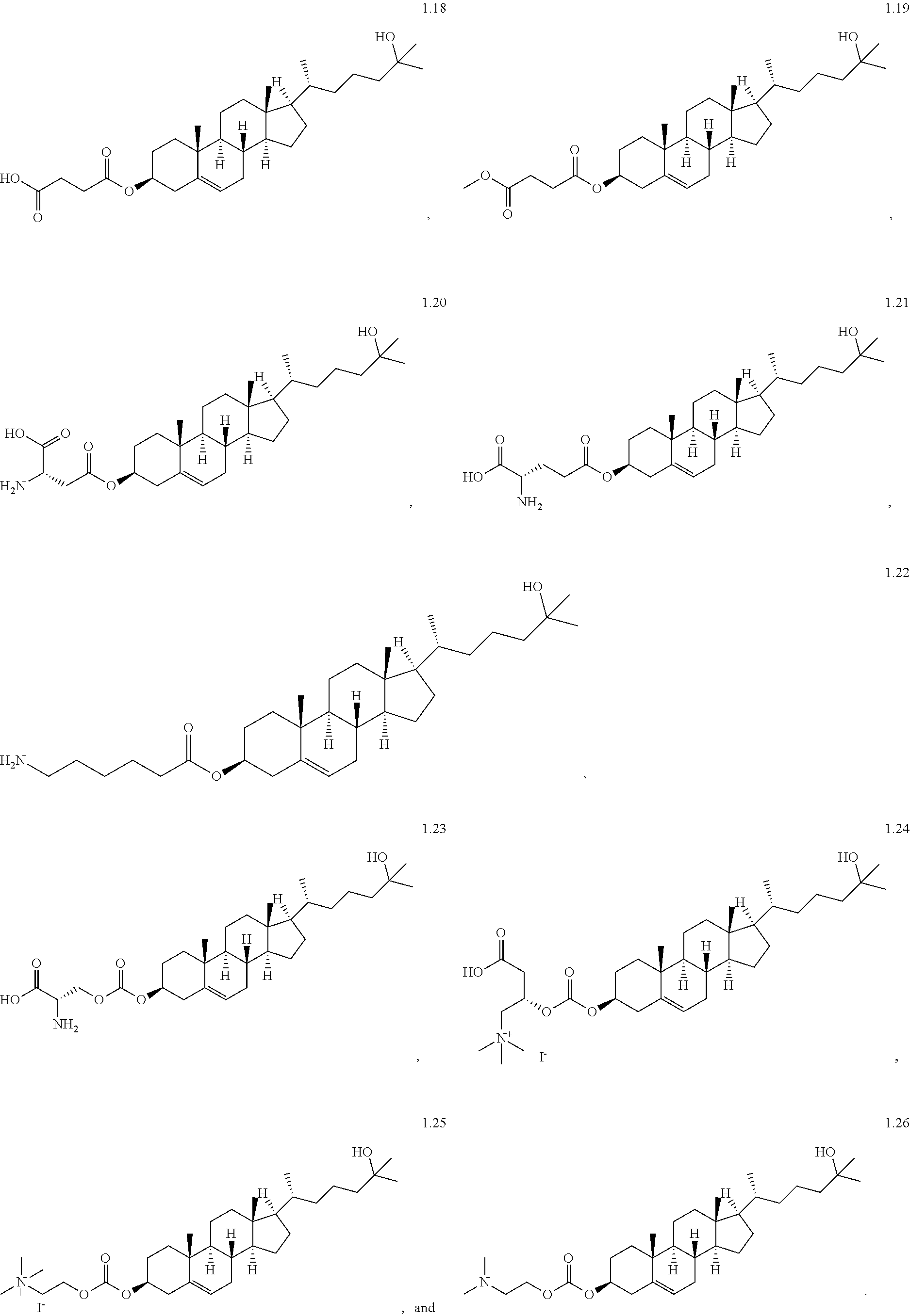

In certain embodiments, the compound or salt of the disclosure is a prodrug or sterol analog with a polarity or solubility enhancing moiety, wherein the sterol selected from:

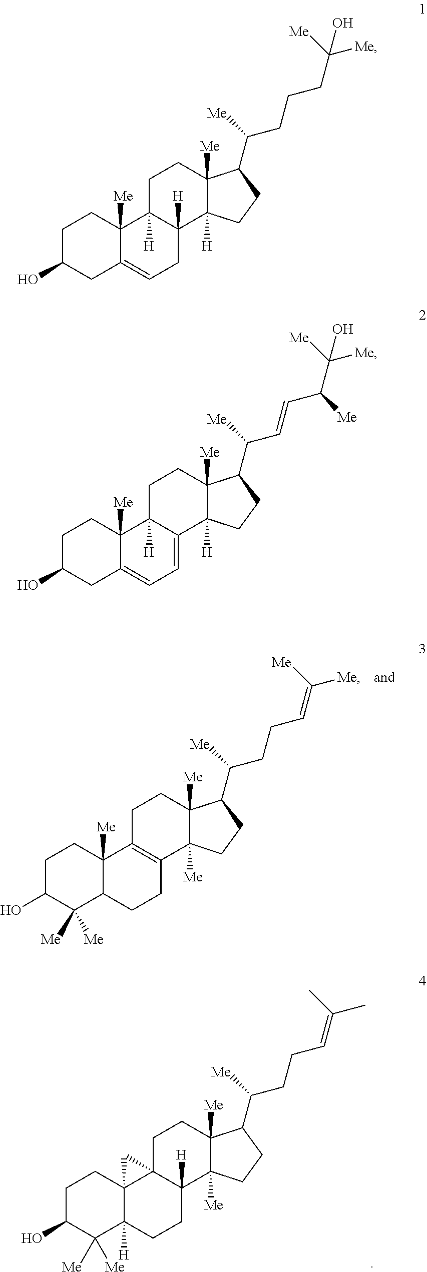

In some embodiments, the compound is of Formula (III):

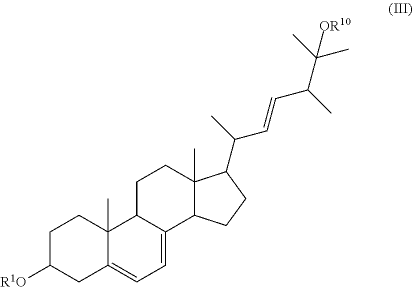

or a salt thereof, wherein:

R¹ and R¹⁰ are independently selected from hydrogen, —C(O)(CR²⁰₂)₁₋₆NR²¹₂, —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)₂, wherein at least one of R¹ and R¹⁰ is not hydrogen;

R²⁰ is independently selected at each occurrence from hydrogen, halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²⁰ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —SR²², —S—S—R²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —S—C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²¹ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

or two R²¹ groups are taken together with the atoms to which they are attached form a heterocycle, optionally substituted with one or more R²⁴;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²³ is selected from:
—C₁₋₆alkylene-(X¹—C₁₋₆alkylene)₁₋₂₄-X—C₁₋₆alkyl, wherein X¹ is independently selected at each occurrence from 0, S, and NH and wherein R²³ is optionally substituted on a carbon or nitrogen atom with one or more substituents independently selected from R²⁴; and
a 3- to 12-membered heterocycle optionally substituted with one or more substituents independently selected from R²⁴;

R²⁴ is independently selected at each occurrence from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²⁴ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and

X² is independently selected at each occurrence from O and NH.

In some embodiments, the compound is of Formula (IV):

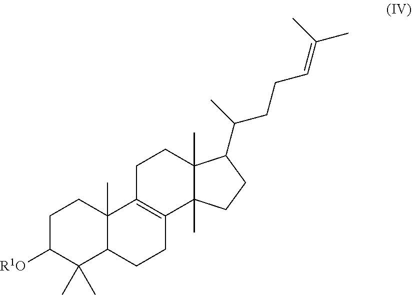

or a salt thereof, wherein:

R¹ is independently selected from hydrogen, —C(O)(CR²⁰₂)₁₋₆NR²¹₂, —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)²;

R²⁰ is independently selected at each occurrence from hydrogen, halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²⁰ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —SR²², —S—S—R²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —S—C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²¹ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

or two R²¹ groups are taken together with the atoms to which they are attached form a heterocycle, optionally substituted with one or more R²⁴;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²³ is selected from:
—C₁₋₆alkylene-(X¹—C₁₋₆alkylene)₁₋₂₄-X¹—C₁₋₆alkyl, wherein X¹ is independently selected at each occurrence from 0, S, and NH and wherein R²³ is optionally substituted on a carbon or nitrogen atom with one or more substituents independently selected from R²⁴; and
a 3- to 12-membered heterocycle optionally substituted with one or more substituents independently selected from R²⁴;

R²⁴ is independently selected at each occurrence from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²⁴ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and

X² is independently selected at each occurrence from O and NH.

In some embodiments, the compound is of Formula (V):

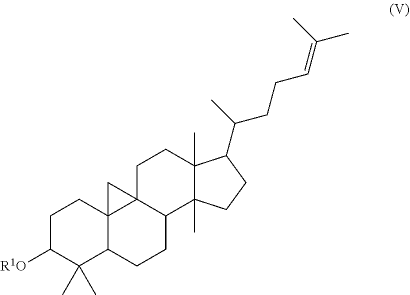

or a salt thereof, wherein:

R¹ is independently selected from hydrogen, —C(O)(CR²⁰₂)₁₋₆NR²¹₂, —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)²;

R²⁰ is independently selected at each occurrence from hydrogen, halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²⁰ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —SR²², —S—S—R²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —S—C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²¹ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;

or two R²¹ groups are taken together with the atoms to which they are attached form a heterocycle, optionally substituted with one or more R²⁴;

R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;

R²³ is selected from:
—C₁₋₆alkylene-(X¹—C₁₋₆alkylene)₁₋₂₄-X¹—C₁₋₆alkyl, wherein X¹ is independently selected at each occurrence from 0, S, and NH and wherein R²³ is optionally substituted on a carbon or nitrogen atom with one or more substituents independently selected from R²⁴; and
a 3- to 12-membered heterocycle optionally substituted with one or more substituents independently selected from R²⁴;

R²⁴ is independently selected at each occurrence from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle in R²⁴ is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and

X² is independently selected at each occurrence from O and NH.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans). When the compounds described herein can form tautomers, and unless specified otherwise, all tautomeric forms are also intended to be included. The term “geometric isomer” refers to E or Z geometric isomers (e.g., cis or trans) of an alkene double bond.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. Unless otherwise stated, chemical structures depicted herein are intended to include structures which are different tautomers of the structures depicted. For example, the chemical structure depicted with an enol moiety also includes the keto tautomer form of the enol moiety. The exact ratio of the tautomers depends on several factors, including physical state, temperature, solvent, and pH. Some examples of tautomeric equilibrium include:

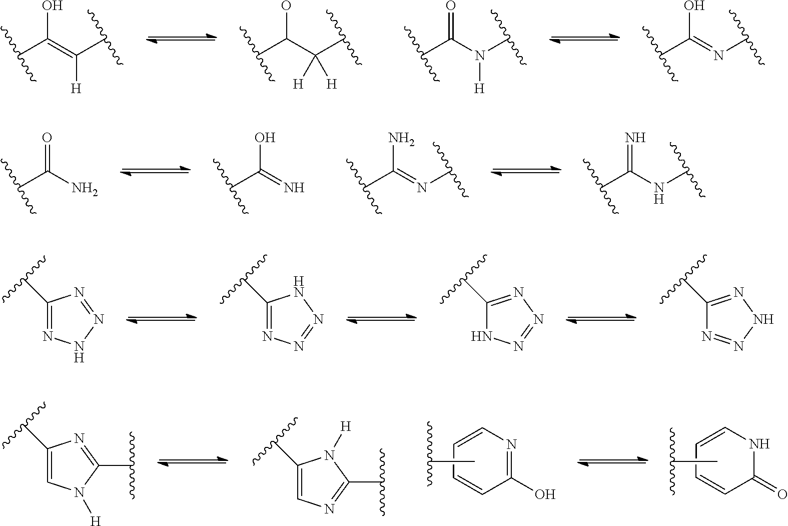

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

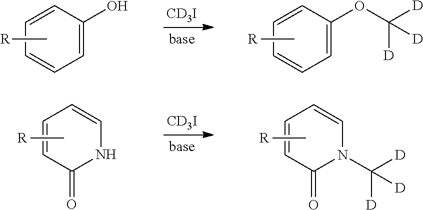

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

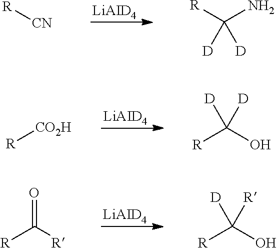

Deuterium gas and palladium catalyst are employed to reduce unsaturated carbon-carbon linkages and to perform a reductive substitution of aryl carbon-halogen bonds as illustrated, by way of example only, in the reaction schemes below.

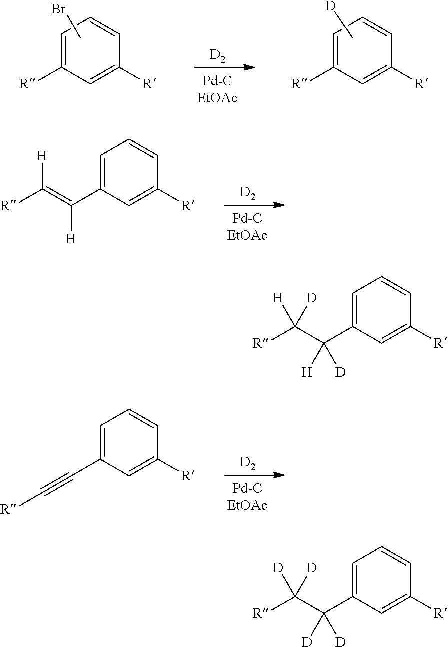

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

In certain embodiments, a compound or salt of any one of Formulas (I), (IIA), (IIB), (IIC), (IID), (IIE), (IIF), (IIG), (IIH), (IIJ), (IIK), (IIL), (III), (IV) and (V) may have improved bioavailability relative to be easier to administer than 25-hydroxycholesterol. In certain embodiments, a compound or salt of any one of Formulas (I), (IIA), (IIB), (IIC), (IID), (IIE), (IIF), (IIG), (IIH), (IIJ), (IIK), (IIL), (III), (IV) and (V) may have enhanced cell permeability relative to 25-hydroxycholesterol. In certain embodiments, a compound or salt of any one of Formulas (I), (IIA), (IIB), (IIC), (IID), (IIE), (IIF), (IIG), (IIH), (IIJ), (IIK), (IIL), (III), (IV) and (V) may have improved solubility in pharmaceutical formulations relative to 25-hydroxycholesterol. In certain embodiments, a compound or salt of any one of Formulas (I), (IIA), (IIB), (IIC), (IID), (IIE), (IIF), (IIG), (IIH), (IIJ), (IIK), (IIL), (III), (IV) and (V) may have increased lipophilicity relative to 25-hydroxycholesterol. In certain embodiments, a compound or salt of any one of (I), (IIA), (IIB), (IIC), (IID), (IIE), (IIF), (IIG), (IIH), (IIJ), (IIK), (IIL), (III), (IV) and (V) may have increased effective water solubility relative to 25-hydroxycholesterol.

Unless otherwise stated, a compound or salt of any one of Formulas (I), (IIA), (IIB), (IIC), (IID), (IIE), (IIF), (IIG), (IIH), (IIJ), (IIK), (IIL), (III), (IV) and (V) includes tautomers, and compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

Methods and formulations described herein include the use of amorphous forms as well as crystalline forms (also known as polymorphs). In addition, the compounds described herein can exist in unsolvated as well as solvated forms with pharmaceutically acceptable solvents such as water, ethanol, and the like. The solvated forms of the compounds and salts presented herein are also considered to be disclosed herein.

A compound or salt of any one of Formulas (I), (IIA), (IIB), (IIC), (IID), (IIE), (IIF), (IIG), (IIH), (IIJ), (IIK), (IIL), (III), (IV) and (V) may in some cases exist as diastereomers, enantiomers, or other stereoisomeric forms. The compounds and salts presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

### Pharmaceutical Formulations

In certain embodiments, a compound or salt of any one of Formulas (I), (IIA), (IIB), (IIC), (IID), (IIE), (IIF), (IIG), (IIH), (IIJ), (IIK), (IIL), (III), (IV), and (V) is used for the treatment of an ophthalmic disorder such as cataracts or presbyopia. A formulation administered to the eye may be administered by injection, for example, by intravitreal or intracameral injection. A formulation administered to the eye may be administered topically, for example, with an ointment, cream, or eye drop.

In other embodiments, a compound or salt of any one of Formulas (I), (IIA), (IIB), (IIC), (IID), (IIE), (IIF), (IIG), (IIH), (IIJ), (IIK), (IIL), (III), (IV), and (V) with low aqueous solubility may preferentially be formulated with an agent that enhances aqueous solubility. For example, in certain embodiments, the formulations of the disclosure are aqueous formulations for topical administration, wherein the aqueous formulation comprises a solubilizing agent, e.g., a 3-cyclodextrin, to enhance solubility of a compound of salt of the disclosure.

A compound or salt of any one of Formulas (I), (IIA), (IIB), (IIC), (IID), (IIE), (IIF), (IIG), (IIH), (IIJ), (IIK), (IIL), (III), (IV), and (V) can be present in a formulation of the invention at a concentration of, for example, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. A compound or salt of any one of Formulas (I), (IIA), (IIB), (IIC), (ID), (IIE), (IIF), (IIG), (IIH), (IIJ), (IIK), (IIL), (III), (IV), and (V) may be present in a composition within a range of concentrations, the range being defined by an upper and lower value selected from any of the preceding concentrations. For example, a compound or salt of any one of Formulas (I), (IIA), (IIB), (IIC), (IID), (IIE), (IIF), (IIG), (IIH), (IIJ), (IIK), (IIL), (III), (IV), and (V) may be present in the formulation at a concentration of from about 1 nM to about 100 mM, about 10 nM to about 10 mM, about 100 nM to about 1 mM, about 500 nM to about 1 mM, about 1 mM to about 50 mM, about 10 mM to about 40 mM, about 20 mM to about 35 mM, or about 20 mM to about 30 mM. In certain embodiments, an aqueous formulation of the disclosure comprises at least 90 wt % water, such as at least 91 wt %, at least 92 wt %, at least 93 wt %, at least 94 wt %, at least 95 wt %, at least 96 wt %, at least 97 wt %, at least 97.5% at least 98 wt %, at least 98.5%, at least 99 wt %, or even at least 99.5% of water.

In certain embodiments, a formulation, such as an aqueous suspension, of the compounds and salts of any one of Formulas (I), (IIA), (IIB), (IIC), (IID), (IIE), (IIF), (IIG), (IIH), (IIJ), (IIK), (IIL), (III), (IV), and (V) further comprises one or more physiologically acceptable carriers including excipients and auxiliaries which facilitate processing of the pharmaceutical agent into preparations which are used pharmaceutically. Proper formulation is dependent upon the route of administration chosen. A summary of pharmaceutical formulations is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, a formulation, such as an aqueous solution, of the compounds and salts of any one of Formulas (I), (IIA), (IIB), (IIC), (IID), (IIE), (IIF), (IIG), (IIH), (IIJ), (IIK), (IIL), (III), (IV), and (V), further comprises a solubilizing agent. In certain embodiments, a compound or salt of any one of Formulas (I), (IIA), (IIB), (IIC), (IID), (IIE), (IIF), (IIG), (IIH), (IIJ), (IIK), (IIL), (III), (IV), and (V) exhibits low aqueous solubility and the addition of a solubilizing agent enhances the solubility of the compound or salt. Solubilizing agents of the disclosure include, for example, host molecules of inclusions complexes such as cyclodextrins. In some embodiments 3-cyclodextrins are preferred. An example of a suitable β-cyclodextrin includes, for example, (2-hydroxylpropyl)-β-cyclodextrin. In certain embodiments, the formulation comprises from about 2 wt % to about 15 wt % of a solubilizing agent, about 3 wt % to about 12 wt %, about 4 wt % to about 10 wt %, about 5 wt % to about 10 wt %, or about 6 wt % to about 10 wt % of a solubilizing agent, e.g., a cyclodextrin. In certain embodiments, the formulation is an aqueous solution comprising a solubilizing agent, such as a β-cyclodextrin. In preferred embodiments, a formulation for topical administration to the eye comprises a solubilizing agent such as a cyclodextrin.

Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical formulations are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical formulation can be in dosage unit form such as tablet, capsule, granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

Methods for the preparation of compositions comprising the compounds or salts of any one of Formulas (I), (IIA), (IIB), (IIC), (IID), (IIE), (IIF), (IIG), (IIH), (IIJ), (IIK), (IIL), (III), (IV), and (V), can include formulating the compounds with one or more inert, pharmaceutically acceptable excipients. Liquid compositions include, for example, solutions in which a compound is dissolved, emulsions comprising a compound, or a solution containing liposomes, micelles, or nanoparticles comprising a compound as disclosed herein. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, elixir, nanosuspension, microsuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, anti-adherents, anti-static agents, surfactants, antioxidants, coloring agents, flavouring agents, plasticizers, preservatives, suspending agents, emulsifying agents, plant cellulosic material and spheronization agents, and any combination thereof. Non-limiting examples of types of formulations that can be used in a method of the invention include an aqueous solution, an ointment, an aqueous suspension, and an oil in water emulsion.

Pharmaceutical formulations can be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration. Pharmaceutical formulations suitable for topical administration include liquid or semi-liquid preparations suitable for penetration through the skin to the site of inflammation such as gels, liniments, lotions, creams, ointments or pastes, and drops suitable for administration to the eye, ear or nose.

### Combination Therapy

A compound or salt of any one of Formulas (I), (IIA), (IIB), (IIC), (IID), (IIE), (IIF), (IIG), (IIH), (IIJ), (IIK), (IIL), (III), (IV), and (V), or the pharmaceutically acceptable salts thereof, can be administered in combination with one or more therapeutic agents.

A compound or salt of any one of Formulas (I), (IIA), (IIB), (IIC), (IID), (IIE), (IIF), (IIG), (IIH), (IIJ), (IIK), (IIL), (III), (IV), and (V), or a pharmaceutically acceptable salt thereof, may be co-administered with a second therapeutic agent, wherein the compound or salt of any one of Formulas (I), (IIA), (IIB), (IIC), (IID), (IIE), (IIF), (IIG), (IIH), (III), (IIK), (IIL), (III), (IV), and (V), or a pharmaceutically acceptable salt thereof, and the second therapeutic agent modulate different aspects of the disease, disorder or condition being treated, thereby providing a greater overall benefit than administration of either therapeutic agent alone.

Different therapeutically-effective dosages of a compound or salt of any one of Formulas (I), (IIA), (IIB), (IIC), (IID), (IIE), (IIF), (IIG), (IIH), (IIJ), (IIK), (IIL), (III), (IV), and (V), can be utilized in formulating a pharmaceutical formulation or in treatment regimens when the compounds disclosed herein are administered in combination with one or more additional agent. Therapeutically-effective dosages of drugs and other agents for use in combination treatment regimens is optionally determined by means similar to those set forth hereinabove for the therapeutic agents themselves. Furthermore, the methods of prevention/treatment described herein encompasses the use of metronomic dosing, for example, providing more frequent, lower doses in order to minimize toxic side effects. A combination treatment regimen can encompass treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. The disclosure also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

For combination therapies described herein, dosages of the co-administered compounds vary depending on the type of co-drug employed, on the specific drug employed, on the disease or condition being treated and so forth. In certain embodiments, when co-administered with one or more other therapeutic agents, a compound or salt of any one of Formulas (I), (IIA), (IIB), (IIC), (ID), (IIE), (IIF), (IIG), (IIH), (IIJ), (IIK), (IIL), (III), (IV), and (V) is administered either simultaneously with the one or more other therapeutic agents, or sequentially.

A compound or salt of any one of Formulas (I), (IIA), (IIB), (IIC), (IID), (IIE), (IIF), (IIG), (IIH), (IIJ), (IIK), (IIL), (III), (IV), and (V), or the pharmaceutically acceptable salts thereof, as well as combination therapies, may be administered before, during or after the occurrence of a disease or condition, and the timing of administering the composition containing a compound varies. A compound or salt of any one of Formulas (I), (IIA), (IIB), (IIC), (IID), (IIE), (IIF), (IIG), (IIH), (IIJ), (IIK), (IIL), (III), (IV), and (V) can be used as a prophylactic and may be administered continuously to subjects with a propensity to develop conditions or diseases in order to prevent the occurrence of the disease or condition. A compound or salt of any one of Formulas (I), (IIA), (IIB), (IIC), (ID), (IIE), (IIF), (IIG), (IIH), (IIJ), (IIK), (IIL), (III), (IV), and (V) and compositions thereof may be administered to a subject during or as soon as possible after the onset of the symptoms. a compound or salt of any one of Formulas (I), (IIA), (IIB), (IIC), (IID), (IIE), (IIF), (IIG), (IIH), (IIJ), (IIK), (IIL), (III), (IV), and (V) may be administered as soon as is practicable after the onset of a disease or condition is detected or suspected, and for a length of time necessary for the treatment of the disease. The length required for treatment may vary, and the treatment length is adjusted to suit the specific needs of each subject. For example, a compound or salt of any one of Formulas (I), (IIA), (IIB), (IIC), (IID), (IIE), (IIF), (IIG), (IIH), (IIJ), (IIK), (IIL), (III), (IV), and (V) or a formulation containing the compound or salt can be administered for at least 2 weeks, about 1 month to about 5 years.

In certain embodiments, a compound or salt of any one of Formulas (I), (IIA), (IIB), (IIC), (IID), (IIE), (IIF), (IIG), (IIH), (IIJ), (IIK), (IIL), (III), (IV), and (V) can be used in combination with lipoic acid, an ester of lipoic acid such as the choline ester of lipoic acid, Humanin peptides, 3,6-dibromocarbazole piperazine derivatives of 2-propanol, Ku70 peptides, 4-phenylsulphanyl-phenylamine derivatives, IDN-6556, Anilinoquinazolines (AQZs), Nicotinyl aspartyl ketones, M826|M867, Pifithrin-α, INO-1001, FR255595, 3AB, NU1025, INH2BP, GP16150 and PJ34. In certain embodiments, a compound or salt of any one of Formulas (I), (IIA), (IIB), (IIC), (IID), (IIE), (IIF), (IIG), (IIH), (IIJ), (IIK), (IIL), (III), (IV), and (V) may be used in combination with lipoic acid, an ester of lipoic acid such as the choline ester of lipoic acid, glutathione, ascorbate, vitamin E, Uric acid, melatonin, vitamin C, Tirilazad, NXY-059, (R)-2-((5-(1,2-dithiolan-3-yl)pentanoyl)oxy)-N,N,N-trimethylethan-1-aminium, carotenes and ubiquinol. In certain embodiments, a compound of the disclosure is administered in combination, e.g., in the same formulation or in separate formulations, with lipoic acid or the choline ester of lipoic acid.

### Dosing

The compositions and methods of the present disclosure may be utilized to treat an individual in need thereof. In certain embodiments, the individual is a mammal such as a human, or a non-human mammal. When administered to an animal, such as a human, the composition or the pharmaceutical agent, is preferably administered as a pharmaceutical formulation comprising, for example, a compound or salt of any one of Formulas (I), (IIA), (IIB), (IIC), (IID), (IIE), (IIF), (IIG), (IIH), (IIJ), (IIK), (IIL), (III), (IV) and (V) and a pharmaceutically acceptable carrier or excipient.

In practicing the methods of treatment or use provided herein, therapeutically-effective amounts of a compound or salt of any one of Formulas (I), (IIA), (IIB), (IIC), (IID), (IIE), (IIF), (IIG), (IIH), (IIJ), (IIK), (IIL), (III), (IV), and (V) are administered in pharmaceutical formulations to a subject having a disease or condition to be treated. A therapeutically-effective amount can vary widely depending on the severity of the disease, the age and relative health of the subject, the potency of the compounds used, and other factors. Subjects can be, for example, human subjects such as elderly adults, adults, adolescents, pre-adolescents, children, toddlers, infants, or neonates. A subject can be a patient. Subjects can be non-human animals, for example, chimpanzees, apes, monkeys, cattle, horses, sheep, goats, swine, rabbits, dogs, and cats, rats, mice and guinea pigs.

In certain embodiments, formulations of the disclosure are used to treat ophthalmic diseases through administration to an eye of a subject. The formulations can be delivered to the eye topically through cream, ointment or liquid drop formulation. The formulation can be delivered to the eye through injection. Injectable solutions can be delivered directly into the anterior chamber, sclera, vitreous humor, cornea, crystalline lens, or surrounding tissue. The compositions can also be delivered as an intraocular perfusate.

### Methods of the Disclosure

The disclosure provides compounds and formulations for use in reducing or preventing alpha-crystallin protein aggregation. The aggregation of alpha-crystallin has been implicated in a variety of diseases of which the compounds and formulations described herein may be used to treat or prevent. Such diseases include, for example, cataracts, nuclear sclerosis, presbyopia, neurological diseases, Alexander disease, Creutzfeldt-Jacob disease, Alzheimer's disease, and Parkinson's disease.

In certain embodiments, the methods provided herein can be used to treat a disease or a condition that would benefit from inhibiting, reducing the likelihood of, or reversing the aggregation of alpha-crystallin. A compound or salt of any one of Formulas (I), (IIA), (IIB), (IIC), (IID), (IIE), (IIF), (IIG), (IIH), (IIJ), (IIK), (IIL), (III), (IV), and (V) can be used as pharmacological chaperones of alpha-crystallin.

The methods provided herein can be used to treat, for example, a vision disorder such as cataract, age-related cataract, diabetic cataract, a cataract associated with surgery, a cataract resulting from radiation, a cataract resulting from a genetic illness, a cataract resulting from an infection, a cataract resulting from medication, or a hereditary form of cataract with early onset.

Vision disorders, as discussed herein, refer to disordered vision that may be associated with aberrant aggregation of crystallin proteins in the lens of the eye. The aberrant aggregation of crystallin proteins may be the primary factor resulting in the vision disorder or may be one of a plurality of mechanisms resulting in the vision disorder. Vision disorders of the disclosure include, but are not limited to, cataract, such as nuclear cataract, cortical cataract, posterior capsular cataract, congenital cataract, early-onset hereditary cataract, metabolic (diabetic) cataract, secondary cataract, blunt traumatic cataract, penetrating traumatic cataract, post-vitrectomy cataract, radiation-induced cataract; and presbyopia, such as incipient presbyopia, functional presbyopia, absolute presbyopia, premature presbyopia or nocturnal presbyopia.

The methods of the invention can also be used to treat disease caused by an alphaA- or alphaB-crystallin mutation. The mutation in alphaA- or alphaB-crystallin can lead to hereditary cataract. Examples of alphaA-crystallin mutations include, but are not limited to, R54C, G98R, R21L, R116C, and W9X. Examples of alphaB-crystallin mutations include, but are not limited to, 150delA (aB184), D140N, P20S, D109H and R120G. In some instances, the alphaB-crystallin mutation is R120G or D109H.

In certain embodiments, a compound or salt of any one of Formulas (I), (IIA), (IIB), (IIC), (IID), (IIE), (IIF), (IIG), (IIH), (IIJ), (IIK), (IIL), (III), (IV), and (V) is used to treat a subject with a vision disorder, such as cataract or presbyopia. In certain embodiments, a compound or salt of any one of Formulas (I), (IIA), (IIB), (IIC), (IID), (IIE), (IIF), (IIG), (IIH), (IIJ), (IIK), (IIL), (III), (IV), and (V) may be used to treat cataract of a subject, such as nuclear cataract, cortical cataract, posterior capsular cataract, congenital cataract, secondary cataract, traumatic cataract, radiation cataract. In certain embodiments, a subject has one or more symptoms of cataract, such as clouded vision, blurred vision, dim vision, trouble seeing at night, sensitivity to light and glare, need for brighter light for reading and other activities, seeing “halos” around lights, frequent changes in eyeglasses or contact lens prescription, fading or yellowing of colors, and double vision in a single eye.

In certain embodiments, a compound or salt of any one of Formulas (I), (IIA), (IIB), (IIC), (IID), (IIE), (IIF), (IIG), (IIH), (IIJ), (IIK), (IIL), (III), (IV), and (V) may be used to treat presbyopia of a subject, such as incipient presbyopia, functional presbyopia, absolute presbyopia, premature presbyopia or nocturnal presbyopia. In certain embodiments, the subject has one or more symptoms of presbyopia, such as decreased focusing ability for near objects, eyestrain, difficulty reading fine print, fatigue while reading or looking at an illuminated screen, difficulty seeing clearly up close, less contrast when reading print, need for brighter and more direct light for reading, needing to hold reading material further away in order to see it clearly, or headaches, especially headaches when using near vision. In some embodiments the subject does not have a cataract in an eye afflicted with presbyopia.

In certain embodiments, the subject has a vision disorder in one eye. In certain embodiments, the subject has a vision disorder in both eyes.

The subject of the disclosure can be any vertebrate animal. In some preferred embodiments the subject is a human. The subject may be of any age. In some embodiments the subject may be between 25 and 100 years of age, or between 40 and 100 years of age, or between 50 and 100 years of age. The subject may be over 1 year of age, over 2 years of age, over 5 years of age, over 10 years of age, over 18 years of age, over 20 years of age, over 25 years of age, over 30 years of age, over 35 years of age, over 40 years of age, over 45 years of age, over 50 years of age, over 60 years of age, over 70 years of age, over 80 years of age or over 90 years of age. The subject may be 25 years of age or older.

The methods provided herein can be used to treat a disease or a condition that would benefit from reducing the likelihood of or reversing the aggregation of alpha-crystallin by administering an effective amount of at least one of the compounds or formulations described herein. An effective amount can be an amount that reduces or inhibits the aggregation of alpha-crystallin. In certain embodiments, a compound or salt of any one of Formulas (I), (IIA), (IIB), (IIC), (IID), (IIE), (IIF), (IIG), (IIH), (IIJ), (IIK), (IIL), (III), (IV), and (V) reduces alpha-crystallin aggregation in an eye by about 1% to about 100%, about 1% to about 90%, about 1% to about 80%, about 1% to about 70%, about 10% to about 50%, about 20% to about 40%, about 50% to about 90% or between 60% to about 95% relative to a pretreatment value for alpha-crystallin aggregation.

A compound or salt of any one of Formulas (I), (IIA), (IIB), (IIC), (IID), (IIE), (IIF), (IIG), (IIH), (IIJ), (IIK), (IIL), (III), (IV), and (V) may be used to inhibit the aggregation of alpha-crystallin by at least about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% when compared to a pre-treatment level or a level observed in biologically matched control subject or specimen that was not administered said compounds. A compound or salt of any one of Formulas (I), (IIA), (IIB), (IIC), (IID), (IIE), (IIF), (IIG), (IIH), (IIJ), (IIK), (IIL), (III), (IV), and (V) may inhibit the aggregation of the alpha-crystallin by between 1% and 100%, between 5% and 90%, between 10% and 80%, between 20% and 50%, between 50% and 95%, between 60% and 99% or between 40% and 70% when compared to a pre-treatment level or a level observed in biologically matched control subject or specimen that was not administered said compounds.

Alpha-crystallin aggregation in the lens may be measured with, for example, in vivo dynamic light scattering, light scattering assays, electron microscopy, centrifugation protein solubility assays, filter trap protein solubility assays, thioflavin T-fluorescence assays, high performance liquid chromatography, gel-permeation chromatography, size exclusion chromatography, anti-amyloid antibody assays. In certain embodiments, exemplary methods of the disclosure for measuring alpha-crystallin aggregation in the lens are described in: K. Dierks et al, SPIE Vol. 2330 Lasers in Ophthalmology 11, 112-121 (1994); R. Ansari, Journal of Biomedical Optics January/February, Vol. 9, No. 1, 22-37 (2004); and X. Pei et al, Br J Ophthalmol 92, 1471-1475 (2008), the contents of each of which are incorporated by reference herein.

A compound or salt of any one of Formulas (I), (IIA), (IIB), (IIC), (IID), (IIE), (IIF), (IIG), (IIH), (IIJ), (IIK), (IIL), (III), (IV), and (V) can inhibit cataract formation by at least about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% when compared to the level observed in biologically matched control subject or specimen that was not administered said compounds.

In certain embodiments, prior to treatment, the subject has experienced a loss of near vision. The subject may have experienced a loss of near vision which first occurred when the subject was 25 years of older. The subject may have been about 25 to 50 years old, such as about 25 to 40 years old, such as about 25 to 35 years old when the subject experienced a loss of near vision. The subject may have been diagnosed, e.g., diagnosed by a medical practitioner, as suffering a loss of near vision, or may self-identify as suffering a loss of near vision.

To test the efficacy of a compound or salt of any one of Formulas (I), (IIA), (IIB), (IIC), (IID), (IIE), (IIF), (IIG), (IIH), (III), (IIK), (IIL), (III), (IV), and (V), a bioanalytical method may be employed to allow for pharmacokinetic studies. The bioanalytical method may be sensitive at a scale of about 15 nM of the compound in plasma, and about 20 nM of the compound in ocular tissues. In certain embodiments, the exposure of a compound or salt of any one of Formulas (I), (IIA), (IIB), (IIC), (ID), (IIE), (IIF), (IIG), (IIH), (III), (IIK), (IIL), (III), (IV), and (V) may be measured in the lens of the rabbits which displays slow diffusion because the lens is a protein rich, dense tissue area in the anterior of the eye. Ciliary process levels may be used as a measure of exposure of the compound in the lens. In certain embodiments, the exposure of a compound or salt of any one of Formulas (I), (IIA), (IIB), (IIC), (IID), (IIE), (IIF), (IIG), (IIH), (IIJ), (IIK), (IIL), (III), (IV), and (V) may be measured in the cornea of rabbits. To get to the cornea, a compound or salt of any one of Formulas (I), (IIA), (IIB), (IIC), (IID), (IIE), (IIF), (IIG), (IIH), (IIJ), (IIK), (IIL), (III), (IV), and (V) may pass through the cornea, or sclera, to access the internal structures of the eye. In certain embodiments, the exposure of a compound or salt of any one of Formulas (I), (IIA), (IIB), (IIC), (IID), (IIE), (IIF), (IIG), (IIH), (III), (IIK), (IIL), (III), (IV), and (V) may be measured in the retina of rabbits, which can display fast diffusion because the retina is a lipid-rich, soft tissue area in the back of the eye. In certain embodiments, the exposure of a compound or salt of any one of Formulas (I), (IIA), (IIB), (IIC), (IID), (IIE), (IIF), (IIG), (IIH), (IIJ), (IIK), (IIL), (III), (IV), and (V) may be measured in the ciliary bodies of rabbits, which can display fast diffusion because the ciliary body is lipid-rich, soft tissue in the anterior of the eye.

A compound or salt of any one of Formulas (I), (IIA), (IIB), (IIC), (IID), (IIE), (IIF), (IIG), (IIH), (IIJ), (IIK), (IIL), (III), (IV), and (V) may demonstrate 10% or more increased aqueous solubility relative to 25-hydroxycholesterol, wherein the compound or salt of any one of Formulas (I), (IIA), (IIB), (IIC), (IID), (IIE), (IIF), (IIG), (IIH), (IIJ), (IIK), (IIL), (III), (IV), and (V) and 25-hydroxycholesterol are each subjected to the steps of: a) weigh an excess of sample into an Eppendorf tube; b) add 0.5 mL of water for injection to the Eppendorf tube; c) incubate the Eppendorf tube at 37° C. for 6 h shaking at 200 rpm; d) centrifuge the Eppendorf tube for 10 min at 13,500 rpm; e) pipette 250 μL of liquid and filter through 0.45 m disposable syringe filter; f) collect the filtrate and dilute further with water for injection; g) measure the absorbance of the filtrate by UV spectroscopy; h) calculate solubility in mg/mL; and compare the solubility in step h. of the compound or salt of any one of Formulas (I), (IIA), (IIB), (IIC), (IID), (IIE), (IIF), (IIG), (IIH), (IIJ), (IIK), (IIL), (III), (IV), and (V) with the solubility in step h. of 25-hydroxycholesterol. For example, if 25-hydroxycholesterol demonstrates an aqueous solubility of 10 mg/mL, then the compound or salt of any one of Formulas (I), (IIA), (IIB), (IIC), (IID), (IIE), (IIF), (IIG), (IIH), (IIJ), (IIK), (IIL), (III), (IV), and (V) may demonstrate an aqueous solubility of about 11 mg/mL, about 12 mg/mL, about 13 mg/mL, about 14 mg/mL, or about 15 mg/mL. In some embodiments, the compound or salt of any one of Formulas (I), (IIA), (IIB), (IIC), (IID), (IIE), (IIF), (IIG), (IIH), (IIJ), (IIK), (IIL), (III), (IV), and (V) may demonstrate an aqueous solubility that is about 10% or more, 20% or more, 30% or more, 40% or more, or 50% or more, from 10% to 80%, from 10% to 70%, from 10% to from 60%, or from 10% to 50% greater than the aqueous solubility of 25-hydroxycholesterol.

A compound or salt of any one of Formulas (I), (IIA), (IIB), (IIC), (IID), (IIE), (IIF), (IIG), (IIH), (IIJ), (IIK), (IIL), (III), (IV), and (V) may demonstrate 10% or more increased cellular uptake than 25-hydroxycholesterol, wherein the compound or salt of any one of Formulas (I), (IIA), (IIB), (IIC), (ID), (IIE), (IIF), (IIG), (IIH), (IIJ), (IIK), (IIL), (III), (IV), and (V) and 25-hydroxycholesterol are each subjected to the steps of: a) incubate a sample with cultured primary rabbit corneal cells in DPBS for 10 minutes; b) wash the cells three times with ice-cold HEPES buffer; c) lyse the cells overnight with 1 mL 0.05% (w/v) Triton X-100 in 1 N NaOH at room temperature; d) transfer 500 μL aliquots from each well to scintillation vials containing 5 mL scintillation cocktail; e) subject the sample to liquid scintillation spectrophotometry using a scintillation counter; f) normalize the rate of uptake to the protein content of each well; and g) measure the amount of protein in the cell lysate using bovine serum albumin as the standard; and compare the measured amount of protein in the cell lysate in step g. of the compound or salt of any one of Formulas (I), (IIA), (IIB), (IIC), (IID), (IIE), (IIF), (IIG), (IIH), (IIJ), (IIK), (IIL), (III), (IV), and (V) with the measured amount of protein in the cell lysate in step g. of 25-hydroxycholesterol. For example, if 25-hydroxycholesterol demonstrates a cellular uptake of 10 nM, then the compound or salt of any one of Formulas (I), (IIA), (IIB), (IIC), (IID), (IIE), (IIF), (IIG), (IIH), (IIJ), (IIK), (II), (III), (IV), and (V) may demonstrate a cellular uptake of about 11 nM, about 12 nM, about 13 nM, about 14 nM, or about 15 nM. In some embodiments, the compound or salt of any one of Formulas (I), (IIA), (IIB), (IIC), (IID), (IIE), (IIF), (IIG), (IIH), (IIJ), (IIK), (II), (III), (IV), and (V) may demonstrate a cellular uptake of about 10% or more, 20% or more, 30% or more, 40% or more, or 50% or more, from 10% to 80%, from 10% to 70%, from 10% to 60%, or from 10% to 50% greater than the cellular uptake of 25-hydroxycholesterol.

A compound or salt of any one of Formulas (I), (IIA), (IIB), (IIC), (IID), (IIE), (IIF), (IIG), (IIH), (IIJ), (IIK), (IIL), (III), (IV), and (V) may demonstrate 10% or more increased corneal diffusion than 25-hydroxycholesterol. For example, if 25-hydroxycholesterol demonstrates a corneal diffusion of 100 nM, then the compound or salt of any one of Formulas (I), (IIA), (IIB), (IIC), (ID), (IIE), (IIF), (IIG), (IIH), (IIJ), (IIK), (IIL), (III), (IV), and (V) may demonstrate a corneal diffusion of about 110 nM, about 120 nM, about 130 nM, about 140 nM, or about 150 nM. In some embodiments, the compound or salt of any one of Formulas (I), (IIA), (IIB), (IIC), (ID), (IIE), (IIF), (IIG), (IIH), (IIJ), (IIK), (IIL), (III), (IV), and (V) may demonstrate a corneal diffusion of about 10% or more, 20% or more, 30% or more, 40% or more, or 50% or more, from 10% to 80%, from 10% to 70%, from 10% to 60%, or from 10% to 50% greater than the corneal diffusion of 25-hydroxycholesterol.

### EXAMPLES

### Example 1: General Experimental Procedure

Analytical TLC was performed on Merck silica gel 60 F₂₅₄ aluminum-backed plates. Compounds were visualized by UV light and/or stained with either I₂ or potassium permanganate solution followed by heating. Flash column chromatography was performed on silica gel. ¹H-NMR spectra were recorded on a Bruker Avance-400 MHz spectrometer with a BBO (Broad Band Observe) and BBFO (Broad Band Fluorine Observe) probe. Chemical shifts (6) are expressed in parts per million (ppm) downfield by reference to tetramethylsilane as the internal standard. Splitting patterns are designated as s (singlet), d (doublet), m (multiplet) and br s (broad singlet). Coupling constants (J) are given in hertz (Hz).

### Example 2: Preparation of Compound 1.4a

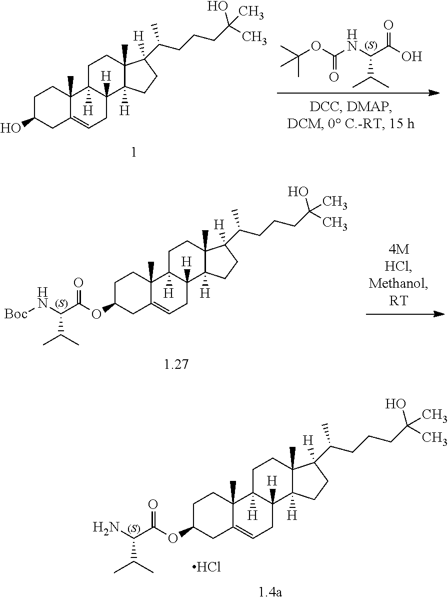

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-L-valinate (1.27)

To a stirred solution of (tert-butoxycarbonyl)-L-valine (108 mg, 0.498 mmol) in dichloromethane (DCM) (5 mL) was added 4-dimethylamino pyridine (DMAP) (91 mg, 0.747 mmol), dicyclohexyl carbodiimide (DCC) (153 mg, 0.747 mmol) at 0° C. followed by addition of a solution of 25-hydroxy cholesterol 1 (100 mg, 0.249 mmol) in DCM (2.0 mL). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (20 mL), saturated solution of NaHCO₃ (20 mL) and water (20 mL) sequentially. The organic layer was dried over anhydrous Na₂SO₄, filtered and concentrated under reduced pressure to get crude. The crude was purified over flash column (Silica 12 g, redisep), eluted with 20% EtOAc in hexane to give pure compound 1.27 (120 mg, Yield: 54%) as off white solid. ¹H-NMR (400 MHz, CDCl₃): δ 5.37 (s, 1H), 5.01-5.03 (m, 1H), 4.65-4.68 (m, 1H), 4.16-4.18 (m, 1H), 2.30-2.32 (m, 2H), 2.10-2.20 (m, 1H), 1.94-1.99 (m, 2H), 1.85-1.88 (m, 4H), 1.52-1.60 (m, 2H), 1.33-1.45 (m, 15H), 1.17-1.20 (m, 10H), 1.09-1.16 (m, 4H), 1.01-1.06 (m, 5H), 0.88-0.97 (m, 11H) and 0.67 (s, 3H). LCMS: 602.4 (M+H)⁺, 99.76%.

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl L-valinate hydrochloride salt (1.4a)

To an ice-cold solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-L-valinate 1.27 (120 mg, 0.199 mmol) in methanol (10 mL) was added 4M HCl in dioxane (5 mL) and resulting mixture stirred at RT for 2 h. After completion of reaction (TLC monitoring), the resulting reaction mixture was evaporated under reduced pressure to get crude residue, which was triturated with diethyl ether (20 mL×2 times) to get desired compound 1.4a (100 mg, Yield: 93%) as off white solid. ¹H-NMR (400 MHz, DMSO-d₆): δ 8.35 (s, 3H), 5.38 (s, 1H), 4.61-4.64 (m, 1H), 4.02 (br s, 1H), 3.87 (d, J=5.4 Hz, 1H), 2.32-2.38 (m, 3H), 2.14-2.17 (m, 2H), 1.81-1.96 (m, 6H), 1.53-1.55 (m, 1H), 1.42-1.48 (m, 4H), 1.22-1.33 (m, 5H), 1.10-1.19 (m, 7H), 0.89-1.05 (m, 18H) and 0.65 (m, 3H). LCMS: 502.4 (M+H)⁺, 98.47%.

### Example 3: Preparation of Compound 1.7

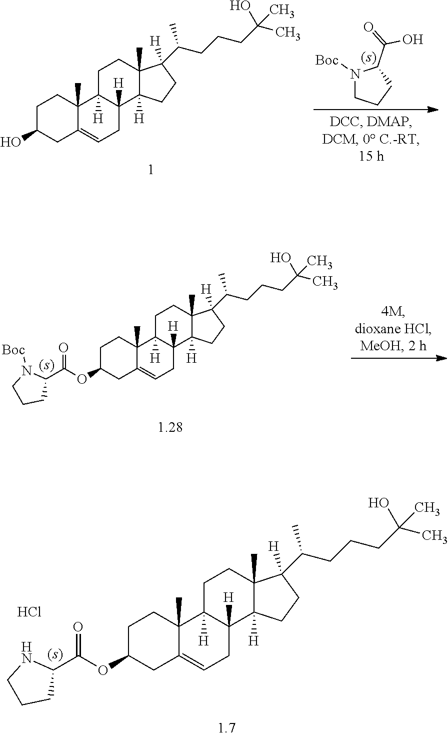

### Preparation of 1-(tert-butyl) 2-((3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl) (S)-pyrrolidine-1,2-dicarboxylate (1.28)

To an ice-cold solution of (tert-butoxycarbonyl)-L-proline (53 mg, 0.248 mmol) in DCM (5 mL) was added DMAP (45 mg, 0.372 mmol), DCC (76 mg, 0.372 mmol) followed by addition of 25-hydroxy cholesterol 1 (50 mg, 0.124 mmol) in DCM (2.0 mL). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (20 mL), saturated solution of NaHCO₃ (20 mL) and water (20 mL) sequentially. The organic layer was dried over anhydrous Na₂SO₄, filtered and concentrated under reduced pressure to get crude. The crude was purified over flash column (Silica 12 g, redisep), eluted with 25% EtOAc in hexane to give pure compound 1.28 (50 mg, Yield: 67%) as off white solid. LCMS: 600.6 (M+H)⁺, 97.82%.

### Preparation (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl L-prolinate hydrochloride salt (1.7)

To a stirred solution of 1-(tert-butyl) 2-((3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl) (S)-pyrrolidine-1,2-dicarboxylate 1.28 (50 mg, 0.083 mmol) in methanol (5 mL) was added 4M HCl in dioxane (2 mL) and resulting mixture stirred at RT for 2 h. After completion of reaction (TLC monitoring), the resulting reaction mixture was evaporated under reduced pressure to get crude residue, which was triturated with diethyl ether (20 mL×2 times) to get desired compound 1.7 (35 mg, Yield: 78%) as off white solid. ¹H-NMR (400 MHz, DMSO-d₆): δ 9.31 (br s, 2H), 5.37 (s, 1H), 4.58 (s, 1H), 4.33 (t, J=8.0 Hz, 1H), 4.01 (s, 1H), 3.24-3.30 (m, 2H), 2.23-2.32 (m, 5H), 1.85-1.96 (m, 10H), 1.61-1.64 (m, 4H), 1.33-1.53 (m, 12H), 1.19-1.33 (m, 9H), 1.09-1.14 (m, 3H), and 0.68 (s, 3H). LCMS: 500.4 (M+H)+, 92.43%.

### Example 4: Preparation of Compound 1.14

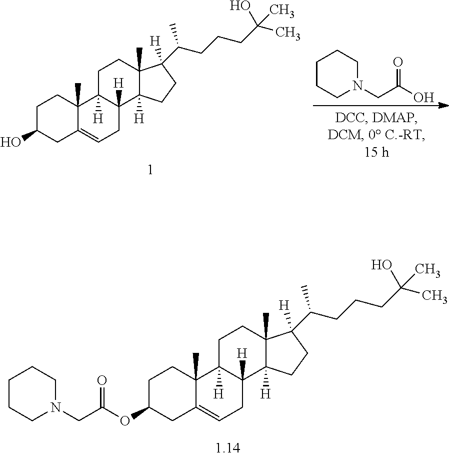

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl 2-(piperidin-1-yl)acetate (1.14)

To an ice-cold solution of 2-(piperidin-1-yl) acetic acid (35 mg, 0.248 mmol) in DCM (5 mL) was added DMAP (45 mg, 0.372 mmol), DCC (76 mg, 0.372 mmol) followed by addition of a solution of 25-hydroxy cholesterol 1 (50 mg, 0.124 mmol) in DCM (2.0 mL). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (20 mL), saturated solution of NaHCO₃ (20 mL) and water (20 mL) sequentially. The organic layer was dried over anhydrous Na₂SO₄, filtered and concentrated under reduced pressure to get crude. The crude was purified twice over flash column (silica 12 g, Redisep), eluted with 25% EtOAc in hexane and further purified over prep-HPLC column chromatography (method attached below) to get desired product 1.14 (20 mg, Yield: 30%) as white solid. ¹H-NMR (400 MHz, DMSO-d6): δ 5.37 (s, 1H), 4.68 (s, 1H), 3.20 (s, 2H), 2.65 (s, 3H), 2.34 (d, J=8.0 Hz, 2H), 1.85-2.02 (m, 5H), 1.37-1.70 (m, 21H), 0.92-1.25 (m, 19H) and 0.67 (s, 3H). LCMS: 528.4 (M+H)⁺, 99.43%.

### Example 5: Preparation of Compound 1.15

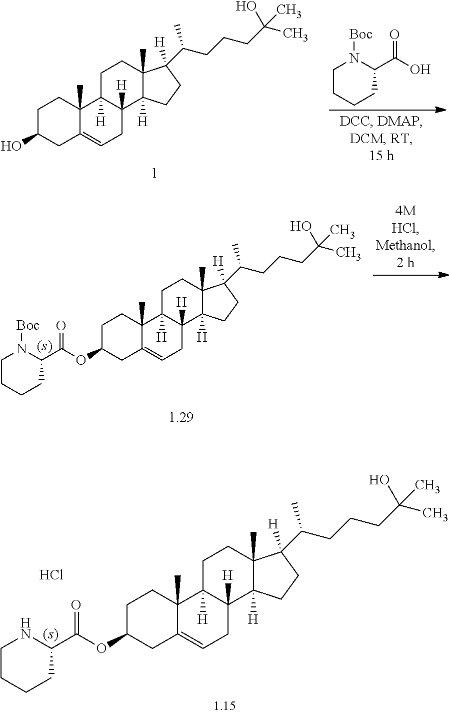

### Preparation of 1-(tert-butyl) 2-((3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl) (S)-piperidine-1,2-dicarboxylate (1.29)

To a solution of 25-hydroxy cholesterol 1 (50 mg, 0.124 mmol) in DCM (5 mL) was added (S)-1-(tert-butoxycarbonyl)piperidine-2-carboxylic acid (56 mg, 0.248 mmol), DMAP (45 mg, 0.372 mmol) and DCC (76 mg, 0.372 mmol). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (20 mL), saturated solution of NaHCO₃ (20 mL) and water (20 mL) sequentially. The organic layer was dried over anhydrous Na₂SO₄, filtered and concentrated under reduced pressure to get crude. The crude was purified over flash column (Silica 12 g, redisep), eluted with 20% EtOAc in hexane to give pure compound 1.29 (40 mg, Yield: 55%) as off white solid. ¹H-NMR (400 MHz, CDCl₃): δ 5.3 (s, 1H), 4.5-4.8 (m, 2H), 3.98 (br s, 1H), 2.22-2.35 (m, 2H), 1.85-2.02 (m, 4H), 1.37-1.64 (m, 22H), 0.67-1.25 (m, 28H), 0.68 (s, 3H). LCMS: 614.6 (M+H)⁺, 91.66%.

### Preparation (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (S)-piperidine-2-carboxylate hydrochloride salt (1.15)

To an ice-cold solution of 1-(tert-butyl) 2-((3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl) (S)-piperidine-1,2-dicarboxylate 1.29 (55 mg, 0.089 mmol) in methanol (5 mL) was added 4M HCl in dioxane (2 mL) and resulting mixture stirred at RT for 2 h. After completion of reaction (TLC monitoring), the resulting reaction mixture was evaporated under reduced pressure to get crude residue, which was triturated with diethyl ether (15 mL×2 times) to get desired compound 1.15 (42 mg, Yield: 85%) as white solid. ¹H-NMR (400 MHz, DMSO-d₆): δ 10.2 (br s, 1H), 9.40 (br s, 1H), 5.38 (s, 1H), 4.88 (s, 1H), 4.01 (s, 1H), 3.5 (s, 1H), 3.17 (s, 1H), 2.08-2.35 (m, 3H), 1.73-2.09 (m, 13H), 1.34-1.46 (m, 10H), 0.92-1.28 (m, 20H) and 0.67 (s, 3H). LCMS: 514.4 (M+H)⁺, 97.21%.

### Example 6: Preparation of Compound 1.16

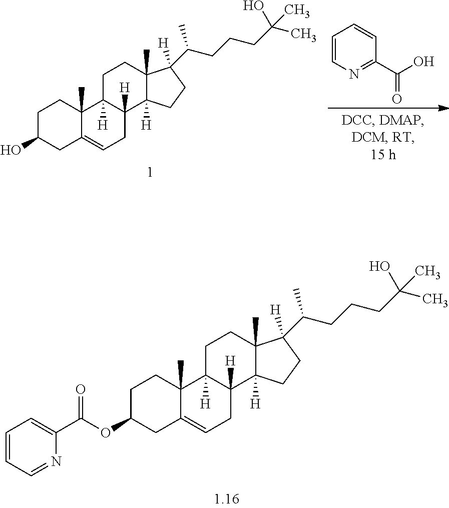

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl picolinate (1.16)

To a stirred solution of 25-hydroxy cholesterol 1 (80 mg, 0.198 mmol) in DCM (5 mL) was added picolinic acid (26 mg, 0.218 mmol), DMAP (12 mg, 0.099 mmol) and DCC (48 mg, 0.237 mmol). The resulting reaction mass was stirred at RT for 15 h. After completion of reaction (TLC monitoring), the reaction mixture was filtered through Celite® pad and washed with 70% EtOAc in hexane, the filtrate was collected and evaporated under reduced pressure to give crude residue, which was purified over prep-HPLC (method attached below) to get desired product 1.16 (50 mg, Yield: 49%) as off-white solid. ¹H-NMR (400 MHz, DMSO-d₆): δ 8.78 (d, J=4.4 Hz, 1H), 8.14 (d, J=7.6 Hz, 1H), 7.85 (t, J=8.0 Hz, 1H), 7.48 (m, 1H), 5.98 (s, 1H), 5 (s, 1H), 2.47-2.7 (m, 2H), 1.84-2.04 (m, 6H), 1.35-1.57 (m, 11H), 1.01-1.22 (m, 18H), 1.00-1.02 (m, 3H) and 0.69 (s, 3H). LCMS: 508.31 (M+H)⁺, 99.53%. LCMS: 508.31 (M+H)⁺, 99.53%.

### Example 7: Preparation of Compound 1.17

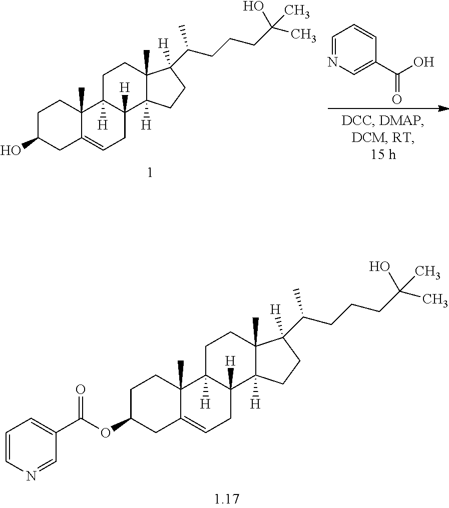

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl nicotinate (1.17)

To a stirred solution of 25-hydroxy cholesterol 1 (80 mg, 0.198 mmol) in DCM (5 mL) was added nicotinic acid (26 mg, 0.218 mmol), DMAP (12 mg, 0.099 mmol) and DCC (48 mg, 0.237 mmol). The resulting reaction mass was stirred at RT for 15 h. After completion of reaction (TLC monitoring), the reaction mixture was filtered through Celite® pad and washed with 50% EtOAc in hexane, the filtrate was collected and evaporated under reduced pressure to give crude residue, which was purified over prep-HPLC column chromatography (method attached below) to get desired product 1.17 (19 mg, Yield: 18%) as off-white solid. ¹H-NMR (400 MHz, DMSO-d₆): δ 9.24 (s, 1H), 8.78 (s, 1H), 8.35 (d, J=8.0 Hz, 1H), 7.43-7.46 (m, 1H), 5.42 (d, J=4.4 Hz, 1H), 4.86-4.94 (m, 1H), 2.46-2.48 (m, 2H), 1.90-2.00 (m, 4H), 1.76-1.82 (m, 3H), 1.56-1.64 (m, 2H), 1.34-1.48 (m, 9H), 1.20-1.29 (m, 10H), 1.09-1.14 (m, 7H), 0.92-0.99 (m, 3H) and 0.68 (s, 3H). LCMS: 508.25 (M+H)⁺, 98.34%.

### Example 8: Preparation of Compound 1.4B

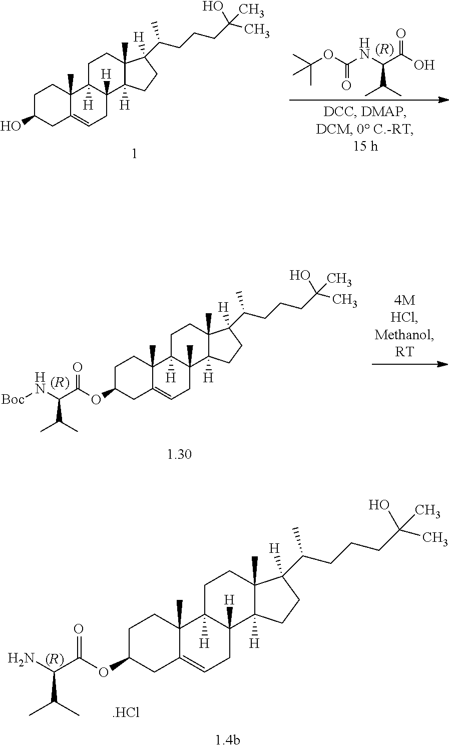

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-D-valinate (1.30)

To an ice-cold solution of (tert-butoxycarbonyl)-D-valine (108 mg, 0.498 mmol) in DCM (5 mL) was added DMAP (91 mg, 0.747 mmol), DCC (153 mg, 0.747 mmol) followed by addition of a solution of 25-hydroxy cholesterol 1 (100 mg, 0.249 mmol) in DCM (2.0 mL). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (20 mL), saturated solution of NaHCO₃ (20 mL) and water (20 mL) sequentially. The organic layer was dried over anhydrous Na₂SO₄, filtered and concentrated under reduced pressure to get crude. The crude was purified over flash column (Silica 12 g, redisep), eluted with 15% EtOAc in hexane to give pure compound 1.30 (120 mg, Yield: 80%). LCMS: 602.6 (M+H)⁺, 99.66%.

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl D-valinate hydrochloride salt (1.4b)

To an ice-cold solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-D-valinate 1.30 (120 mg, 0.214 mmol) in methanol (10 mL) was added 4M HCl in dioxane (6 mL) and resulting mixture stirred at RT for 2 h. After completion of reaction (TLC monitoring), the resulting reaction mixture was evaporated under reduced pressure to get crude residue, which was triturated with diethyl ether (20 mL×2 times) to get desired compound 1.4b (108 mg, Yield: Quantitative). ¹H-NMR (400 MHz, DMSO-d₆): δ 8.33 (s, 3H), 5.38 (s, 1H), 4.59-4.65 (m, 1H), 4.03 (br s, 1H), 3.86 (d, J=4.4 Hz, 1H), 2.33-2.39 (m, 3H), 2.15-2.17 (m, 2H), 1.78-1.91 (m, 6H), 1.19-1.63 (m, 20H), 1.14 (s, 6H), 1.04-1.09 (m, 6H) and 0.89-0.96 (m, 6H). LCMS: 502.4 (M+H)⁺, 98.11%.

### Example 9: Preparation of Compound 1.1

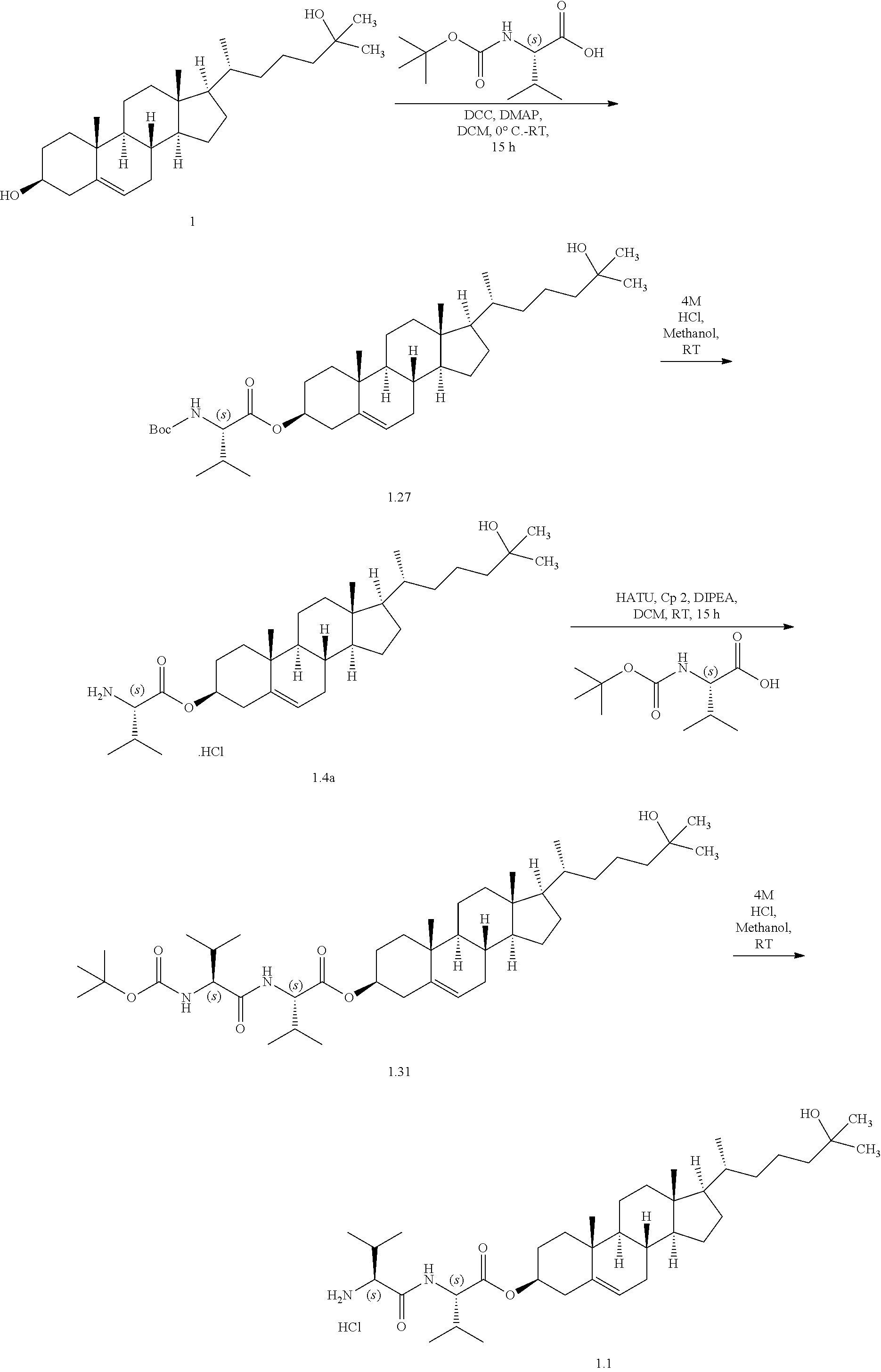

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-L-valinate (1.27)

To a stirred solution of (tert-butoxycarbonyl)-L-valine (323 mg, 1.49 mmol) in DCM (10 mL) was added DMAP (273 mg, 2.23 mmol), DCC (461 mg, 2.23 mmol) followed by addition of a solution of 25-hydroxy cholesterol 1 (300 mg, 0.746 mmol) in DCM (5.0 mL). The resulting reaction mass was stirred at RT for 15 h. After completion of reaction (TLC monitoring), the reaction mixture was filtered through Celite® pad and washed with 70% EtOAc in hexane. The filtrate was washed with 5% citric acid (20 mL), saturated solution of NaHCO₃ (20 mL) and water (20 mL) sequentially. The organic layer was dried over anhydrous Na₂SO₄, filtered and concentrated under reduced pressure to get crude. The crude was purified over flash column (Silica 12 g, Redisep), eluted with 25% EtOAc in hexane to give pure compound 1.27 (300 mg, Yield: 66%) as off white solid. ¹H-NMR (400 MHz, CDCl₃): δ 5.37 (s, 1H), 5.01-5.03 (m, 1H), 4.65-4.68 (m, 1H), 4.16-4.18 (m, 1H), 2.30-2.32 (m, 2H), 2.10-2.20 (m, 1H), 1.94-1.99 (m, 2H), 1.85-1.88 (m, 4H), 1.52-1.60 (m, 2H), 1.33-1.45 (m, 15H), 1.17-1.20 (m, 10H), 1.09-1.16 (m, 4H), 1.01-1.06 (m, 5H), 0.88-0.97 (m, 11H) and 0.67 (s, 3H). LCMS: 602.4 (M+H)⁺, 99.48%.

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl L-valinate hydrochloride (1.4a)

To an ice-cold solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-L-valinate 1.27 (300 mg, 0.498 mmol) in methanol (15 mL) was added 4M HCl in dioxane (8 mL) and resulting mixture stirred at RT for 2 h. After completion of reaction (TLC monitoring), the resulting reaction mixture was evaporated under reduced pressure to get crude residue, which was triturated with diethyl ether (10 mL×2 times) to get desired product 1.4a (250 mg, Yield: 93%) as off white solid. ¹H-NMR (400 MHz, DMSO-d₆): δ 8.35 (s, 3H), 5.38 (s, 1H), 4.61-4.64 (m, 1H), 4.02 (br s, 1H), 3.87 (d, J=5.4 Hz, 1H), 2.32-2.38 (m, 3H), 2.14-2.17 (m, 2H), 1.81-1.96 (m, 6H), 1.53-1.55 (m, 1H), 1.42-1.48 (m, 4H), 1.22-1.33 (m, 5H), 1.10-1.19 (m, 7H), 0.89-1.05 (m, 18H) and 0.65 (m, 3H).

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-L-valyl-L-valinate (1.31)

To a stirred solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl L-valinate hydrochloride 1.4a (120 mg, 0.223 mmol) in DCM (5 mL) added (tert-butoxycarbonyl)-L-valine (96 mg, 0.446 mmol), 1-[Bis(dimethylamino)methylene]-1H-1,2,3-triazolo[4,5-b]pyridinium-3-oxide hexafluorophosphate (HATU) (212 mg, 0.558 mmol) and DIPEA (194 μL, 1.11 mmol). The resulting reaction mass was stirred at RT for 15 h. After completion of reaction (TLC monitoring), the reaction mixture was diluted with sat. NH₄Cl (20 mL) and extracted with DCM (20 mL×2). The organic layer was dried over anhydrous Na₂SO₄, filtered and concentrated under reduced pressure to get crude. The crude was purified over flash column (Silica 12 g, Redisep), eluted with 25% EtOAc in hexane to give pure compound 1.31 (55 mg, Yield: 35%) as off white solid. ¹H-NMR (400 MHz, DMSO-d₆): δ 6.2 (s, 1H), 5.37 (s, 1H), 5.03-5.06 (m, 1H), 4.52-4.64 (m, 2H), 3.89-3.92 (m, 1H), 2.32 (d, J=8 Hz, 2H), 1.87-2.19 (m, 3H), 1.36-1.48 (m, 20H), 0.9-1.25 (m, 38H) and 0.67 (s, 3H). LCMS: 701 (M+H)⁺, 99.68%.

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl L-valyl-L-valinate hydrochloride (1.1)

To a stirred solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-L-valyl-L-valinate 1.31 (52 mg, 0.074 mmol) in methanol (5 mL) was added 4M HCl in dioxane (2 mL) and resulting mixture stirred at RT for 1 h. After completion of reaction (TLC monitoring), the resulting reaction mixture was evaporated under reduced pressure to get crude residue, which was triturated with acetonitrile (5 mL×2), diethyl ether (10 mL×2) and dried under high vacuum to get desired product 1.1 (40 mg, Yield: 85%) as off white solid. ¹H-NMR (400 MHz, DMSO-d₆): δ 8.54 (d, J=8 Hz, 1H), 8.11 (s, 3H), 5.35 (s, 1H), 4.5 (s, 1H), 4.17 (t, J=8 Hz, 1H), 3.72 (s, 1H), 2.24 (d, J=8 Hz, 2H), 1.7-2.1 (m, 8H), 0.89-1.57 (m, 44H) and 0.65 (s, 3H). LCMS: 601 (M+H)⁺, 94.52%.

### Example 10: Preparation of Compound 1.2

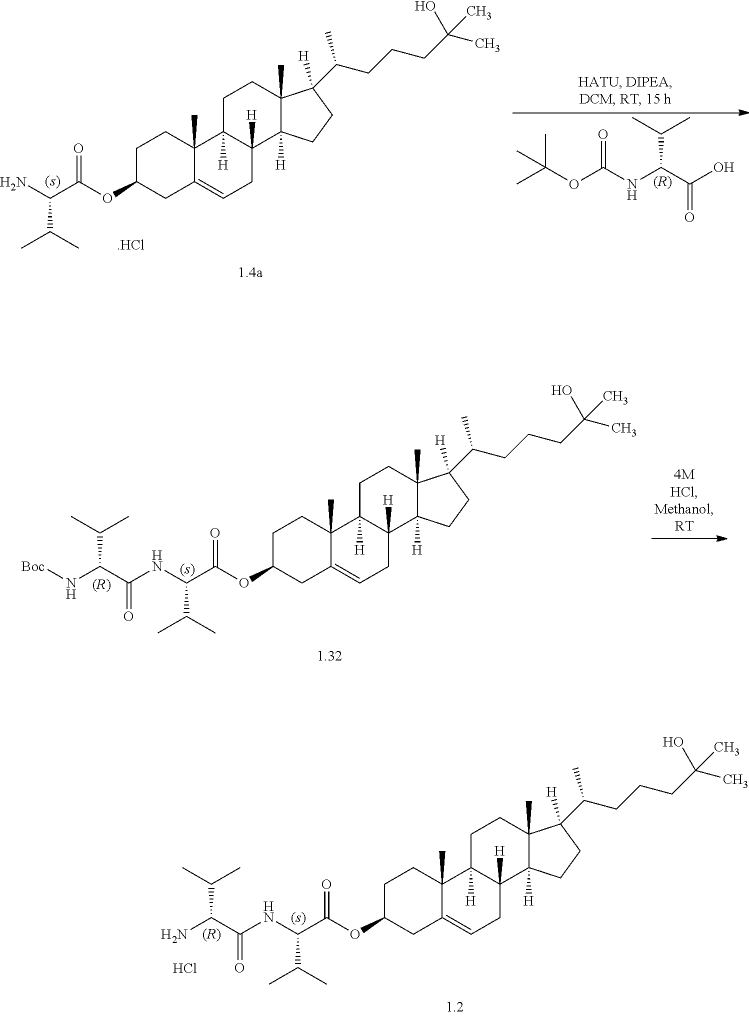

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-D-valyl-L-valinate (1.32)

To a stirred solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl L-valinate hydrochloride 1.4a (120 mg, 0.223 mmol) in DCM (5 mL) added (tert-butoxycarbonyl)-D-valine (96 mg, 0.446 mmol), HATU (212 mg, 0.558 mmol) and DIPEA (194 μL, 1.11 mmol). The resulting reaction mass was stirred at RT for 15 h. After completion of reaction (TLC monitoring), the reaction mixture was diluted with sat. NH₄Cl (20 mL) and extracted with DCM (20 mL×2). The organic layer was dried over anhydrous Na₂SO₄, filtered and concentrated under reduced pressure to get crude. The crude was purified over flash column (Silica 12 g, redisep), eluted with 20% EtOAc in hexane to give pure compound 1.32 (62 mg, Yield: 41%) as off white solid. ¹H-NMR (400 MHz, DMSO-d₆): δ 6.44 (d, J=8 Hz, 1H), 5.37 (s, 1H), 5.00 (bs, 1H), 4.53-4.44 (m, 2H), 3.99 (bs, 1H), 2.32 (d, J=8 Hz, 1H), 2.17-2.21 (m, 1H), 1.87-2.02 (m, 5H), 1.36-1.60 (m, 19H), 0.89-1.25 (m, 37H) and 0.67 (s, 3H). LCMS: 702 (M+H)⁺, 99.78%.

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl D-valyl-L-valinate hydrochloride (1.2)

To a stirred solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-D-valyl-L-valinate 1.32 (60 mg, 0.085 mmol) in methanol (5 mL) was added 4M HCl in dioxane (3 mL) and resulting mixture stirred at RT for 1 h. After completion of reaction (TLC monitoring), the resulting reaction mixture was evaporated under reduced pressure to get crude residue, which was triturated with acetonitrile (5 mL×2), diethyl ether (20 mL×2) and dried under high vacuum get desired product 1.2 (41 mg, Yield: 75%) as off white solid. ¹H-NMR (400 MHz, DMSO-d₆): δ 8.72 (d, J=8 Hz, 1H), 8.04 (s, 3H), 5.36 (s, 1H), 4.55 (s, 1H), 4.23 (t, J=8 Hz, 1H), 4.03 (s, 1H), 3.56 (d, J=8 Hz, 1H), 1.83-2.5 (m, 12H), 1.04-1.76 (m, 17H), 0.89-0.98 (24H) and 0.65 (s, 3H). LCMS: 602 (M+H)⁺, 99.54%.

### Example 11: Preparation of Compound 1.3a

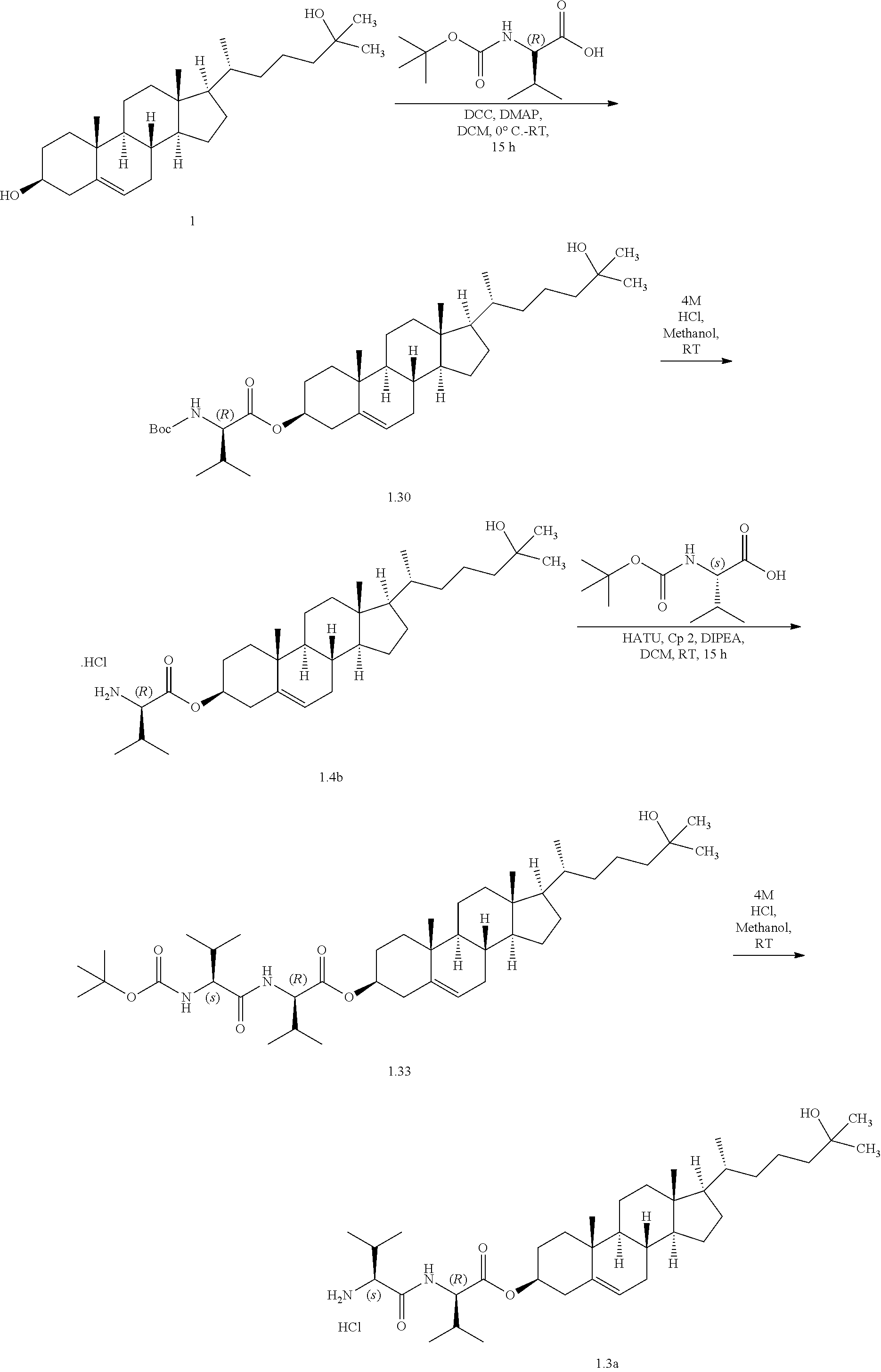

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-D-valinate (1.30)

To a stirred solution of (tert-butoxycarbonyl)-D-valine (323 mg, 1.49 mmol) in DCM (10 mL) was added DMAP (273 mg, 2.23 mmol), DCC (206 mg, 2.23 mmol) followed by addition of a solution of 25-hydroxy cholesterol 1 (300 mg, 0.746 mmol) in DCM (5 mL). The resulting reaction mass was stirred at RT for 15 h. After completion of reaction (TLC monitoring), the reaction mixture was filtered through Celite® pad and washed with 70% EtOAc in hexane. The filtrate was washed with 5% citric acid (20 mL), saturated solution of NaHCO₃ (20 mL) and water (20 mL) sequentially. The organic layer was dried over anhydrous Na₂SO₄, filtered and concentrated under reduced pressure to get crude. The crude was purified over flash column (Silica 12 g, Redisep), eluted with 20% EtOAc in hexane to give pure compound 1.30 (285 mg, Yield: 63%) as off white solid. ¹H-NMR (400 MHz, DMSO-d₆): δ 5.3 (s, 1H), 5.01-5.03 (m, 1H), 4.66 (bs, 1H), 4.18 (bs, 1H), 1.7-2.3 (m, 8H), 1.36-1.64 (m, 19H), 0.87-1.29 (m, 29H) and 0.67 (s, 3H). LCMS: 602 (M+H)⁺, 99.90%.

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl D-valinate hydrochloride (1.4b)

To an ice-cold solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-D-valinate 1.30 (280 mg, 0.465 mmol) in methanol (15 mL) was added 4M HCl in dioxane (15 mL) and resulting mixture stirred at RT for 2 h. After completion of reaction (TLC monitoring), the reaction mixture was evaporated under reduced pressure to get crude residue, which was triturated with diethyl ether (15 mL×2) to get desired product 1.4b (200 mg, Yield: 80%) as off white solid. ¹H-NMR (400 MHz, DMSO-d₆): δ 8.33 (s, 3H), 5.38 (s, 1H), 4.59-4.65 (m, 1H), 4.03 (br s, 1H), 3.86 (d, J=4.4 Hz, 1H), 2.33-2.39 (m, 3H), 2.15-2.17 (m, 2H), 1.78-1.91 (m, 6H), 1.19-1.63 (m, 20H), 1.14 (s, 6H), 1.04-1.09 (m, 6H) and 0.89-0.96 (m, 6H).

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-L-valyl-D-valinate (1.33)

To a stirred solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl D-valinate hydrochloride 1.4b (100 mg, 0.186 mmol) in DCM (10 mL) added (tert-butoxycarbonyl)-L-valine (80 mg, 0.372 mmol), HATU (176 mg, 0.465 mmol) and DIPEA (120 μL, 0.93 mmol). The resulting reaction mass was stirred at RT for 15 h. After completion of reaction (TLC monitoring), the reaction mixture was diluted with sat. NH₄Cl (20 mL) and extracted with DCM (20 mL×2). The combined organic layer was dried over anhydrous Na₂SO₄, filtered and concentrated under reduced pressure to get crude. The crude was purified over flash column (Silica 12 g, Redisep), eluted with 25% EtOAc in hexane to give desired compound 1.33 (55 mg, Yield: 42%) as off white solid. ¹H-NMR (400 MHz, DMSO-d₆): δ 639 (s, 1H), 5.38 (s, 1H), 5.10 (d, J=8 Hz, 1H), 4.66-4.67 (m, 1H), 4.49-4.52 (m, 1H), 3.88-3.92 (m, 1H), 2.18-2.31 (m, 2H), 2 1.84-2.19 (m, 8H), 1.34-1.57 (m, 21H), 0.90-1.27 (m, 32H) and 0.67 (s, 3H). LCMS: 701 (M+H)⁺, 99.68%.

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl L-valyl-D-valinate hydrochloride (1.3a)

To a stirred solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-L-valyl-D-valinate 1.33 (55 mg, 0.078 mmol) in methanol (5 mL) was added 4M HCl in dioxane (2 mL) and stirred at RT for 2 h. After completion of reaction (TLC monitoring), the resulting reaction mixture was evaporated under reduced pressure to get crude residue, which was triturated with acetonitrile (5 mL×2), diethyl ether (10 mL×2) and dried under high vacuum to get desired product 1.3a (41 mg, Yield: 80%) as off white solid. ¹H-NMR (400 MHz, DMSO-d₆): δ 8.56 (d, J=8 Hz, 1H), 8.1 (s, 3H), 5.35 (s, 1H), 4.5 (bs, 1H), 4.16 (t, J=8 Hz, 1H), 4.02 (s, 1H), 3.71 (s, 1H), 1.55-2.06 (m, 16H), 0.89-1.48 (m, 38H), 0.65 (s, 1H), LCMS: 601 (M+H)⁺, 93.14%.

### Example 12: Preparation of Compound 1.3b

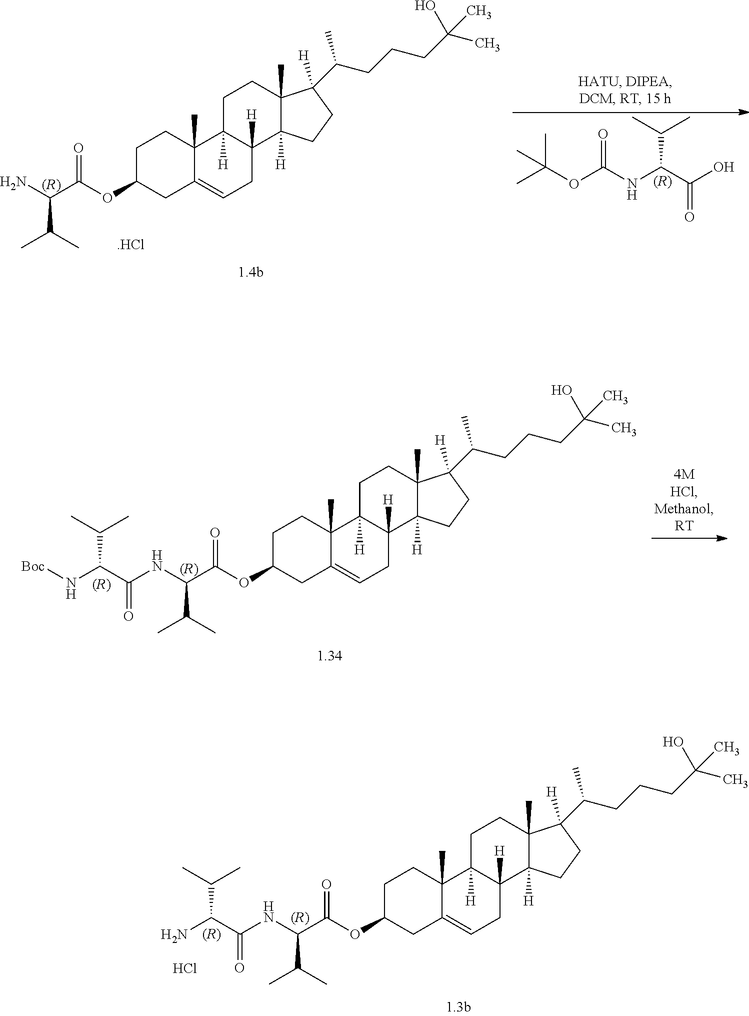

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-D-valyl-D-valinate (1.34)

To a stirred solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl D-valinate hydrochloride 1.4b (100 mg, 0.186 mmol) in DCM (10 mL) added (tert-butoxycarbonyl)-D-valine (80 mg, 0.372 mmol), HATU (176 mg, 0.465 mmol) and DIPEA (120 μL, 0.93 mmol). The resulting reaction mass was stirred at RT for 15 h. After completion of reaction (TLC monitoring), the reaction mixture was diluted with sat. NH₄Cl (20 mL) and extracted with DCM (20 mL×2). The combined organic layer was dried over anhydrous Na₂SO₄, filtered and concentrated under reduced pressure to get crude. The crude was purified over flash column (Silica 12 g, redisep), eluted with 25% EtOAc in hexane to give desired product 1.34 (65 mg, Yield: 50%) as off white solid. ¹H-NMR (400 MHz, DMSO-d₆): δ 639 (s, 1H), 5.38 (s, 1H), 5.10 (d, J=8 Hz, 1H), 4.66-4.67 (m, 1H), 4.49-4.52 (m, 1H), 3.88-3.92 (m, 1H), 2.18-2.31 (m, 2H), 2 1.84-2.19 (m, 8H), 1.34-1.57 (m, 21H), 0.90-1.27 (m, 32H) and 0.67 (s, 3H). LCMS: 701 (M+H)⁺, 99.78%.

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl D-valyl-D-valinate hydrochloride (1.3b)

To a stirred solution of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl (tert-butoxycarbonyl)-D-valyl-D-valinate 1.34 (65 mg, 0.092 mmol) in methanol (5 mL) was added 4M HCl in dioxane (3 mL) and stirred at RT for 1 h. After completion of reaction (TLC monitoring), the resulting reaction mixture was evaporated under reduced pressure to get crude residue, which was triturated with acetonitrile (10 mL×2), diethyl ether (10 mL×2) and dried under high vacuum to get desired product 1.3b (42 mg, Yield: 70%) as off white solid. ¹H-NMR (400 MHz, DMSO-d₆): δ 8.72 (d, J=8 Hz, 1H), 8.10 (s, 3H), 5.33 (s, 1H), 4.16 (t, J=8 Hz, 1H), 4.02 (s, 1H), 3.75 (s, 1H), 1.78-2.33 (m, 17H), 0.89-1.57 (m, 37H) and 0.68 (s, 3H). LCMS: 602 (M+H)⁺, (ELSD) 97.90%, (UV) 92.89%.

### Example 13: Preparation of Compound 1.19

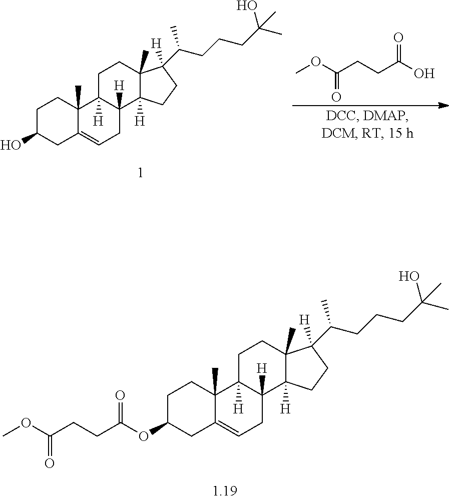

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl methyl succinate (1.19)

To a stirred solution of 4-methoxy-4-oxobutanoic acid (65 mg, 0.496 mmol) in DCM (5 mL) was added DMAP (90 mg, 0.744 mmol), DCC (153 mg, 0.744 mmol) followed by addition of a solution of 25-hydroxy cholesterol 1 (100 mg, 0.248 mmol) in DCM (2 mL). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (10 mL), saturated solution of NaHCO₃ (10 mL) and water (10 mL) sequentially. The organic layer was dried over anhydrous Na₂SO₄, filtered and concentrated under reduced pressure to get crude. The crude was purified over flash column (Silica 12 g, Redisep), eluted with a gradient of 0-30% EtOAc in hexane and run time of 40 min to get desired product 1.19 (30 mg, Yield: 23%) as off white solid. ¹H-NMR (400 MHz, DMSO-d₆): δ 5.37 (s, 1H), 4.62 (t, J=8 Hz, 1H), 3.69 (s, 3H), 2.61 (s, 4H), 2.32 (d, J=8 Hz, 2H), 1.84-2.02 (m, 5H) and 1.04-1.53 (m, 36H). LCMS: 534 (M+H+17)⁺, 99.01%.

### Example 14: Preparation of Compound 1.8

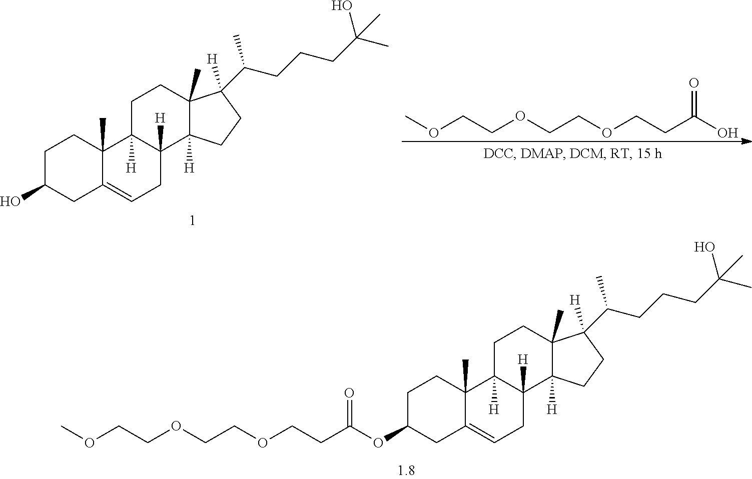

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl 3-(2-(2-methoxyethoxy)ethoxy)propanoate (1.8)

To a stirred solution of 3-(2-(2-methoxyethoxy)ethoxy)propanoic acid (71 mg, 0.372 mmol) in DCM (5 mL) was added DMAP (60 mg, 0.496 mmol), DCC (102 mg, 0.496 mmol) followed by addition of a solution of 25-hydroxy cholesterol 1 (100 mg, 0.248 mmol) in DCM (2 mL). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (10 mL) and brine (25 mL) sequentially. The organic layer was dried over anhydrous Na₂SO₄ filtered and concentrated under reduced pressure to get greasy yellow residue. This yellow residue was diluted with diethyl ether (20 mL) filtered over Celite® pad and washed with diethyl ether (10 mL×2). The filtrate was evaporated and purified over flash column (Silica 12 g, Redisep), eluted with a gradient of 0-80% Ethyl acetate in hexane with a run time of 30 min to give desired product 1.8 (85 mg, Yield: 59%) as off white solid. ¹H-NMR (400 MHz, DMSO-d₆): δ 5.7 (s, 1H), 4.62-4.63 (m, 1H), 3.76 (t, J=8 Hz, 2H), 3.65 (s, 6H), 3.53-3.54 (m, 2H), 3.37 (s, 3H), 2.58 (t, J=8 Hz, 2H), 2.30 (d, J=8 Hz, 2H), 1.39-1.98 (m, 24H), 0.92-1.34 (m, 14H) and 0.67 (s, 3H). LCMS: 594.6 (M+H+17)⁺, 99.82%.

### Example 15: Preparation of Compound 1.9

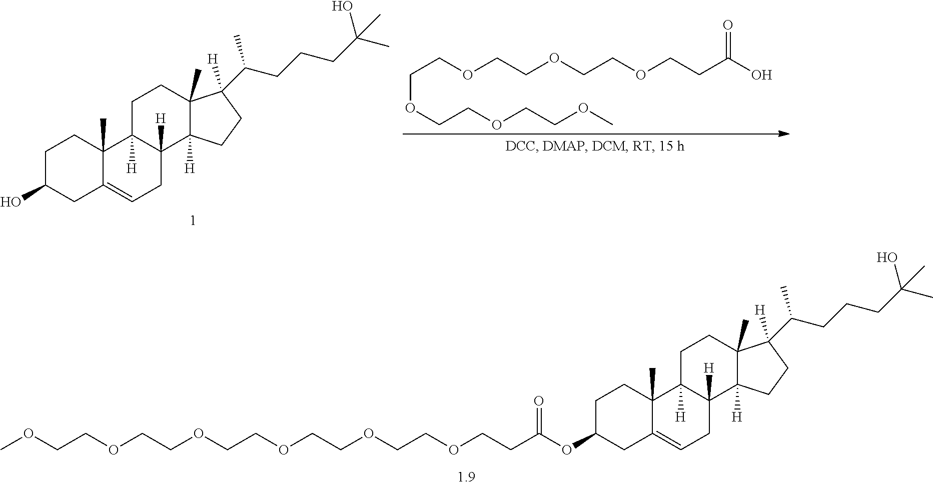

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl 2,5,8,11,14,17-hexaoxaicosan-20-oate (1.9)

To a stirred solution of 2,5,8,11,14,17-hexaoxaicosan-20-oic acid (104 mg, 0.322 mmol) in DCM (5 mL) was added DMAP (60 mg, 0.496 mmol), DCC (102 mg, 0.496 mmol) followed by addition of a solution of 25-hydroxy cholesterol 1 (100 mg, 0.248 mmol) in DCM (2 mL). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (20 mL) and brine (10 mL) sequentially. The organic layer was dried over anhydrous Na₂SO₄, filtered and concentrated under reduced pressure to get greasy yellow residue. This yellow residue was diluted with diethyl ether (20 mL), filtered over Celite® pad and washed with diethyl ether (10 mL×2). The filtrate was evaporated and purified over flash column (Silica 12 g, Redisep), eluted with a gradient of 0-80% EtOAc in hexane with a run time 30 min to get desired product 1.9 (105 mg, Yield: 59%) as off white solid. ¹H-NMR (400 MHz, DMSO-d₆): δ 5.37 (s, 1H), 4.61-4.63 (m, 1H), 3.53-3.7 (m, 25H), 2.5 (t, J=8 Hz, 2H), 2.32 (d, J=2 Hz, 2H), 1.84-1.2.02 (m, 5H), 1.01-1.55 (m, 33H) and 0.68 (s, 3H). LCMS: 726 (M+H+17)⁺, 99.57%.

### Example 16: Preparation of Compound 1.10

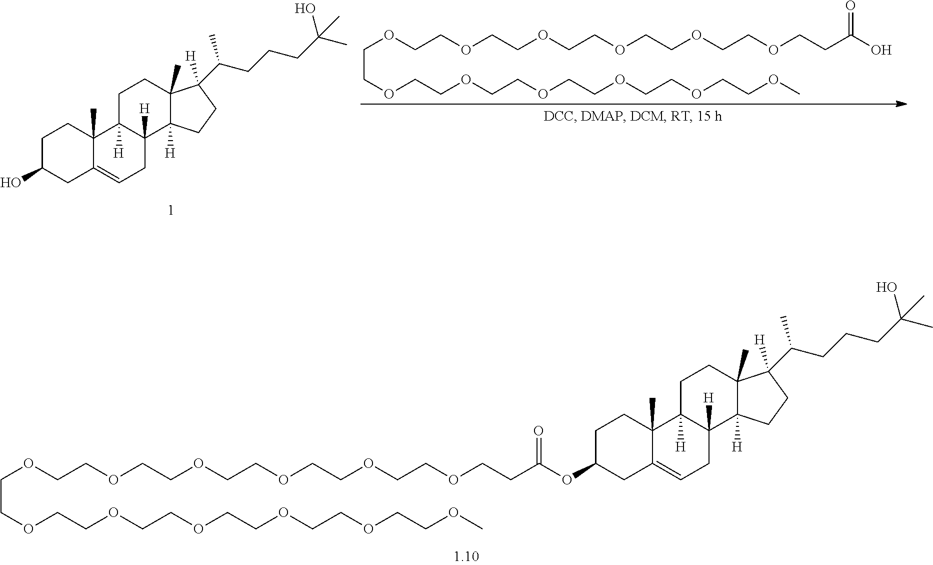

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl 2,5,8,11,14,17,20,23,26,29,32,35-dodecaoxaoctatriacontan-38-oate (1.10)

To a stirred solution of 2,5,8,11,14,17,20,23,26,29,32,35-dodecaoxaoctatriacontan-38-oic acid (145 mg, 0.248 mmol) in DCM (5 mL) was added DMAP (60 mg, 0.496 mmol), DCC (102 mg, 0.496 mmol) followed by addition of a solution of 25-hydroxy cholesterol 1 (100 mg, 0.248 mmol) in DCM (2 mL). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (20 mL) and brine (10 mL) sequentially. The organic layer was dried over anhydrous Na₂SO₄, filtered and concentrated under reduced pressure to greasy yellow residue. This yellow residue was diluted with diethyl ether (20 mL), filtered over Celite® pad and washed with diethyl ether (10 mL×2). The filtrate was evaporated and purified over flash column (Silica 12 g, Redisep), eluted with a gradient of 0-10% MeOH in DCM with a run time 30 min to give desired compound 1.10 (105 mg, Yield: 43%) as off white solid. ¹H-NMR (400 MHz, DMSO-d₆): δ 5.37 9 s, 1H), 4.61 (bs, 1H), 3.37-3.75 (m, 50H), 2.58 (t, J=8 Hz, 2H), 2.30 (d, J=8 Hz, 2H), 1.49-1.82 (m, 5H), 0.87-1.58 (m, 32H) and 0.67 (s, 3H). LCMS: 990 (M+H+17)⁺, 99.34%.

### Example 17: Preparation of Compound 1.11

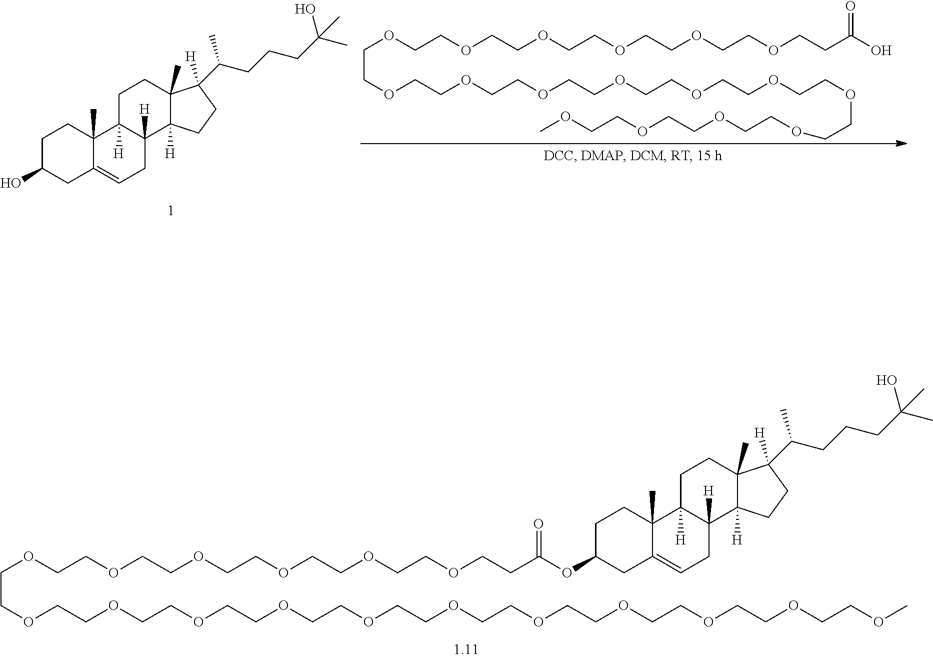

### Preparation of (3S,8S,9S,10R,13R,14S,17R)-17-((R)-6-hydroxy-6-methylheptan-2-yl)-10,13-dimethyl-2,3,4,7,8,9,10,11,12,13,14,15,16,17-tetradecahydro-1H-cyclopenta[a]phenanthren-3-yl 2,5,8,11,14,17,20,23,26,29,32,35,38,41,44,47,50-heptadecaoxatripentacontan-53-oate (1.11)

To a stirred solution of 2,5,8,11,14,17,20,23,26,29,32,35,38,41,44,47,50-heptadecaoxatripentacontan-53-oic acid (240 mg, 0.297 mmol) in DCM (5 mL) was added DMAP (60 mg, 0.496 mmol), DCC (102 mg, 0.496 mmol) followed by addition of a solution of 25-hydroxy cholesterol 1 (100 mg, 0.248 mmol) in DCM (2 mL). The resulting reaction mass was stirred at RT for 15 h. After completion of reaction (TLC monitoring), the reaction mixture was filtered through Celite® pad and washed with 50% EtOAc in hexane. The filtrate was washed with 5% citric acid (25 mL) and brine (25 mL) sequentially. The organic layer was dried over anhydrous Na₂SO₄, filtered and concentrated under reduced pressure to get greasy yellow residue. This yellow residue was diluted with diethyl ether (20 mL) filtered over Celite® pad and washed with diethyl ether (10 mL×2). The filtrate was evaporated and purified over flash column (Silica 12 g, Redisep), eluted with a gradient of 0-10% MeOH in DCM with a run time of 30 min. The compound so obtained after purification was further triturated with chilled diethyl ether (20 mL×2) and dried under reduced pressure to get desired product 1.11 (45 mg, Yield: 15%) as off white solid. ¹H-NMR (400 MHz, DMSO-d₆): δ 5.37 (s, 1H), 4.61 (m, 1H), 3.3-3.7 (m, 71H), 2.58 (t, J=8 Hz, 2H), 1.99 (d, J=8 Hz, 2H), 1.61-1.86 (m, 5H) and 0.92-1.48 (m, 34H). LCMS: 1212 (M+H+17)⁺, 99.48%.

### Example 18: Differential Scanning Fluorimetry (DSF)

2.4 μL of 10 mM compound stocks in DMSO were added to the wells of a 96-well plate containing 51.6 μL of 7 μM recombinant alpha-crystallin core domain (ACD) of cryAB protein (residues 68-153) in DSF buffer (50 mM Tris-HCl, 100 mM NaCl, 0.5 mM ZnCl₂, 0.5 mM MgCl₂, 0.5 mM CaCl₂, pH8.0), and subsequently 6 μL 10× SYPRO orange protein gel stain (Invitrogen) was added for a final volume of 60 μL per well. Each well was mixed, and then 10 μL was added in quadruplicates to a MicroAmp Optical 384-well reaction plate with barcode (ThermoFisher). The plate was sealed with a MicroAmp Optical adhesive film (ThermoFisher), centrifuged at 1,000×g for 2 min, and loaded into a QuantStudio 6 Flex real-time PCR system (Applied Biosystems). The DSF assay was performed by heating the plate from 30° C. to 80° C. with 0.5° C. increments per cycle over 100 cycles. Each cycle was held for 90 s after reaching the temperature, and the fluorescent signal (520 nm excitation/558 nm emission) was measured.

The data curve was fitted using a Boltzmann model (GraphPad Prism Version 7) for a fitting range from 25° C. to the temperature at which the fluorescence signal was highest plus 3-5° C. The Tₘ was determined to be the temperature at the midpoint between the highest and lowest fluorescent signals on the curve. Results of the assay for compounds 1.18, 1.21 and 1.25 are in the following table and FIG. 1.

| | | Δ Tm (° C.) |
|---|---|---|
| Compound | Structure | Mean (n = 2) |
| 1.18 | 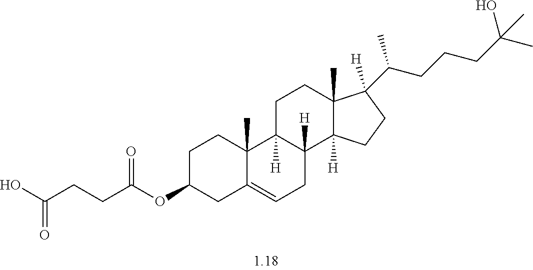 | −5.7 |
| 1.21 | 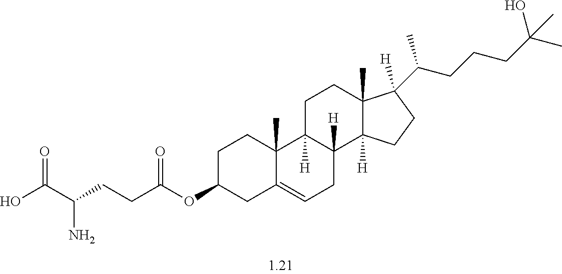 | −1.7 |
| 1.25 | 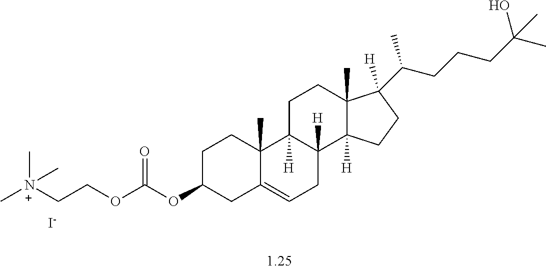 | −1.8 |

### Example 19: Microscale Thermophoresis (MST) for Direct Binding to CRYAB (R120G)

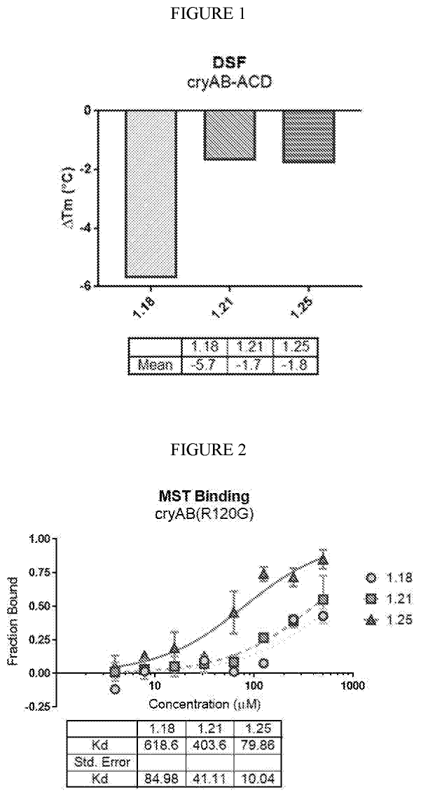

MicroScale Thermophoresis was performed on mixed oligomers of full length cryAB(R120G) combined with fluorescently-labelled cryAB(E87C)-ACD. The ACD point mutant construct was labelled with a thiol reactive dye (AlexaFluor™-488 C5 Maleimide, Molecular Probes). 100 nM of AlexaFluor-488 labeled cryAB(E87C)-ACD was combined with 10 μM full length cryAB(R120G) and incubated at room temperature for at least 15 minutes. Compounds were diluted to 10 mM in DMSO then serially diluted 2-fold in DMSO. To 9 μL of MST-TP buffer (50 mM Tris, 150 mM NaCl, 10 mM MgCl2, 0.05% Tween-20, 0.1% PEG-8000, pH7.4) was added 1 μL of compound in DMSO followed by 10 μL cryAB solution for a final concentration of 5 μM cryAB in 5% DMSO. Samples were incubated for 15 minutes at room temperature, then applied to Monolith NT. 115 Capillaries (MO-K022). Capillaries were loaded into a Monolith NT. 115 (NanoTemper Technologies GmbH) then analyzed (80% Blue laser power, Medium MST power, standard conditions). Response to ligand titration was determined at the 1.5 s threshold and fit to the K_{D} model using NanoTemper analysis software. Fraction bound was normalized to the curve fit. Results of the assay for compounds 1.18, 1.21 and 1.25 are in FIG. 2.

### Example 20: Amyloid Disaggregation Assay

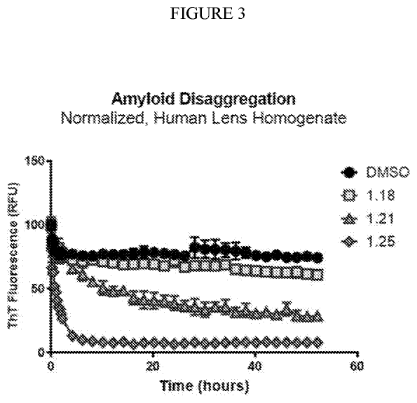

The procedure used to evaluate disassembly of amyloids in ex vivo cataractous solutions was adapted from Chemerovski-Glikman et al., Scientific Reports 8, Article number: 9341 (2018). Nuclear and cortical lens tissue from human donors (92F, 96M) was homogenized in PBS with protease inhibitor for 60 seconds on ice. The protein concentration of the lysate was measured by Bicinchoninic Acid Assay (Pierce BCA Protein Assay Kit, ThermoFisher), and then the lysate was diluted to a total protein concentration of 0.075 mg/mL. Thioflavin T dye (Sigma-Aldrich) was added to a final concentration of 1 μM. After an incubation step, compound was added to a final concentration of 10 μM (20 μM for 1.18 due to limited solubility) in 5% DMSO. After incubation with orbital shaking at 37° C., the fluorescence was read every two hours for 94 hours at an excitation wavelength of 440 nm and emission wavelength of 485 nm. Results of the assay for compounds 1.18, 1.21, 1.25 or control are in FIG. 3.

### Example 21: Chemical and Plasma Stability

10 mM compound working solutions were made by adding 10 μL of a 50 mM DMSO stock solution in 40 μL methanol. To make 20 μM compound working solutions, 1 μL of a 10 mM stock solution is added to 499 μL of methanol. Internal standard and quenching solutions were prepared by dissolving 1 mg/mL of terfenadine or tolbutamide in appropriate amounts of DMSO. A quenching solution was prepared containing 25 ng/ml terfenadine plus 50 ng/ml tolbutamide by diluting both stock solutions together in a 1:1 mixture of methanol:acetonitrile. Phosphate buffered saline (PBS) solutions at pH5 and pH7 were prepared by adjusting the pH of stock PBS with concentrated hydrochloric acid.

To tubes labeled with a pre-determined time point (0, 5, 15, 30, 60, 120 min) was added 2 μL of 20 μM compound working solution. In each tube, 38 μL of PBS buffer was added to the vial, vortexed, and incubated at 37° C. for the corresponding time. After incubation, 300 μL of the quenching solution containing internal standard was added to the tube and vortexed to mix. The sample was centrifuged at 4000 rpm for 15 min, and 100 μL of supernatant transferred to a 0.65 ml tube for LC/MS analysis. Each compound and timepoint was analyzed by reverse phase HPLC with MS detection performed using a Sciex API 4500 Qtrap. Results of the chemical stability assay are in the following table.

| | Chem stability | Chem stability |
|---|---|---|
| Compound | (t ½ @ pH = 5) | (t ½ @ pH = 7) |
| 1.18 | >500 min | >500 min |
| 1.25 | >500 min | >500 min |

A 4 mM compound stock solution was prepared by dissolving the appropriate amount of compound in MeOH, then a 10 μM compound working solution was prepared by adding 2 μL aforementioned 4 mM stock solution in 78 μL DMSO. A 10 mM control stock solution was prepared by dissolving the appropriate amount of tetracaine in DMSO, then a 10 μM control working solution was prepared by diluting the stock in DMSO. A 1 mg/ml terfenadine mixed with 2 mg/mL tolbutamide stock solution was prepared by dissolving an appropriate amount of each in DMSO, then a 1 ng/mL terfenadine mixed with 2 ng/mL tolbutamide internal standard solution was prepared by diluting the stock solution in methanol:acetonitrile (1:1, v/v). Both stock and internal standard solutions were stored at 4° C.

Whole blood was centrifuged (obtained from in life group or donors) at 4000 rpm at 4° C. for 15 min to obtain plasma. Plasma was stored at −80° C. for future use. Before use, plasma was pre-warmed at 37° C. in a water bath for 15 min (500 μL per compound or control). After warming, 5 μL compound or control was added into 495 μL plasma, then vortexed to mix. At each time point (0, 5, 15, 30, 60, and 120 min), 30 uL reaction mixture was aliquoted, then 300 L internal standard was added and vortexed to stop the reaction. Samples were spun at 4000 rpm for 15 min at 4° C., then 100 μL of the supernatant was removed for LC-MS/MS analysis. The MS detection was performed using a Sciex API 4000 Qtrap and 6500 Plus instrument, and each compound was analyzed by reverse phase HPLC. Results of the plasma stability assay are in the following table.

| | Plasma stability | Plasma stability |
|---|---|---|
| Compound | (rabbit) t ½ | (human) t ½ |
| 1.18 | >500 min | >500 min |
| 1.25 | >500 min | >500 min |

### Example 22: Solubility Assay

A compound described herein and 25-hydroxycholesterol are weighed into Eppendorf tubes and 0.5 mL of water is added for injection. The Eppendorf tubes are incubated at 37° C. for 6 h shaking at 200 rpm. The Eppendorf tubes are centrifuged for 10 min at 13,500 rpm, then 250 L of liquid is pipetted out and is filtered through a 0.45 m disposable syringe filter. The filtrates are collected and are diluted further with water for injection. The absorbance of the filtrates is measured by UV spectroscopy. The solubilities of the compound and 25-hydroxycholesterol are calculated in mg/mL. The compounds or salts described herein may demonstrate 10% or more increased aqueous solubility relative to 25-hydroxycholesterol.

### Example 23: Cellular Uptake Assay

A compound described herein and 25-hydroxycholesterol are incubated with cultured primary rabbit corneal cells in DPBS for 10 minutes. The cells are then washed three times with ice-cold HEPES buffer. The cells are lysed overnight with 1 mL 0.05% (w/v) Triton X-100 in 1 N NaOH at room temperature and then 500 μL aliquots from each well are transferred to scintillation vials containing a 5 mL scintillation cocktail. The samples are subjected to liquid scintillation spectrophotometry using a scintillation counter. The rate of uptake to protein content is then normalized for each well. Then, the amount of protein in the cell lysate is measured using bovine serum albumin as the standard. The measurements of the amount of protein in the cell lysate are then compared. The compound may demonstrate 10% or more increased cellular uptake than 25-hydroxycholesterol.

### Example 24: Lens, Corneal and Retinal Exposure

Formulations comprising a compound described herein are tested for lens, corneal and retinal exposure. New Zealand white albino rabbits are given six doses over three days for each topical arm, and a single injection for intravitreal and intracameral injections, with time points at two hours and 24 hours post-injection. The rabbits are scored to determine the safety of the compound. Rabbits are observed on day 0 (the day of dosing), day 1, day 2 and day 3.

The exposure of a compound described herein is measured in the cornea of the rabbits. To get to the cornea, the compounds or salts described herein must pass through the cornea, or sclera, to access the internal structures of the eye.

The exposure of the compounds or salts described herein is measured in the retina of the rabbits. This can demonstrate fast diffusion because the retina is a lipid rich, soft tissue area in the back of the eye.

The exposure of the compound is measured in the ciliary bodies of the rabbits. This can demonstrate fast diffusion because the ciliary body is a lipid-rich soft tissue in the anterior of the eye.

### Example 25: Ocular Distribution after Intravitreal Administration

### Animals

New Zealand White rabbits from the Western Oregon Rabbit Company are used for this study. 12 animals are used and all animals used are male. Prior to treatment initiation, selection of animals for the study is based on a visual appraisal of good clinical condition and body weight specifications. Animals selected for use in this study are as uniform in age and weight as possible. Animal's weights range from about 2.58 to about 3.22 kilograms at the start of the experiment. All animals are healthy at the time of animal selection. All animals are identified by ear tag and by cage cards listing the animal identification number, study number, group, and sex of the animal.

The animals are housed in individual cages within the same room during the study. Primary enclosures are as specified in the USDA Animal Welfare Act (9 CFR, Parts 1, 2, and 3) and as described in the Guide for Care and Use of Laboratory Animals (National Research Council of the Academies, 2011, National Academy Press).

No other species are housed in the same room. The room is well ventilated (greater than 10 air changes per hour) with at least 60% fresh air. A 12-hour light/12-hour dark photoperiod is maintained, except when rooms are illuminated during the dark cycle to accommodate necessary study procedures. Room temperatures are maintained as per ASI SOPs.

Animals have ad libitum access to species specific chow. No contaminants are known to be present in the diet at levels that will interfere with the results of this study. Chlorinated, municipal tap water is made available ad libitum to each animal via water bottles. No contaminants are known to be present in the water at levels that would interfere with the results of this study. Records of annual water quality testing are maintained in the ASI archives. All study animals are acclimated to their designated housing for 8 to 16 days prior to the first day of dosing.

Prior to placement on study, each animal undergoes an ophthalmic examination (slit-lamp biomicroscopy and indirect ophthalmoscopy). Ocular findings are scored according to a modified McDonald-Shadduck Scoring System and are recorded on a standardized data sheet. The acceptance criteria for placement on study are scores of “0” for all variables. Animals are assigned to one of two experimental groups based on body weight.

### Formulations

A compound described herein is tested at different concentrations, 3% weight/volume (formulation 1) and 0.5% weight/volume (formulation 2). The formulations are refrigerated at 4° C. prior to use. Prior to administration, the formulations are warmed to room temperature. There are no noted color changes or signs of microbial growth.

### Dosing

Animals are assigned to one of two experimental groups based on body weight, such that each group contains 6 animals. Animals in group 1 are administered 25 μL for formulation 1 by intravitreous injection, while animals in group 2 are administered 25 μL of formulation 2. Within each group animals are further divided into 3 time points, 2 hours, 1 day and 7 days. Each time point consists of two animals. Administration of formulations 1 and 2 occurs at time 0.

For intravitreal injections, animals are anesthetized with an intramuscular injection of ketamine hydrochloride (12 to 20 mg/kg) and xylazine (5 mg/kg). One to two drops of topical proparacaine hydrochloride anesthetic (0.5%) are applied to the animal's eyes prior to the surgical procedure. The eyes are cleaned with Betadine and then rinsed with balanced salt solution (BSS). Test article is drawn up directly into a 0.3 mL insulin syringe with a 31G 5/16 inch needle, and injections are made 4 to 5 mm away from the limbus. Once the needle is inserted, 25 μL of the test article is injected. The needle is removed and the eye is rinsed with BSS. Triple antibiotic ophthalmic ointment is administered in both eyes of each animal following the injection procedure. Animals are monitored during recovery.

General health observations are recorded daily starting on Day 0 and continue throughout the course of the study. Gross ocular examinations, which consisted of a visual appraisal of swelling, discharge, and irritation to the eye, are taken daily starting on Day 0 and continue throughout the course of the study.

### Sample Collection

Animals are euthanized at the designated time points by an intravenous injection of pentobarbital (150 mg/kg). The euthanasia procedure is performed in compliance with the 2013 American Veterinary Medical Association (AVMA) Guidelines on Euthanasia. Immediately following euthanasia, both eyes from each animal are enucleated, dissected, and the ocular tissues were collected. Samples from each eye in each animal remain separate and are not pooled. Samples are flash frozen in liquid nitrogen and stored at −60 to −80° C. until LC-MS/MS analysis. The concentration of compound or metabolite thereof in each collected sample is measured by LC-MS/MS. Standards are prepared in blank homogenized New Zealand white rabbit ocular tissues, vitreous humor, or plasma. Working solutions are prepared in 50:50 acetonitrile:water. Working solutions are then added to the appropriate matrix to make calibration standards. Standards are treated identically to the study samples. Tissue and humor samples are manually extracted via precipitation with acetonitrile.

### HPLC Conditions

Instrument: Waters Acquity UPLC; Column: Waters BEH phenyl, 30×2.1 mm id, 1.7 μm; Aqueous Reservoir (A):0.1% formic acid in water; Organic Reservoir 0.1% formic acid in acetonitrile; Gradient Program:

| Time (min) | Grad. Curve | % A | % B |
|---|---|---|---|
| 0.0 | 6 | 60 | 40 |
| 2.25 | 6 | 0 | 100 |
| 2.4 | 6 | 60 | 40 |
| 3 | 6 | 60 | 40 |

HPLC Flow rate: 800 μL/min; Injection volume: 10 μL; Column temperature: 40° C.; Sample temperature: 8° C.; Strong autosampler wash: 1:1:1 (v:v:v) water:methanol:isopropanol with 0.2% formic acid; Weak autosampler wash:4 mM ammonium formate

### Mass Spectrometer Conditions

Instrument: Waters Xevo TQ-S; Interface: Electrospray; Mode: Multiple reactions monitoring; Nebulizer gas: 7 bar; Desolvation gas: 1000 L/hr; Cone gas: 150 L/hr; Collision gas: 0.15 mL/min; Desolvation temp: 450° C.; Capillary voltage: 3 kV

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula (I): 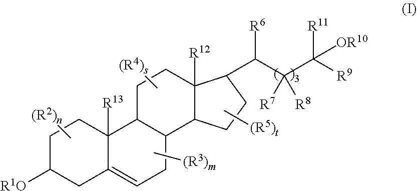
or a salt thereof, wherein:
R¹ and R¹⁰ are independently selected from hydrogen, —C(O)(CR²⁰₂)₁₋₆NR²¹₂, —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)₂, wherein at least one of R¹ and R¹⁰ is not hydrogen;
R², R³, R⁴, and R⁵ are independently selected at each occurrence from halogen, —OR¹⁵, —SR¹⁵, —OSO₃R¹⁵, —OPO₃R¹⁵, —N(R¹⁵)₂, —C(O)R¹⁵, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN;
R⁷ and R⁸ are independently selected at each occurrence from hydrogen, halogen, —OR¹⁵, —SR¹⁵, —OSO₃R¹⁵, —OPO₃R¹⁵, —N(R¹⁵)₂, —C(O)R¹⁵, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN;
n, s, m, and t are independently selected from 0, 1, 2, 3, 4, or 5;
R⁶, R⁹, R¹¹, R¹², and R¹³ are independently selected at each occurrence from C₁-C₆ alkyl optionally substituted with one or more substituents independently selected from halogen, —OR¹⁵, —SR¹⁵, —N(R¹⁵)₂, —NO₂, and —CN;
R¹⁵ is independently selected at each occurrence from hydrogen, and C₁-C₆ alkyl optionally substituted with one or more substituents independently selected from halogen, —CN, —NO₂, —NH₂, —OH, and —OCH₃;
R²⁰ is independently selected at each occurrence from hydrogen, halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;
R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —SR²², —S—S—R²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —S—C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;
or two R²¹ groups are taken together with the atoms to which they are attached form a heterocycle, optionally substituted with one or more R²⁴;
R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;
R²³ is selected from:
—C₁₋₆alkylene-(X¹—C₁₋₆alkylene)₁₋₂₄-X¹—C₁₋₆alkyl, wherein X¹ is independently selected at each occurrence from O, S, and NH and wherein R²³ is optionally substituted on a carbon or nitrogen atom with one or more substituents independently selected from R²⁴; and
a 3- to 12-membered heterocycle optionally substituted with one or more substituents independently selected from R²⁴;
R²⁴ is independently selected at each occurrence from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and
X² is independently selected at each occurrence from O and NH.

2. The compound or salt of claim 1, wherein R¹ is selected from —C(O)(CR²⁰₂)₁₋₆NR²¹₂, —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)₂.

3. The compound or salt of claim 2, wherein R¹⁰ is hydrogen.

4. The compound or salt of claim 2, wherein R¹ and R¹⁰ are independently selected from hydrogen and —C(O)(CR²⁰₂)₁₋₆NR²¹₂.

5. The compound or salt of claim 4, wherein —C(O)(CR²⁰₂)₁₋₆NR²¹₂ is selected from —C(O)CR²⁰₂NR²¹₂, —C(O)(CR²⁰₂)₂NR²¹₂, —C(O)(CR²⁰₂)₃NR²¹₂, —C(O)(CR²⁰₂)₄NR²¹₂, —C(O)(CR²⁰₂)₅NR²¹₂, and —C(O)(CR²⁰₂)₆NR²¹₂.

6. The compound or salt of claim 4, wherein R¹ and R¹⁰ are independently selected from hydrogen and —C(O)CR²⁰₂NR²¹₂.

7. The compound or salt of claim 6, wherein —C(O)CR²⁰₂NR²¹₂ is selected from:

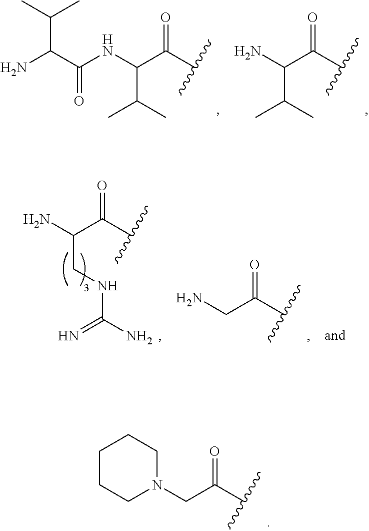

8. The compound or salt of claim 7, wherein —C(O)CR²⁰₂NR²¹₂ is selected from:

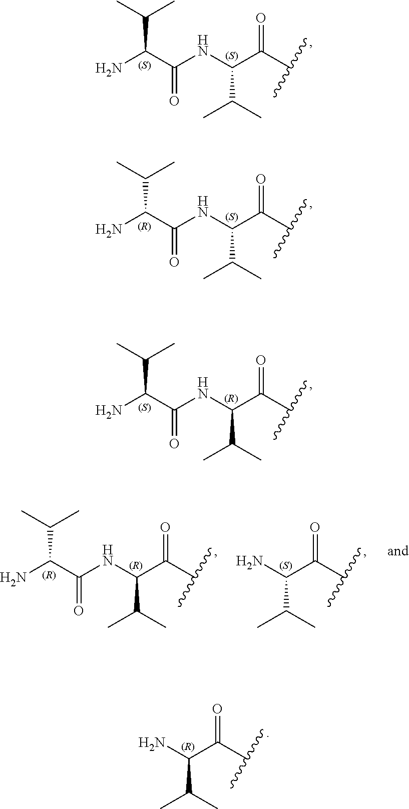

9. The compound or salt of claim 4, wherein R¹ and R¹⁰ are independently selected from hydrogen and —C(O)(CR²⁰₂)₅NR²¹₂.

10. The compound or salt of claim 9, wherein R¹ and R¹⁰ are independently selected from hydrogen and

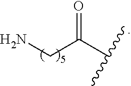

11. The compound or salt of claim 2, wherein R¹ and R¹⁰ are independently selected from hydrogen and —C(O)R²³.

12. The compound or salt of claim 11, wherein R²³ is selected from —C₁₋₆alkylene-(X¹—C₁₋₆ alkylene)₁₋₂₄-X¹—C₁₋₆alkyl.

13. The compound or salt of claim 12, wherein R²³ is selected from —C₁₋₆alkylene-(X¹—C₁₋₆ alkylene)₁₋₂₄-X¹ C₁₋₆alkyl, —C₂alkylene-(X¹—C₁₋₆alkylene)₁₋₂₄-X¹—C₁₋₆alkyl, —C₃alkylene-(X—C₁₋₆ alkylene)₁₋₂₄-X¹—C₁₋₆alkyl, —C₄alkylene-(X¹—C₁₋₆alkylene)₁₋₂₄-X¹—C₁₋₆alkyl, —C₅alkylene-(X¹—C₁₋₆ alkylene)₁₋₂₄-X¹—C₁₋₆alkyl, and —C₆alkylene-(X¹—C₁₋₆alkylene)₁₋₂₄-X¹—C₁₋₆alkyl.

14. The compound or salt of claim 13, wherein R²³ is selected from —C₂alkylene-(X¹—C₁₋₆ alkylene)₁₋₂₄-X¹—C₁₋₆alkyl.

15. The compound or salt of claim 13, wherein R²³ is selected from —C₂alkylene-(X¹—C₁₋₆ alkylene)₂-X¹—C₁₋₆alkyl, —C₂alkylene-(X¹—C₁₋₆alkylene)₅-X¹—C₁₋₆alkyl, —C₂alkylene-(X¹—C₁₋₆alkylene)₁₁-X¹—C₁₋₆alkyl, and —C₂alkylene-(X¹—C₁₋₆alkylene)₁₆-X¹—C₁₋₆alkyl.

16. The compound or salt of any one of claims 12 to 15, wherein each X¹ is O.

17. The compound or salt of claim 11, wherein R²³ is selected from a 3- to 12-membered heterocycle optionally substituted with one or more substituents independently selected at each occurrence from R²⁴.

18. The compound or salt of claim 17, wherein R²³ is selected from a 5-membered heterocycle optionally substituted with one or more substituents independently selected at each occurrence from R²⁴.

19. The compound or salt of claim 18, wherein R²³ is selected from: 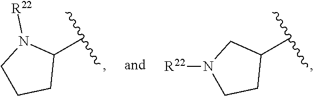
wherein R²³ is optionally substituted with one or more substituents independently selected at each occurrence from R²⁴.

20. The compound or salt of claim 17, wherein R²³ is selected from a 6-membered heterocycle optionally substituted with one or more substituents independently selected at each occurrence from R²⁴.

21. The compound or salt of claim 20, wherein R²³ is selected from: 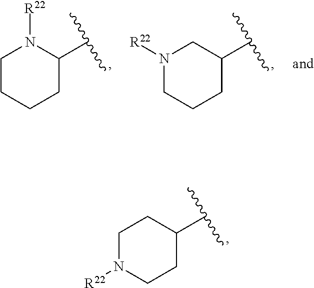
wherein R²³ is optionally substituted with one or more substituents independently selected at each occurrence from R²⁴.

22. The compound or salt of claim 20, wherein R²³ is selected from: 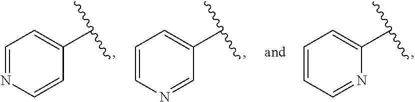
R²³ is optionally substituted with one or more substituents independently selected at each occurrence from R²⁴.

23. The compound or salt of claim 2, wherein R¹ and R¹⁰ are independently selected from hydrogen and —C(O)(C(R²⁰)₂)₁₋₆OC(O)R²⁰.

24. The compound or salt of claim 2, wherein R¹ and R¹⁰ are independently selected from hydrogen and —C(O)(C(R²⁰)₂)₁₋₆C(O)OR²¹.

25. The compound or salt of claim 24, wherein R¹ and R¹⁰ are independently selected from hydrogen, —C(O)C(R²⁰)₂C(O)OR²¹, —C(O)(C(R²⁰)₂)₂C(O)OR²¹, —C(O)(C(R²⁰)₂)₃C(O)OR²¹, —C(O)(C(R²⁰)₂)₄C(O)OR²¹, —C(O)(C(R²⁰)₂)₅C(O)OR²¹, and —C(O)(C(R²⁰)₂)₆C(O)OR²¹.

26. The compound or salt of claim 25, wherein R¹ and R¹⁰ are independently selected from hydrogen, —C(O)(C(R²⁰)₂)₂C(O)OR²¹ and —C(O)(C(R²⁰)₂)₃C(O)OR²¹.

27. The compound or salt of claim 26, wherein R¹ and R¹⁰ are independently selected from hydrogen,

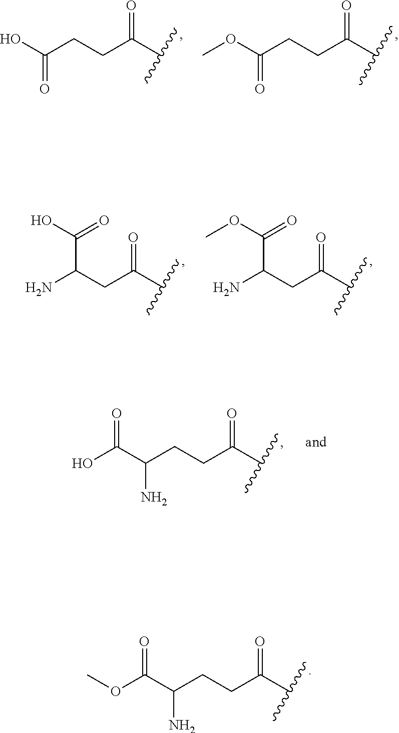

28. The compound or salt of claim 2, wherein R¹ and R¹⁰ are independently selected from hydrogen and —C(O)OC(R²⁰)₃.

29. The compound or salt of claim 28, wherein R¹ is —C(O)OC(R²⁰)₃ and R¹⁰ is hydrogen.

30. The compound or salt of claim 29, wherein R¹ is

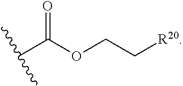

31. The compound or salt of claim 28, 29 or 30, wherein R²⁰ is independently selected at each occurrence from:
hydrogen, 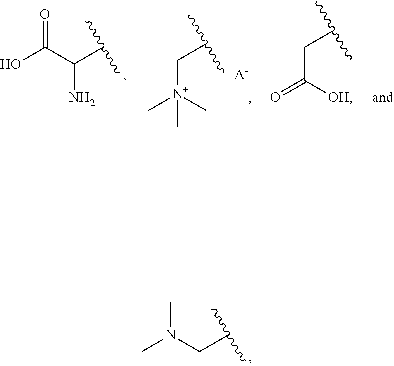
wherein A⁻ is an anion.

32. The compound or salt of claim 31, wherein R¹ is

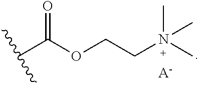

33. The compound or salt of claim 2, wherein R¹ and R¹⁰ are independently selected from hydrogen and —P(═O)(X²R²¹)₂.

34. The compound or salt of claim 33, wherein each X² is O.

35. The compound or salt of claim 33, wherein R²¹ is independently selected at each occurrence from hydrogen and C₁₋₆ alkyl.

36. The compound or salt of claim 33, wherein R¹ and R¹⁰ are independently selected from hydrogen,

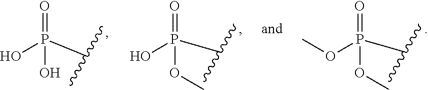

37. The compound or salt of claim 2, wherein R¹ and R¹⁰ are independently selected from hydrogen and —CH₂OP(═O)(X²R²¹)₂.

38. The compound or salt of claim 37, wherein each X² is O.

39. The compound or salt of claim 37, wherein R²¹ is independently selected from hydrogen and C₁₋₆ alkyl.

40. The compound or salt of claim 37, wherein R¹ and R¹⁰ are independently selected from hydrogen,

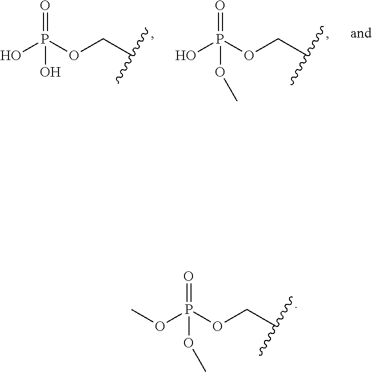

41. The compound or salt according to any one of claims 1-40, wherein R², R³, R⁴, and R⁵ are independently selected at each occurrence from halogen, —OR¹⁵, —N(R¹⁵)₂, —C(O)R¹⁵, —C(O)OR¹⁵, —C(O)N(R¹⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN.

42. The compound or salt according to claim 41, wherein R², R³, R⁴, and R⁵ are independently selected at each occurrence from halogen, —OR¹⁵, —N(R¹⁵)₂, —NO₂, —CN, and C₁-C₆ alkyl optionally substituted with one or more substituents independently selected from halogen, —OR¹⁵, —N(R¹⁵)₂, —NO₂, and —CN.

43. The compound or salt according to claim 42, wherein R², R³, R⁴, and R⁵ are independently selected at each occurrence from halogen, —OR¹⁵, and C₁-C₆ alkyl optionally substituted with one or more substituents independently selected from halogen, —OR¹⁵, and —CN.

44. The compound or salt according to any one of claims 1-43, wherein R⁷ and R⁸ are independently selected at each occurrence from hydrogen, halogen, —OR¹⁵, —N(R¹⁵)₂, —C(O)R¹⁵, —C(O)OR¹⁵, —C(O)N(R⁵)₂, —NO₂, —CN, and C₁-C₆ alkyl optionally substituted with one or more substituents independently selected from halogen, —OR¹⁵, —SR¹⁵, —N(R¹⁵)₂, —NO₂, and —CN.

45. The compound or salt according to claim 44, wherein R⁷ and R⁸ are independently selected at each occurrence from hydrogen, halogen, and C₁-C₆ alkyl optionally substituted with one or more substituents independently selected from halogen, —OR¹⁵, —N(R¹⁵)₂, —NO₂, and —CN.

46. The compound or salt according to claim 45, wherein R⁷ and R⁸ are hydrogen.

47. The compound or salt according to any one of claims 1-46, wherein n is selected from 0 and 1.

48. The compound or salt according to any one of claims 1-47, wherein s is selected from 0 and 1.

49. The compound or salt according to any one of claims 1-48, wherein m is selected from 0 and 1.

50. The compound or salt according to any one of claims 1-49, wherein t is selected from 0 and 1.

51. The compound or salt according to any one of claims 1-50, wherein R⁶, R⁹, R¹¹, R¹², and R¹³ are independently selected from C₁-C₃ alkyl optionally substituted with one or more substituents independently selected from halogen, —OR¹⁵, —SR¹⁵, —N(R¹⁵)₂, —NO₂, and —CN.

52. The compound or salt according to claim 51, wherein R⁶, R⁹, R¹¹, R¹², and R¹³ are independently selected from methyl optionally substituted with one or more substituents independently selected from halogen, —OR¹⁵, —N(R¹⁵)₂, —NO₂, and —CN.

53. The compound or salt according to claim 52, wherein R⁶, R⁹, R¹¹, R¹², and R¹³ are each methyl.

54. The compound or salt according to claim 1, selected from: 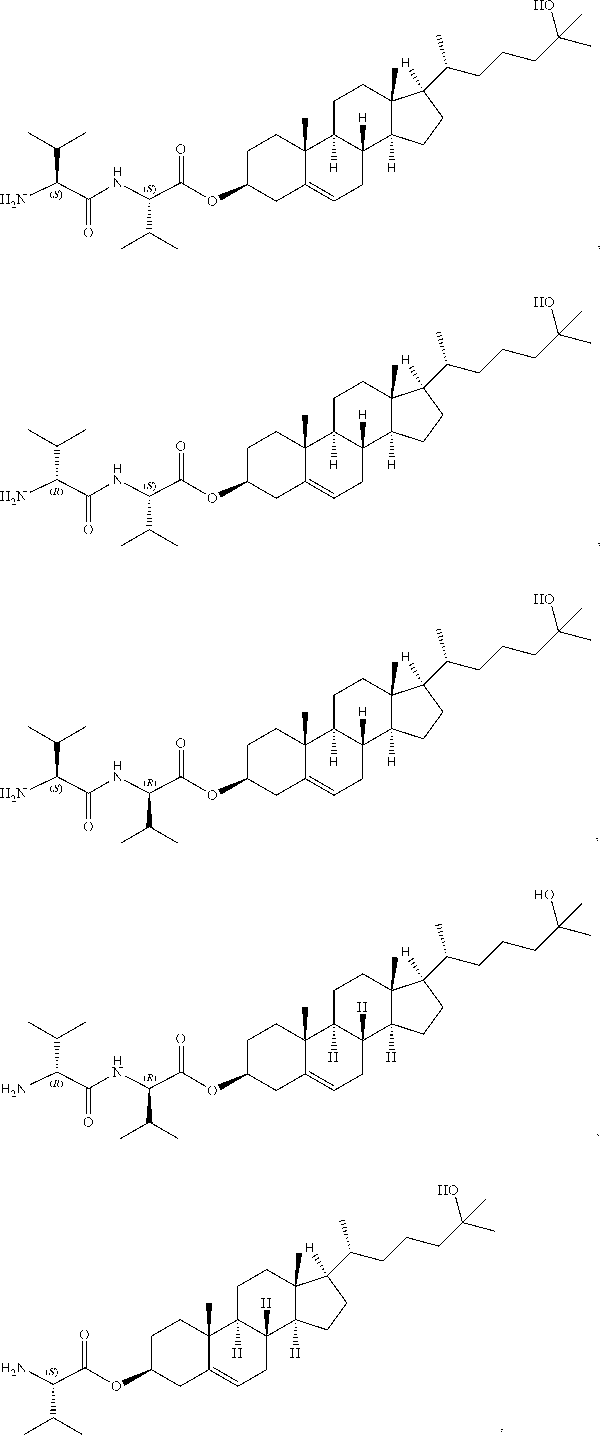 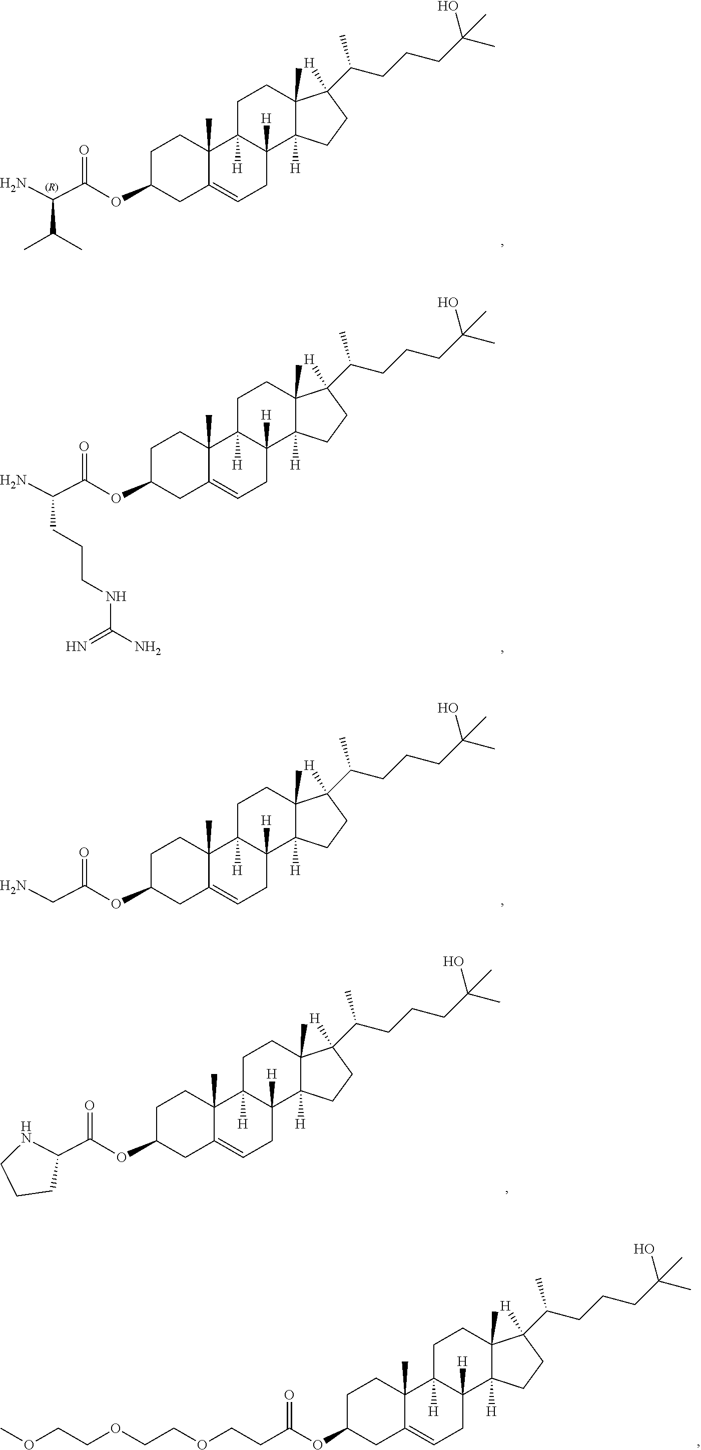 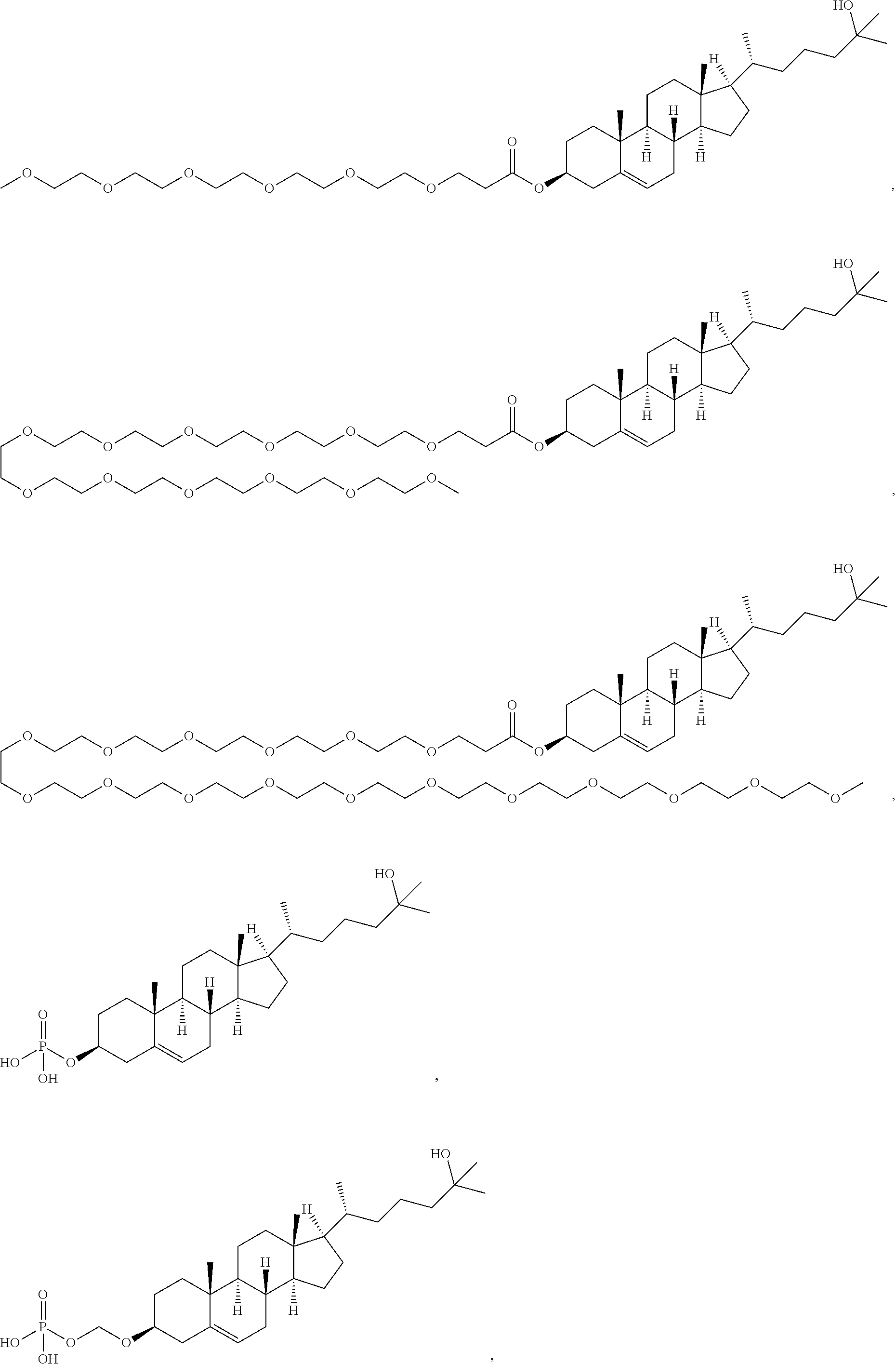 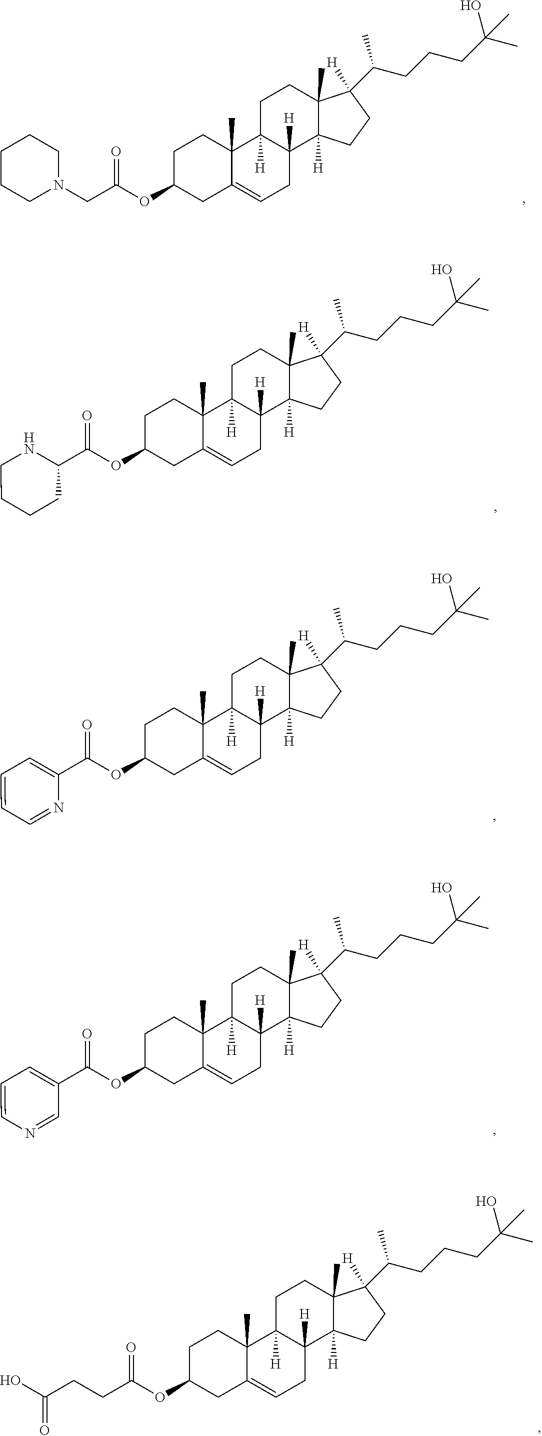 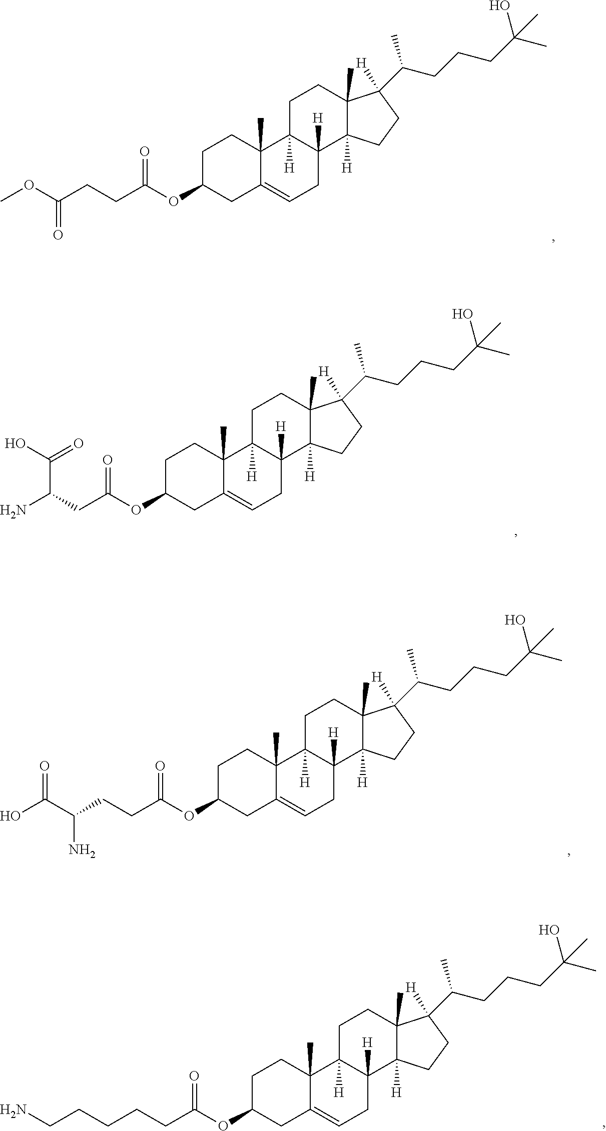 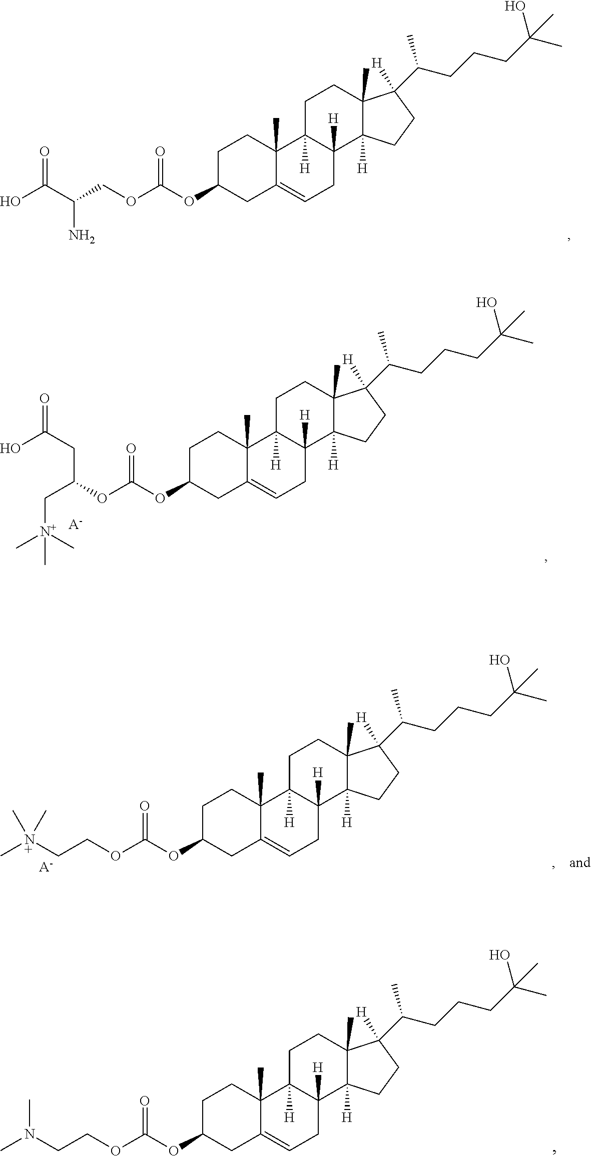
wherein A⁻ is an anion, or a salt of any one thereof.

55. A compound or salt of claim 1, wherein:
R¹ is selected from —C(O)(CR²⁰₂)₁₋₆NR²¹₂, —C(O)R²³, —C(O)(C(R²⁰)₂)₁₋₆OC(O)R²⁰, —C(O)(C(R²⁰)₂)₁₋₆C(O)OR²¹, —C(O)OC(R²⁰)₃, —P(═O)(X²R²¹)₂, and —CH₂OP(═O)(X²R²¹)₂;
R¹⁰ is hydrogen;
R², R³, R⁴, and R⁵ are independently selected at each occurrence from halogen, —OR, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;
R⁷ and R⁸ are independently selected at each occurrence from hydrogen, halogen, —OR, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;
n, s, m, and t are each 0, 1, or 2;
R⁶, R⁹, R, R¹², and R¹³ are independently selected at each occurrence from C₁-C₆ alkyl optionally substituted with one or more substituents independently selected from halogen, —OR¹⁵, —SR¹⁵, —N(R¹⁵)₂, —NO₂, and —CN;
R¹⁵ is independently selected at each occurrence from hydrogen, and C₁-C₃ alkyl optionally substituted with one or more substituents independently selected from halogen, —CN, —NO₂, —NH₂, —OH, and —OCH₃;
R²⁰ is independently selected at each occurrence from hydrogen, halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;
R²¹ is selected from hydrogen, —C(O)R²², —C(O)OR²² and —C(O)N(R²²)—; C₁₋₆ alkyl, C₂₋₆ alkenyl, and C₂₋₆ alkynyl, each of which is independently optionally substituted at each occurrence with one or more substituents selected from halogen, —NO₂, —CN, —OR²², —SR²², —S—S—R²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —S—C(O)R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is optionally substituted with one or more substituents independently selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl;
or two R²¹ groups are taken together with the atoms to which they are attached form a heterocycle, optionally substituted with one or more R²⁴;
R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃;
R²³ is selected from:
—C₁₋₆alkylene-(X¹—C₁₋₆alkylene)₁₋₂₄-X¹—C₁₋₆alkyl, wherein X¹ is independently selected at each occurrence from O, S, and NH and wherein R²³ is optionally substituted on a carbon or nitrogen atom with one or more substituents independently selected from R²⁴; and
a 3- to 12-membered heterocycle optionally substituted with one or more substituents independently selected from R²⁴;
R²⁴ is independently selected at each occurrence from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle;
and C₃₋₁₂ carbocycle and 3- to 12-membered heterocycle, wherein each C₃₋₁₂ carbocycle and 3- to 12-membered heterocycle is independently optionally substituted at each occurrence with one or more substituents selected from halogen, —NO₂, —CN, —OR²², —SR²², —N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₁₋₆ alkyl, C₁₋₆ haloalkyl, C₂₋₆ alkenyl, and C₂₋₆ alkynyl; and
X² is independently selected at each occurrence from O and NH.

56. The compound or salt of claim 55, wherein:
R¹ is selected from —C(O)(C(R²⁰)₂)₁₋₆C(O)OR²¹ and —C(O)OC(R²⁰)₃;
R¹⁰ is hydrogen;
R², R³, R⁴, and R⁵ are independently selected at each occurrence from halogen, —OR¹⁵, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;
R⁷ and R⁸ are each hydrogen;
n, s, m, and t are each 0, 1, or 2;
R⁶, R⁹, R¹¹, R¹², and R¹³ are independently selected at each occurrence from C₁-C₃ alkyl optionally substituted with one or more substituents independently selected from halogen, —OR¹⁵, —SR¹⁵, —N(R¹⁵)₂, —NO₂, and —CN;
R¹⁵ is independently selected at each occurrence from hydrogen, and C₁-C₃ alkyl optionally substituted with one or more substituents independently selected from halogen, —CN, —NO₂, —NH₂, —OH, and —OCH₃;
R²⁰ is independently selected at each occurrence from hydrogen, halogen, —NO₂, —CN, —OR²², —SR²², —N(R²²)₂, —P(O)(OR²²)₂, —OP(O)(OR²²)₂; C₁₋₆ alkyl optionally substituted with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle;
R²¹ is selected from hydrogen; and C₁₋₆ alkyl optionally substituted at each occurrence with one or more substituents selected from halogen, —NO₂, —CN, —OR²², —SR²², —N(R²²)₂, and —N(R²²)₃⁺; and
R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃.

57. The compound or salt of claim 56, wherein:
R¹ is —C(O)(C(R²⁰)₂)₁₋₄C(O)OR²¹;
R¹⁰ is hydrogen;
R², R³, R⁴, and R⁵ are independently selected at each occurrence from halogen, —OR¹⁵, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;
R⁷ and R⁸ are each hydrogen;
n, s, m, and t are each 0, 1, or 2, such as each of n, s, m, and t are 0;
R⁶, R⁹, R¹¹, R¹², and R¹³ are independently selected at each occurrence from C₁-C₃ alkyl optionally substituted with one or more substituents independently selected from halogen, —OR¹⁵, —SR¹⁵, —N(R¹⁵)₂, —NO₂, and —CN, such as each of R⁶, R⁹, R¹¹, R¹², and R¹³ are CH₃;
R¹⁵ is independently selected at each occurrence from hydrogen, and C₁-C₃ alkyl optionally substituted with one or more substituents independently selected from halogen, —CN, —NO₂, —NH₂, —OH, and —OCH₃;
R²⁰ is independently selected at each occurrence from hydrogen, halogen, —NO₂, —CN, —OR²², —SR²², —N(R²²)₂, —P(O)(OR²²)₂, —OP(O)(OR²²)₂; C₁₋₆ alkyl optionally substituted with one or more substituents selected from halogen, —NO₂, —CN, —OR²², —SR²², —N(R²²)₂, —N(R²²)₃⁺, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle, such as each R²⁰ is selected from hydrogen and —N(R²²)₂;
R²¹ is selected from hydrogen; and C₁₋₆ alkyl optionally substituted at each occurrence with one or more substituents selected from halogen, —NO₂, —CN, —OR²², —SR²², —N(R²²)₂, and —N(R²²)₃⁺ such as R²¹ is methyl or hydrogen; and
R²² is independently selected at each occurrence from hydrogen; and C₁₋₆ alkyl, C₃₋₁₂ carbocycle and 3- to 12-membered heterocycle, each of which may be optionally substituted at each occurrence by halogen, —CN, —NO₂, —OH, —NH₂, and —OCH₃.

58. The compound of claim 57, represented by the structure: 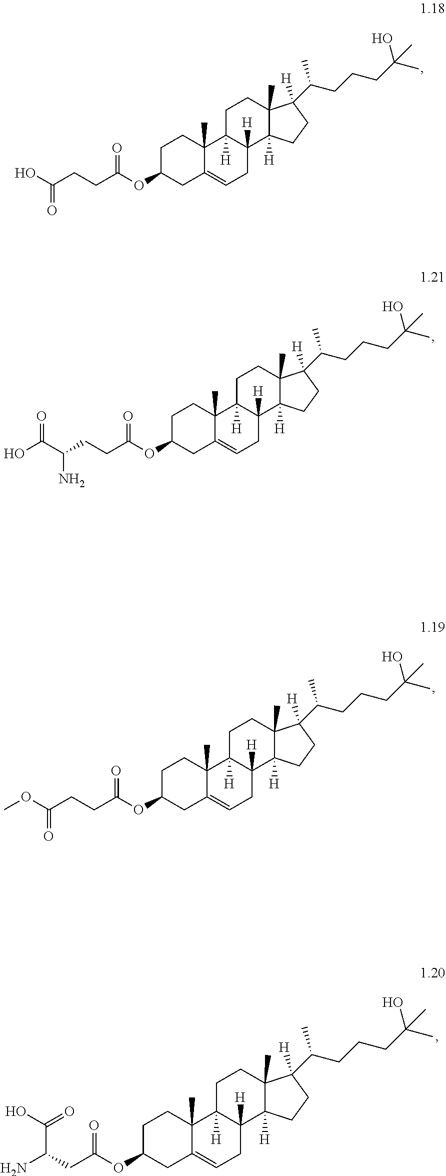
a salt of any one thereof.

59. The compound of claim 58, represented by the structure: 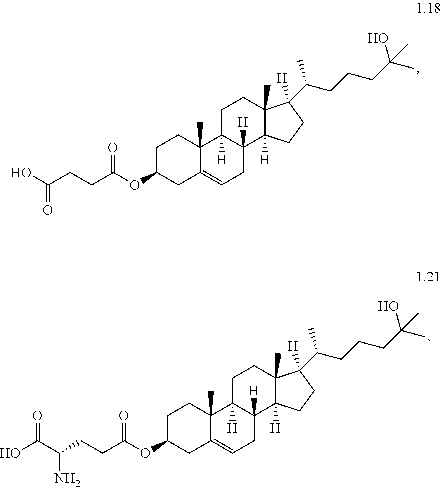
or a salt of any one thereof.

60. The compound or salt of claim 56, wherein:
R¹ is selected from —C(O)OC(R²⁰)₃;
R¹⁰ is hydrogen;
R², R³, R⁴, and R⁵ are independently selected at each occurrence from halogen, —OR¹⁵, —SR¹⁵, —N(R¹⁵)₂, —NO₂, —CN, and C₁-C₃ alkyl optionally substituted with one or more substituents independently selected from halogen, —OR¹⁵, —SR¹⁵, —N(R¹⁵)₂, —NO₂, and —CN;
R⁷ and R⁸ are each hydrogen;
n, s, m, and t are each 0, 1, or 2, such as each of n, s, m, and t are 0;
R⁶, R⁹, R¹¹, R¹², and R¹³ are independently selected at each occurrence from C₁-C₃ alkyl optionally substituted with one or more substituents independently selected from halogen, —OR¹⁵, —SR¹⁵, —N(R¹⁵)₂, —NO₂, and —CN, such as each of R⁶, R⁹, R¹¹, R¹², and R¹³ are methyl;
R¹⁵ is independently selected at each occurrence from hydrogen, and C₁-C₃ alkyl optionally substituted with one or more substituents independently selected from halogen, —CN, —NO₂, —NH₂, —OH, and —OCH₃;
R²⁰ is independently selected at each occurrence from hydrogen, halogen, —NO₂, —CN, —OR²², —SR²², —N(R²²)₂, —P(O)(OR²²)₂, —OP(O)(OR²²)₂; C₁₋₆ alkyl optionally substituted with one or more substituents selected from halogen, —NO₂, —CN, —OR²², —SR²², —N(R²²)₂, —N(R²²)₃, —NR²²C(═N)N(R²²)₂, —S(═O)R²², —S(═O)₂R²², —S(═O)₂N(R²²)₂, —NR²²S(═O)₂R²², —C(O)R²², —C(O)OR²², —OC(O)R²², —OC(O)OR²², —OC(O)N(R²²)₂, —NR²²C(O)R²², —C(O)N(R²²)₂, ═O, ═S, ═N(R²²), —P(O)(OR²²)₂, —OP(O)(OR²²)₂, C₃₋₁₂ carbocycle and 3- to 12-membered heterocycle, such as each R²⁰ is independently hydrogen or C₁₋₆ alkyl optionally substituted with one or more substituents selected from halogen, —NO₂, —CN, —OR²², —SR²², —N(R²²)₂, —N(R²²)₃⁺; and
R²² is independently selected at each occurrence from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted at each occurrence by halogen, —CN, —NO₂, —OH, —NH₂, and —OCH₃; such as each R²² is methyl.

61. The compound of claim 60, represented by the structure: 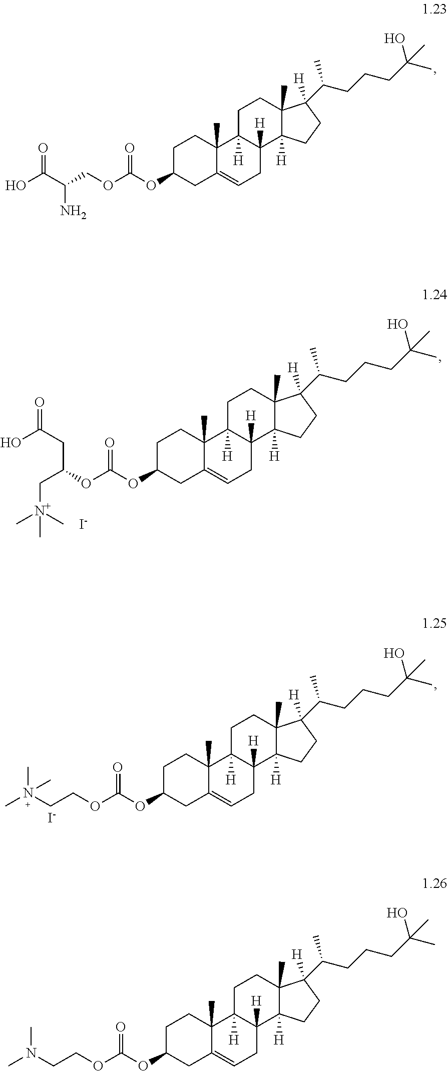
or another salt one thereof.

62. The compound of claim 61, represented by the structure: 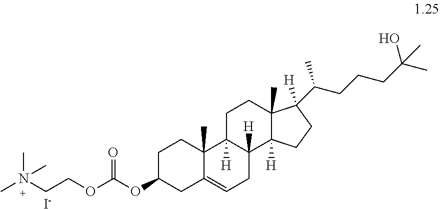
or another salt thereof.

63. The compound or salt of any one of claims 1 to 62, wherein the compound or salt has a chemical stability of 400 min or greater, 450 minutes or greater, or 500 minutes or greater as evaluated using the chemical stability procedure in EXAMPLE 21 in the Examples section herein.

64. The compound or salt of any one of claims 1 to 62, wherein the compound or salt has a plasma stability of 400 min or greater, 450 minutes or greater, or 500 minutes or greater as evaluated using the chemical stability procedure in EXAMPLE 21 in the Examples section herein.

65. A compound or salt according to any one of claims 1-62 which demonstrates 10% or more increased aqueous solubility relative to 25-hydroxycholesterol, wherein the compound or salt according to any one of claims 1-51 and 25-hydroxycholesterol are subjected to the solubility assay:
a. weigh an excess of sample into an Eppendorf tube;
b. add 0.5 mL of water for injection to the Eppendorf tube;
c. incubate the Eppendorf tube at 37° C. for 6 h shaking at 200 rpm;
d. centrifuge the Eppendorf tube for 10 min at 13,500 rpm;
e. pipette 250 μL of liquid and filter through 0.45 m disposable syringe filter;
f collect the filtrate and dilute further with water for injection;
g. measure the absorbance of the filtrate by UV spectroscopy;
h. calculate solubility in mg/mL; and
compare the solubility in step h. of the compound or salt of Formula (I) with the solubility in step h. of 25-hydroxycholesterol.

66. A compound or salt according to any one of claims 1-62 which demonstrates 10% or more increased cellular uptake relative to 25-hydroxycholesterol, wherein the compound or salt according to any one of claims 1-62 and 25-hydroxycholesterol are each subjected to the cellular uptake assay:
a. incubate a sample with cultured primary rabbit corneal cells in DPBS for 10 minutes;
b. wash the cells three times with ice-cold HEPES buffer;
c. lyse the cells overnight with 1 mL 0.05% (w/v) Triton X-100 in 1 N NaOH at room temperature;
d. transfer 500 μL aliquots from each well to scintillation vials containing 5 mL scintillation cocktail;
e. subject the sample to liquid scintillation spectrophotometry using a scintillation counter;
f normalize the rate of uptake to the protein content of each well; and
g. measure the amount of protein in the cell lysate using bovine serum albumin as the standard; and
compare the measured amount of protein in the cell lysate in step g. of the compound or salt according to any one of claims 1-62 with the measured amount of protein in the cell lysate in step g. of 25-hydroxycholesterol.

67. A compound or salt according to any one of claims 1-62, which demonstrates 10% or more increased corneal diffusion relative to 25-hydroxycholesterol.

68. A pharmaceutical composition comprising a compound or salt according to any one of claims 1-62 and a pharmaceutically acceptable carrier.

69. A pharmaceutical composition according to claim 68, wherein the pharmaceutical composition is an eye drop.

70. A method of treating a near-vision disorder in a subject in need thereof, comprising administering to the subject a pharmaceutical composition according to claim 68 or 69.

71. The method according to claim 70, wherein the near-vision disorder is selected from cataract and presbyopia.

72. The method according to claim 71, wherein cataract is selected from nuclear cataract, cortical cataract, posterior capsular cataract, congenital cataract, early-onset hereditary cataract, metabolic cataract, secondary cataract, blunt traumatic cataract, penetrating traumatic cataract, post-vitrectomy cataract, and radiation-induced cataract.

73. The method according to claim 71, wherein presbyopia is selected from incipient, presbyopia, functional presbyopia, absolute presbyopia, premature presbyopia and nocturnal presbyopia.

74. The method according to any one of claims 70 to 73, wherein administration of the pharmaceutical composition comprises topical administration of the pharmaceutical composition.

75. The method of claim 74, wherein topical administration is administration to the surface of the eye of said subject.

76. The method of any one of claims 70 to 73, wherein the method further comprises administering an antioxidant.

77. The method of claim 76, wherein the antioxidant comprises alpha-lipoic acid or a prodrug thereof.
